(19) United States
(12) Patent Application Publication
(10) Pub. No.: US 2006/0073574 A1
(43) Pub. Date: Apr. 6, 2006

(54) Engineered biosynthesis of novel polyenes

(76) Inventor: John Patrick Caffrey, Greystones (IE)

Correspondence Address: Brian S. Steinberger, 101 Brevard Avenue, Cocoa, FL, 32922, US

(21) Appl. No.: 10/478,943

(22) PCT Filed: May 27, 2002
(86) PCT No.: PCT/IE02/00071

§ 371 Date: Nov. 26, 2003

(30) Foreign Application Priority Data
May 31, 2001 (US) S2001/0527

(51) Int. Cl. C07H 17/08; C07H 21/04; C12P 21/06; C12P 19/62; C12N 9/02; C12N 9/10; C12N 15/74

(52) U.S. Cl. 435/76; 435/69.1; 435/252.3; 435/193; 435/189; 435/471; 536/23.2; 536/6.5

(57) ABSTRACT

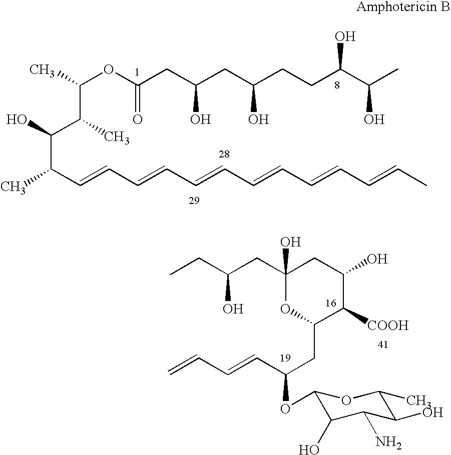

The complete DNA sequence of the gene cluster encoding the polypeptides responsible for the biosynthesis of the polyene antibiotic amphotericin of S. nodosus is provided. Engineered alteration of the amphC) gene so as to eliminate production of amphotericin A in favour of the more active form amphotericin B. as well as the production of amphotericin analogues having altered characteristics, is enabled by manipulation of the sequences of the cluster.

The present invention relates to the biosynthetic gene cluster that governs the production of the polyene antibiotic amphotericin in Streptomyces nodosus, to the nucleic acid sequence thereof and to the use of all or part of the cloned DNA in the production of bioactive molecules from streptomycetes.

Polyketides are natural products formed by stepwise condensation of small carboxylic acids. The group is large and structurally diverse and includes hundreds of bioactive compounds like antibacterial and antifungal antibiotics, anticancer drugs and unosuppressants. Polyketides are produced by a wide range of plants and micro-organisms but the most prolific producers are the Streptomyces genus of soil bacteria.

Polyketides are synthesised by a process that resembles the biosynthesis of saturated fatty acids (Hopwood, D. A. and Sherman, D. H. Annu. Rev. Genet. (1990) 24: 37-66). The carbon chains are assembled in a series of extension cycles in which two-carbon units are added to an acyl chain. In each cycle, an acyl unit is loaded onto the active site cysteine thiol of a ketosynthase (KS) domain. An acyltransferase (AT) transfers a malonyl, methylmalonyl or ethylmalonyl extender acyl unit from CoA onto the phosphopantetheine thiol of an acyl carrier protein (ACP) domain, Decarboxylative condensation then gives a β-ketoacyl chain thioester-linked to the ACP. Up to three processing reactions may then occur. A ketoreductase (KR) domain reduces the β-ketone group to give a α-hydroxyacyl chain. A dehydratase DH domain catalyses formation of an α-β unsaturated acyl chain. The resulting enoyl group may then be reduced by an enoyl reductase (ER) domain to give a saturated acyl chain. In fatty acid biosynthesis, each cycle of synthesis is the same. The starter unit is usually acetate, malonate is invariably used as extender unit and the α-carbonyl group is almost always completely processed to a methylene group. The end product is typically a saturated fatty acyl chain. In polyketide biosynthesis, differing extension cycles generate a greater diversity of structures. A wider range of starter and extender units is used and β-ketone processing steps may be omitted so that ketone, hydroxyl and enoyl groups appear in the chains. The incorporation of methylmalonyl or ethylmalonyl extender units introduce methyl or ethyl branches into the chain. Carbon atoms bearing these side chains are chiral, as are carbon atoms with hydroxyl groups. The stereochemistry at these centres is determined during each cycle of chain extension (Marsden et al. Science (1994) 263: 378-380; Kao et al. J. Am. Chem. Soc. (1998): 120:2478-2479; Bohm, I. et al. Chem. Biol. 5:407-412)

Polyketides fall into two main structural groups: aromatic and complex. Aromatic polyketides include the antibiotic oxytetracycline, the anticancer compounds tetracenomycin and daunorubicin, and actinorhodin, a blue pigment made by Streptomyces coelicolor.

Aromatic polyketides are synthesised from acetate (malonyl) units and the β-ketone groups formed in each cycle are largely unprocessed. The initial product is a highly reactive poly β-carbonyl chain. The alternating methylene and ketone groups promote intramolecular aldol condensations that eventually lead to the formation of aromatic rings. Biosynthetic gene clusters for several aromatic polyketides have now been sequenced (Bibb, M. J. et al. EMBO J. (1989) 8:2727-2736; Sherman, D. H. et al EMBO J. (1989) 8:2717-2725; Fernandez-Moreno, M. A. et al. J. Biol. Chem. (1992) 267:19278-19290). Aromatic or type II polyketide synthases PKSs characteristically consist of three discrete proteins, KS-α, KS-β and an ACP. The KS-α and ACP co-operate in carbon-carbon bond formation. The KS-β resembles a normal ketosynthase except that the active site cysteine is replaced by glutamine. The KS-β enzyme functions as a decarboxylase that generates acetyl primer units from malonyl ACP (Bisang et al., Nature (1999) 401(6752): 502-505). The gene clusters do not contain genes for malonyl transferases. Aromatic PKSs are thought to use the malonyl transferase that normally functions in fatty acid biosynthesis (Revill W. P. et al. J. Bacteriol. (1995) 177: 3946-3952). Some purified type II PKS ACPs have been shown to be capable of self-malonylation in vitro in the presence of high concentrations of malonyl-CoA (Hitchman et al., Chemistry and Biology (1998) δ: 35-47).

Some aromatic PKS gene clusters may also contain a gene for a KR that specifically reduces a single ketone group, usually at C-9 within the growing chain. There are also genes for cyclases that direct the pattern of ring formation and aromatases that catalyse dehydration reactions that aromatise the rings (Hutchinson, C. R. and Fujii, I. Ann. Rev.

Microbiol. (1995) 49:201-238). Additional genes are required for further modifications of the product, for export and resistance.

International Patent Application Number WO 95/08548 describes pRM5, a vector devised for heterologous expression of natural and hybrid aromatic polyketide biosynthetic gene clusters. This is based on the low copy number vector SCP2* plasmid from Streptomyces coelicolor (Bibb, M. J. and Hopwood, D. A. J. Gen. Microbiol. (1981) 126:427-442). Plasmid pRM5 contains the divergent act I/act m promoter region of the actinorhodin cluster (Fernandez-Moreno, M. A. et al. J. Biol. Chem. (1992) 267:19278-19290) and the act II ORF4 transcriptional activator that activates transcription from this promoter during the transition from exponential growth to stationary phase (Hallam, S. E. et al. Gene (1988) 74:305-320).

International Patent Application Number WO 95/08548 also describes S. coelicolor CH999, a host strain developed for expression of heterologous PKS genes cloned in pRM5. The chromosomal actinorhodin genes have been deleted from this strain. Expression of various combinations of aromatic PKS, cyclase and aromatase genes in this host-vector system has led to synthesis of novel compounds (Hopwood, D. A. Chem. Rev. (1997): 97: 2465-2478).

Complex polyketides are represented by the macrolides erythromycin, oleandomycin, avermectin and rapamycin. These polyketides are assembled by Type I or modular polyketide synthases. These enzyme systems contain a synthase unit or module for each cycle of chain extension (Cortés, J. et al. Nature (1990) 348:176-178; Donadio, S. et al. Science (1991) 252:675-679; Swan, D. G. et al. Mol. Gen. Genet. (1994) 242:358-362; MacNeil, D. J. et al. Gene (1992) 115:119-125; Schwecke, T. et al. Proc. Natl. Acad. Sci. USA (1995) 92:7839-7843). Each extension module contains AT, KS and ACP domains, the minimum requirements for catalysis of chain growth. The AT domains may be specific for malonyl, methylmalonyl or ethylmalonyl groups and select the extender unit appropriate for the cycle. A module may also contain reduction domains. These determine the extent of β-ketone group processing. A KR domain alone specifies a hydroxyl group, KR plus DH domains specify an enoyl group and a full complement of KR, DH and ER domains specifies a methylene group. These domains are housed within multienzyme polypetides and appear in the order KS-AT-DH-ER-KR-ACP in a complete module. The reduction domains may be absent or present in an inactive form. A type I PKS protein may contain one or more modules. On completion of a cycle the extended chain is passed from the ACP to the KS of the next module. The total number of modules in the PKS determines the chain length. The completed chains are usually cyclised and released by thioesterase domains.

Polyketide macrolactone rings frequently undergo further modifications which include hydroxylation by cytochrome P450 enzymes, glycosylation with neutral or amino sugars, and methylation by O- or C-methyl transferases. These post-polyketide modifications are usually catalysed by discrete enzymes but C-methyl transferases may be housed as an additional domain within an extension module. This has been seen in the epothilone PKS of the myxobacterium Sorangium cellulosum. This PKS contains a C-methyltransferase domain embedded within extension module 8 (Tang et al. Science (2000) 287: 640-642).

Genetic manipulation of specific modules can lead to biosynthesis of novel polyketides with structural alterations at chemically defined positions. Engineering of reduction domains can in principle allow interchange of ketone, hydroxyl, enoyl and methylene groups. The KR5 domain of the erythromycin PKS of Saccharopolyspora erythraea was specifically inactivated by targeted gene disruption. The mutant strain produced the erythromycin analogues 5,6-dideoxy-3-α-mycarosyl-5-oxoerythronolide B, 5,6-dideoxy-5-oxoerythronolide B and 5,6-dideoxy,6-p-epoxy-5-oxoerythronolide B (Donadio, S. et al. Science (1991) 252:675-679). Inactivation of the NADPH-binding site of the ER domain of module 4 gave a mutant enzyme that synthesised Δ 6, 7 anhydroerythromycin C (Donadio, S. et al. Proc. Natl. Acad. Sci. USA (1993) 90:7119-7123). Further examples of manipulation of β-ketone group processing have been described (Khosla, C. et al., Annu.

Rev. Biochem. (1999) 68: 219-253). Exchanging AT domains can result in synthesis of analogues lacking methyl side chains (Khosla, C. et al., Annu. Rev. Biochem. (1999) 68: 219-253). Replacement of the AT4 of the erythromycin PKS with an ethylmalonate-specific AT resulted in biosynthesis of an ethylated erythromycin analogue (Stassi et al., Proc. Natl. Acad. Sci. USA (1998) 95: 7305-7309). Production of this novel compound was dependent on overproduction of crotonyl CoA reductase in the mutant S. erythraea strain. This enzyme apparently increases in the intracellular level of butyryl CoA, the precursor of ethylmalonyl CoA.

The chain lengths of complex polyketides can be reduced by genetically fusing chain-terminating thioesterase domains to internal extension modules (Cortes J. et al., Science (1995) 268: 1487-1489; Kao, C. M., et al. J. Am. Chem. Soc. (1995) 117:9105-9106).

Type I PKSs also incorporate loading modules. This is a group of domains that transfers the starter unit onto the KS of the first extension module. Novel compounds can also be generated by exchanging loading modules to alter the primer specificity of a PKS. WO98/01560 describes replacement of the loading module of the erythromycin PKS with the broad-specificity loading module from the avermectin-producing PKS (see also Marsden, A. F. A. et al. Science (1998) 279:199-202). Certain novel polyketides can be prepared using the hybrid PKS gene assembly, as described for example in WO98/01560, which further describes the construction of a hybrid PKS gene assembly by grafting the loading module from the rapamycin PKS onto the first module of the erythromycin PKS. The rapamycin loading module is unusual in that it consists of a CoA ligase domain, an enoylreductase (“ER”) domain and an ACP. Suitable organic acids including the natural starter unit 3,4-dihydroxycyclohexane carboxylic acid may be activated in situ on the PKS loading domain and, with or without reduction by the ER domain, transferred to the ACP for intramolecular loading of the KS of extension module 1 (Schwecke, T. et al. Proc. Natl. Acad. Sci. USA (1995) 92:7839-7843). Published International Patent Applications numbers WO 98/51695, WO 98/49315 and WO 93/13663 describe additional types of genetic manipulation of the erythromycin PKS genes that are capable of producing altered polyketides. However many such attempts are reported to have been unproductive (Hutchinson, C. R. and Fujii, I. Annu. Rev. Microbiol. (1995) 49:201-238, at p. 231).

The DNA sequences have also been disclosed for several Type I PKS gene clusters that govern the production of 16-membered macrolide polyketides, including the tylosin PKS from Streptomyces fradiae (EP-A-0 791 655 A2), the niddamycin PKS from Streptomyces caelestis (Kavakas, S. J. et al. J. Bacteriol. (1997) 179:7515-7522) and the spiramycin PKS from Streptomyces ambofaciens (EP-A-0791 655 A2). DNA sequences have also been disclosed for Type I PKS gene clusters that govern the production of further complex polyketides, for example rifamycin from Amycolatopsis mediterranei (WO 98/10226; August et al. Chemistry and Biology (1998) δ: 69-79), soraphen from Sorangium cellulosum (S-A-5,716,849), and epothilones from Sorangium cellulosum (Tang et al. Science (2000) 287: 640-642).

WO 01/68867 discloses the complete DNA sequence of the gene cluster for the monensin type I polyketide synthase from S. cinnamonensis.

Genes that encode the type I PKS that governs the synthesis of the polyene pimaricin have been cloned from Streptomyces natalensis and the DNA sequence has been disclosed (Aparicio, J. et al. J. Biol. Chem. (1999) 274:10133-10139; Aparicio, J. et al. Chem. Biol. (2000) 7:895-905). The genes for the nystatin polyketide synthase have also been sequenced (Brautaset et al., Chem. Biol. (2000) 7: 395-403). Additionally, the cloning has been reported of the genes for the PKS for several other polyenes, including candicidin (Criado, L. M. et al. Gene (1993) 126: 135-139) and the heptaene polyene FR-008 (Hu, Z. et al. Mol. Microbiol. (1994) 14:163-172). However, so far there has been no report of the cloning, or cloning and DNA sequencing, of the genes for one of the most important polyenes, amphotericin.

Polyenes contain multiple asymmetric centres and are characterised by the presence of a large ring containing a cyclic hemiketal function, with a portion of the chain consisting of a conjugated polyene containing between three and eight conjugated trans C—C double bonds, and another portion of it consisting of a polyhydroxylated acyl chain. These structural features produce a characteristic shape which is well adapted for interaction with sterols in eukaryotic membranes, particularly with the ergosterol of fungal membranes. In addition, other groups that are often present include a free carboxyl group and a sugar residue, commonly D-mycosamine. Treatment of sensitive cells with inhibitory concentrations of polyenes results in collapse of the membrane potential and the loss of small molecules and ions especially potassium which leads to cell death (Omura, S. and Tanaka, H.: In Macrolide antibiotics, chemistry, biology and practice. (1984) Harcourt Bruce Jovanovich, Academic Press, New York.).

The utility of polyenes in the treatment of disease is well established. About 10 polyenes are used in clinical medicine, the most important are nystatin, candicidin, pimaricin and amphotericin B. Amphotericin B is the drug of choice to treat deep-seated systemic fungal infections (Georgopapadakou, N. H. and Walsh, T. J. Antimicrob. Agents and Chemotherapy (1996) 40:279-291). However its administration by intravenous or intrathecal routes is consistently associated with a number of toxic side-effects. Liposomal formulations of amphotericin B appear to display reduced toxicity (Abu-Salah, K. M. Brit. J. Biomed. Sci. (1996) 53: 122-133). MS-8209 is a derivative of amphotericin B which is more water soluble but retains activity (Saint-Julien, L. et al. Antimicrobial Agents Chemother. (1992) 36:2722-2728). There is an urgent need to develop derivatives of amphotericin which, while retaining or enhancing the efficacy of the parent molecule, are less affected by severe nephrotoxicity, insolubility, poor absorption and instability. Because of the structural complexity of polyenes, such novel analogues are not readily obtainable by total chemical synthesis, nor by chemical modifications of known polyketides.

In addition to their antifungal properties, amphotericin B and MS-8209 both showed activity in delaying the onset of symptoms associated with the transmissible spongiform encephalopathies scrapie and bovine spongiform encephalopathy (BSE). Polyenes may interfere with formation of abnormal forms of prion proteins during trafficking of sterol-rich membrane microdomains that contain these glycophosphatidyl-inositol-anchored proteins (Mange, A. et al., J. Neurochem. (2000) 74: 754-762). Both compounds prolonged the survival times of hamsters and mice infected intracerebrally with BSE or scrapie agents (Pocchiari, M. et al. J. Gen. Virol. (1987) 68:219-223; Adjou, K T. et al. Res. Virol. (1996) 147: 213-218). There is no known cure for the related human disease Creutzfeld-Jacob syndrome.

Amphotericin B inhibits infection of cultured cells by human immunodeficiency virus (HIV) (Schaffner, C. P. et al. Biochem. Pharmacol. 1986) 35: 4110-4113). The envelopes of these virus particles have a higher cholesterol: phospholipid ratio than host cell membranes (Aloia, R. C. et al. Proc. Natl. Acad. Sci. USA (1993) 90:5181-5185). MS-8209 has also been found to inhibit HIV-1 replication in vitro in all cell types without cytotoxicity and to restore T-cell activation via the CD3/TcR in HIV CD4+ cells (Cefai, D. et al. AIDS (1991) 5:1453-1461).

Amphotericin B is also active against Leishmania, a protozoal parasite that contains ergosterol precursors in its membranes (Hartsel, S., and Bolard, J. Trends Pharmacol. Sci. (1996) 17: 445-449).

The present invention provides a DNA sequence encoding all or part of the gene cluster for the biosynthesis of amphotericin as depicted in the appended sequence listings or an allele or mutation thereof. Also provided is the DNA sequence individually of one or more of amphG, amphH, amphDIII, amphI, amphJ, amphK, amphL, amphM, amphN, amphDII, amphDI, amphA, amphB and amphC as depicted in the appended sequence listing or an allele or mutation thereof.

The invention further provides a peptide encoded by any of the DNA sequences of the invention, the peptide being involved in the biosynthesis of amphotericin and having the amino acid sequence as set out in the appended sequence data or being a variant thereof having one of the activities set out below, namely:

| Gene | Activity | Seq. ID. No. |
|---|---|---|
| amphG | ABC transporter | 2 |
| amphH | ABC transporter | 3 |
| amphDIII | GDP - mannose dehydratase | 4 |
| amphI | Polyketide synthase modules 9 to 14 | 5 |
| amphJ | Polyketide synthase modules 15 to 17 | 6 |
| amphK | Polyketide synthase module 18 | 7 |
| amphL | Cytochrome P450 | 8 |
| amphM | Ferredoxin | 12 |
| amphN | Cytochrome P450 | 13 |
| amphDII | Sugar aminotransferase | 14 |
| amphDI | Glycosyl transferase | 15 |
| amphA | Polyketide synthase loading module | 16 |
| amphB | Polyketide synthase modules 1 and 2 | 17 |
| amphC | Polyketide synthase modules 3 to 8 | 18 |

Also is provided is a DNA sequence according to the invention encoding a single enzyme activity of a multienzyme encoded by any of amphA amphB, amphC, amphI, amphJ, amphK or a variant, mutant or part thereof, or encoding any one or more of the domains as set out in Table 3 or a variant or part thereof. Included is a DNA sequence which has a length of at least 30, preferably at least 60, bases.

The invention further provides a recombinant cloning or expression vector comprising a DNA sequence according to the invention and a transformant host cell transformed to contain a DNA sequence according to the invention and capable of expressing a peptide according to the invention. The invention also provides one or more recombinant vectors containing the DNA sequence encoding the amphotericin gene cluster or a portion thereof, in particular cosmids AMB3, AMC4, AMC31, AMC15 and/or AMC16 as described herein and as deposited respectively as transformants of E. coli XL 1-BLUE MR on Apr. 23, 2001 at National Collection of Industrial and Marine Bacteria, Aberdeen, Scotland, United Kingdom under the accession numbers NCIMB 41102, NCIMB 41103, NCIMB 41104, NCIMB 41105 and NCIMB 41106 respectively. The deposits were made under the Budapest Treaty on the International Recognition of the Deposit of Microorganisms for the Purposes of Patent Procedures. The deposits were made by the applicant University College Dublin, National University of Ireland, Dublin of Belfield, Dublin 4, Ireland.

The invention still further provides the use of a DNA sequence according to the invention in a method of preparing an amphotericin derivative or analogue antibiotic with altered properties.

The invention yet still further provides a hybridization probe comprising a DNA sequence according to the invention of a part thereof, including a polynucleotide which binds specifically to a region of the amphotericin gene cluster and in particular to a polynucleotide selected from amphDI, amphDII, amphL or amphN.

Also provided is the use of such a probe in a method of detecting the presence of a gene cluster which governs the synthesis of a polyene polyketide, and optionally isolating a gene cluster detected thereby. Further provided in the use of the probe in a method for identifying or isolating a gene or DNA sequence involved in the biosynthesis of a polyene polyketide.

Also provided is the use of a DNA sequence according to the invention in a method of preparing an amphotericin derivative or analogue antibiotic agent with altered properties.

Additionally provided is a cytochrome P450 enzyme encoded by amphL according to Seq.

ID. No. 8 or a derivative or variant thereof having hydroxylase activity, and a cytochrome P450 enzyme encoded by amphiN according to Seq. ID. No. 13 or a derivative or variant thereof having the ability to hydroxylate a methyl group to a hydroxymethyl group and hydroxymethyl group to a carboxyl group.

A portion of the amphotericin gene cluster according to the invention encoding a peptide having hydroxylase activity, preferably comprising amphL or amphN or a mutant, allele or other variant thereof encoding a polypeptide having hydroxylase activity can be used to provide a said activity in the biosynthesis of a polyketide other than amphotericin.

Further provided is a DNA sequence comprising DNA encoding at least one PKS loading module and a plurality of PKS extension modules, and which can be expressed to produce a polyketide, wherein at least one of the said extension modules or at least one domain thereof is an amphotericin extension module or domain or a variant thereof and is contiguous to a further one of said extension modules or a domain to which it is not naturally contiguous. In one arrangement said further modules or domain includes an amphotericin module or domain or variant thereof. In another arrangement said further modules or domain includes a module or domain of a PKS of a polyketide other than amphotericin or a variant thereof. Said loading module is conveniently adapted to load a starter unit other than a starter unit normally received by the adjacent extension module.

Also provided is a polyketide synthase encoded by the DNA sequence of the invention and a polyketide compound produced by such a synthase.

The invention further provides the use of a portion of the amphotericin gene cluster encoding ER5 of amphC as defined in Table 3 and Seq. ID. No. 18 for inactivation of amphotericin A production leading to production of amphotericin B substantially uncontaminated by amphotericin A, and use of a portion of the amphotericin gene cluster encoding ER5 of amphC as defined in Table 3 and Seq. ID. No. 18 to engineer the biosynthesis of a mixture of two classes of polyketide products which differ in having either methylene or enoyl groups at corresponding defined positions. Also provided is the use of amphDIII or amphDII or amphDI mutants for production of amphotericin derivatives glycosylated with alternative sugars, and use of the amphDIII or amphDH gene sequences in engineered biosynthesis of perosaminyl-amphoteronolide B. Further provided is the use of the amphDIII or amphDII and amphN gene sequence in engineered biosynthesis of perosaminyl-16-descarboxyl-16-methyl amphoteronolide B. Yet still further provided is the use of the amphDIII, amphDII and amphDI gene sequence for preparing polypeptides capable of the addition of mycosamine to a polyketide other than amphoteronolide A or amphoteronolide B.

The invention also provides the novel compounds 8-deoxyamphotericin B, 8-deoxyamphotericin A, 8-deoxyamphoteronolide B, and 8-deoxyamphoteronolide A. Further provided is the use of the amphDIII, amphDII and amphDI gene sequences for preparing polypeptides for in vitro synthesis of GDP-mycosamine.

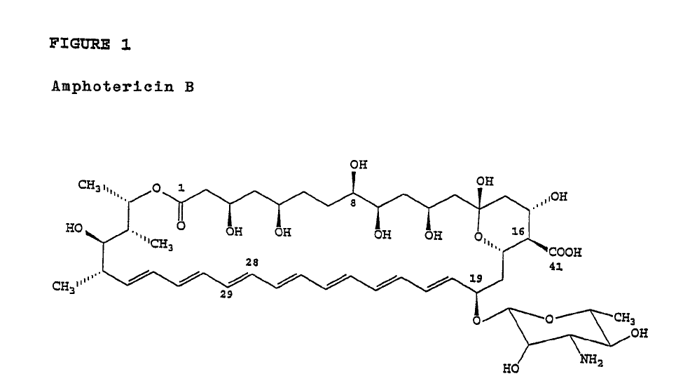

Amphotericins A and B are produced by the actinomycete Streptomyces nodosus. Amphotericin B, the more active form, has the structure shown in FIG. 1. Amphotericin A differs from amphotericin B only in that the C28-C29 double bond is reduced. Structure-activity studies based on chemical modification (Cheron, M. et al. Biochem. Pharmacol. (1988) 37: 827-836) have shown that the positively-charged amino group on the sugar is essential for antifungal activity; that the absence or masking of the C-16 carboxyl group increases the selectivity of the polyene interaction with the target ergosterol over the unwanted interaction with cholesterol (although it enhances neurotoxicity); and that the modification of the C13 hemiketal is associated with an amelioration of side-effects on human cells (Taylor et al. J. Antibiotics (1992) 46:486-493). There is a need for new methods of modifying the structure of amphotericin, nystatin, pimaricin, candicidin and other polyenes to produce even greater selectivity towards the membranes of the pathogenic fungi that cause mycoses such as invasive pulmonary aspergillosis, mucosal candidiasis, cryptococcal meningitis, disseminated histoplasmosis and coccidiomycosis (Georgopapadakou, N. H. and Walsh, T. J. Antimicrob. Agents and Chemotherapy (1996) 40:279-291)

Although the structures of polyenes differ significantly from those of other complex polyketides such as the polyhydroxylated macrolides or the polyethers, their biosynthesis appears to take place by a metabolic pathway which has many common elements. Thus experiments using carbon 13-labelled precursors have shown that amphotericin B is synthesised from sixteen acetate and three propionate residues (McNamara, C. M. et al. J. Chem. Soc., Perkin Trans. I (1998) 83-87). The hydroxylation at C-8, the oxidation of the C-41 methyl group and the attachment of mycosamine at the C-19 hydroxy group are proposed to occur as late steps in the biosynthesis, after formation of the polyene macrolactone, but the relative timing of these modifications is unknown.

The present invention provides a DNA sequence comprising the amphotericin gene cluster.

Some embodiments of the invention will now be described by way of example with reference to the accompanying drawings in which:

FIG. 1 shows the structure of amphotericins;

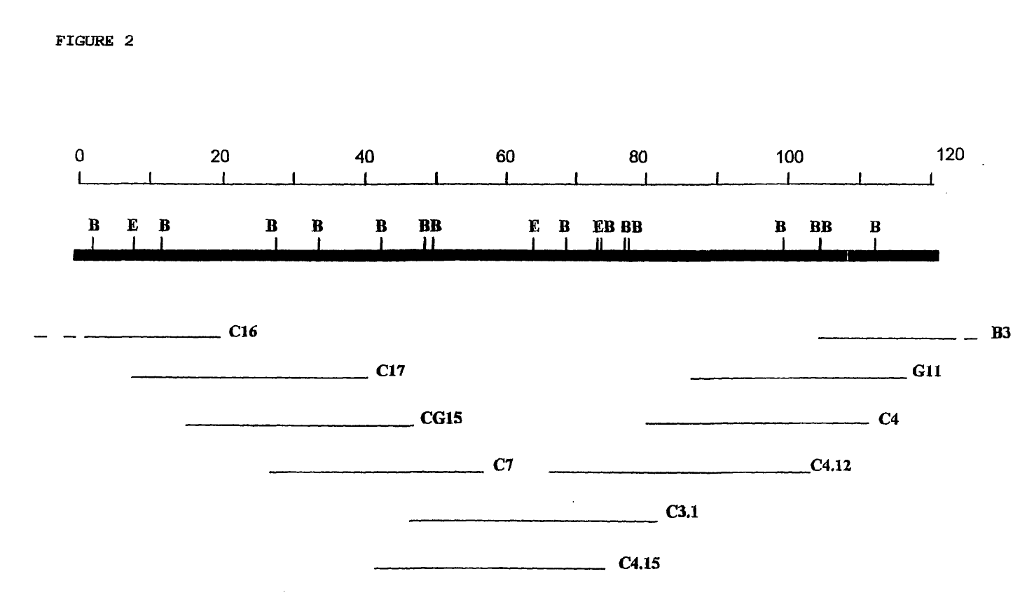

FIG. 2 shows overlapping cosmid clones representing the amphotericin biosynthetic gene cluster and showing Eco RI(E) and Bam HI(H) restriction sites;

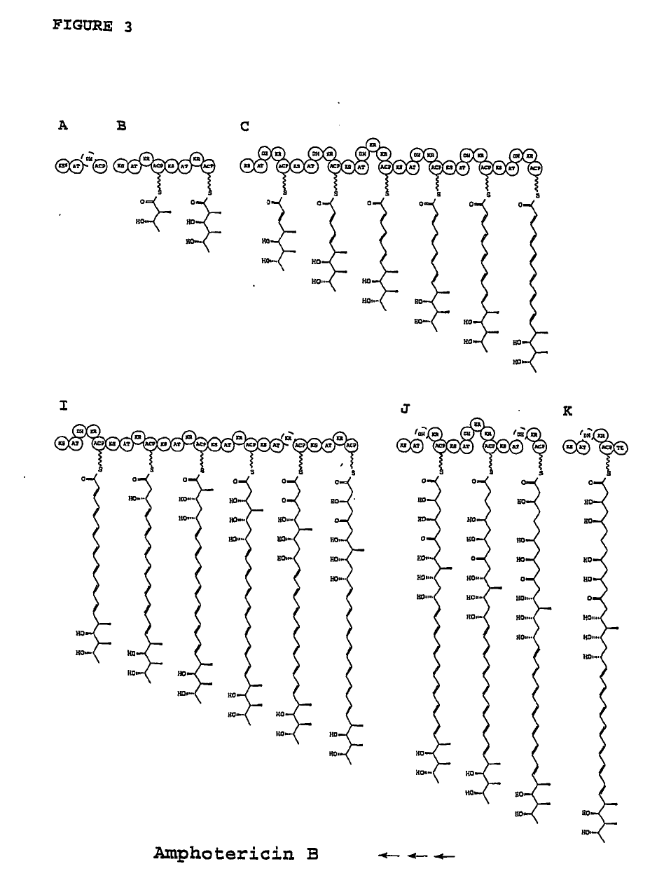

FIG. 3 illustrates the organisation of the amphotericin PKS enzyme complex;

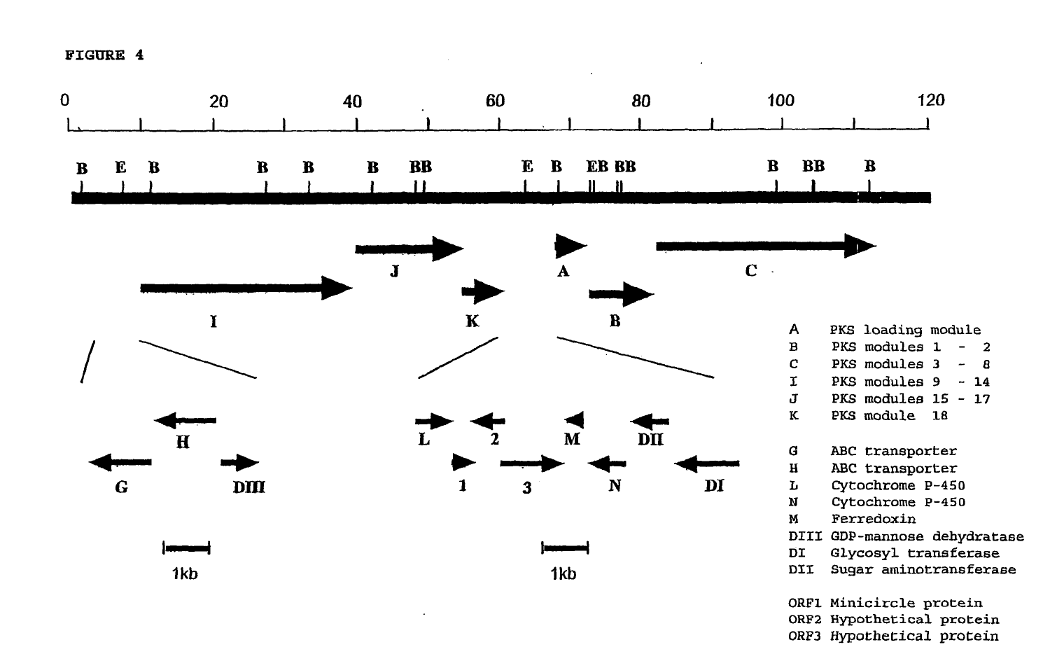

FIG. 4 illustrates the organisation of the amphotericin biosynthetic genes;

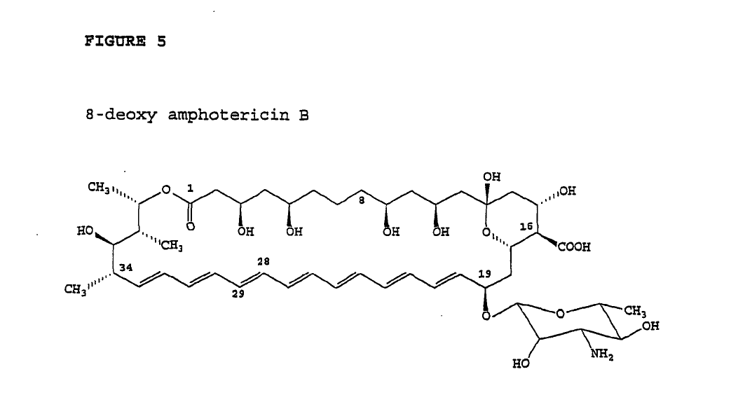

FIG. 5 shows the structure of 8-deoxy amphotericin B;

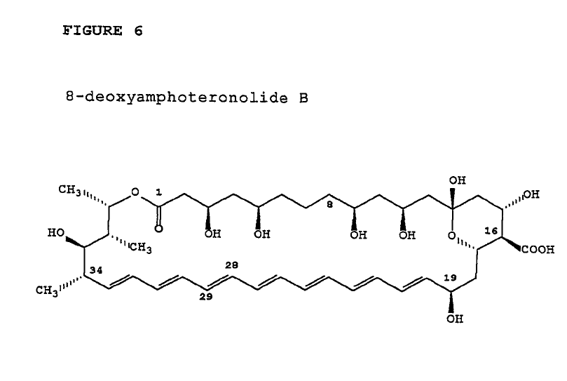

FIG. 6 shows the structure of 8-deoxyamphoteronolide B; and

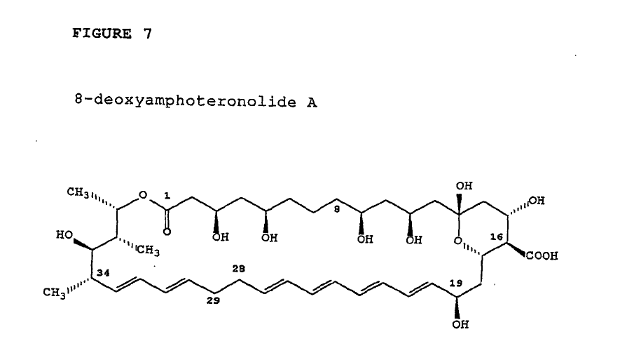

FIG. 7 shows the structure of 8-deoxyamphoteronolide A.

Table 1 lists the content of the appended nucleotide sequence of the amphotericin biosynthetic gene cluster;

Table 2 lists the content of the appended amino acid sequences of proteins encoded by this cluster; and

Table 3 lists the genes and shows the extents of coding sequences for domains and proteins within the cluster.

We have found that the overall gene organization of the amphotericin biosynthetic gene cluster (FIG. 4) is similar to that previously found for many macrolide biosynthetic gene clusters, which have one or more open reading frames (ORFs) encoding large multifunctional PKSs flanked by other genes which encode functions required for the biosynthesis of the antibiotic. In the case of amphotericin, there is the unusual feature of two six-module PKS proteins; but there is again a separate module of enzymes for each cycle of polyketide chain extension, exactly as found for modular PKSs for macrolide biosynthesis. Thus there are 18 condensations predicted to be required for the production of the carbon skeleton of amphotericin, and in agreement with this there are found to be 18 modules of PKS enzymes distributed among the 6 PKS ORFs (see FIG. 3). The interdomain region preceding the ER domain in module 5 is shorter than the corresponding regions in the complete reduction loops in other PKSs. This probably constrains movement of this domain in cycle 5 so that the precursors of amphotericins A and B are produced by the PKS. This is a rare example of a lapse in PKS programming fidelity that results from a partially active reduction domain.

An additional feature of the PKS of the amphotericin cluster is an unusual mechanism of chain initiation. The amphA gene encodes a loading module with the domain structure organisation KS^{S}-AT-DH-ACP. The direct linkage of a DH to an ACP domain is unusual.

The AT domain has the signature sequence characteristic of a malonyl transferase (Haydock et al., FEBS Lett (1995) 374: 246-248) and probably loads malonyl groups onto the ACP domain. The KS^{S} domain has a serine residue in place of the active site cysteine.

This domain may act as a decarboxylase that acts on malonyl-ACP to generate acetyl starter units.

KS domains are converted to potent decarboxylases when glutamine (Q) is present in place of the active site cysteine (Witkowska, A., et al. (1999) Biochemistry 38: 11643-11650). KS^{Q} enzymes appear in loading modules for some other macrolide PKSs. Like the KS-β components of aromatic PKSs, KS^{Q} domains decarboxylate malonyl or methylmalonyl groups to acetyl or propionyl starter units. This may represent an efficient means of delivering primers to the first KS and may also allow for stricter control of starter unit selection. It is uncertain whether KS^{S} domains are equally efficient at generating primers. The active site cysteine-161 of the KS domain of rat fatty acid synthase has been replaced with various amino acids. A cysteine-serine change gave a mutant enzyme that retained a low residual condensation activity and had only a weak decarboxylase activity (Witkowska, A., et al. (1999) Biochemistry 38: 11643-11650). A weak KS^{S} decarboxylase may provide primers at an adequate rate for synthesis of the amphotericin polyketide. The DH domain in AmphA is presumably redundant since it would not normally encounter a β-hydroxyacyl-ACP substrate.

Multiple uses of portions of the cloned and sequenced DNA from the amphotericin cluster will readily occur to the person skilled in the art. This DNA is useful in the engineering of mutant strains of S. nodosus for the high level production of either natural or novel recombinant polyketides. The availability of the complete sequence allows domain, multi-domain or module swaps to be performed. For example, the AT domain that specifies the methylmalonyl-CoA extender unit in module 11 of the amphotericin PKS can be replaced by an acetate-specific AT domain to eliminate the methyl branch at C-16 and lead to synthesis of an analogue lacking a carboxyl group. Similarly the alteration of the level of reduction in a module, by manipulation of the reductive enzymes, can be applied to the amphotericin genes. For example, the extremely desirable elimination of the production of the less active amphotericin A can be accomplished by suitable modification of the reductive loop in module 5. GB 9814622.8 describes in detail a particularly flexible method for accomplishing these modifications by swapping of entire sets of reductive domains obtained usually from natural PKSs and containing a different complement of active reductive domains, either DH-ER-KR, or DH-KR, or KR, or none.

The amphotericin PKS is one of the largest for which a sequence is available. This system will allow engineered biosynthesis of libraries of novel large macrolide compounds. In general the targetted alteration of the pattern of substitution of side chains or reduction level along the polyketide chain produced by the amphotericin PKS will lead to altered polyketide products. It is possible, by provision of a suitable thioesterase at the C-terminus of one of the internal extension modules of the amphotericin PKS, together with provision of an appropriately placed hydroxy group earlier in the chain, to produce novel macrolide products from this polyene PKS system, or alternatively novel polyenes of defined chain length and chosen ring size. Novel macrolides can also be produced by fusing a loading module, from the amphotericin, erythromycin or avermectin PKSs, to internal extension modules of the amphotericin PKS. Domains or modules from the amphotericin PKS could be incorporated into other PKS systems to allow production of useful new compounds.

In addition to the PKS genes the amphotericin cluster also contains genes responsible for post-polyketide modifications. Manipulation of these late genes could also result in biosynthesis of valuable amphotericin analogues.

The presence of the amphDIII gene for a GDP-mannose 4,6 dehydratase was surprising since it had previously been postulated that mycosamine was synthesised from dTDP-glucose (Martin, J. F. (1984) In Macrolide Antibiotics: Chemistry, Biology and Practice, (1984) Academic Press Inc., Harcourt Brace Jovanovich Publishers, New York; Stockmann, M. and Piepersberg, W. FEMS Microbiology Lett. (1992) 90:185-190). The sequence data suggest that the biosynthetic pathway to mycosamine (3, 6 dideoxy-3-amino D mannose) involves isomerisation of GDP-6-deoxy-4-keto-mannose to GDP-6-deoxy-3-keto-mannose followed by aminotransfer to give GDP-mycosamine. This final step is probably catalysed by the AmphDII protein which is similar to aminotransferases involved in biosynthesis of perosamine (4, 6 dideoxy-4-amino mannose). The amphDI gene encodes a glycosyltransferase that adds mycosamine to the aglycone core of amphotericin. It is remarkable that the amphotericin cluster does not contain a gene that is likely to encode a GDP-6-deoxy-4-keto-mannose 3, 4 isomerase. It has been suggested that the eryCII gene encodes a dTDP-6-deoxy-4-ketoglucose 3,4 isomerase that functions in the biosynthesis of desosamine (Salah-Bey et al., Mol. Gen. Genet. (1998) 257, 542-553). Homologous genes have been found in clusters for other macrolides that are glycosylated with desosamine, mycaminose or daunosamine (Hallis, T. M., and Liu, H.-W. Acc. Chem. Res. (1999) 32, 579-588). Biosynthesis of all of these amino sugars involves 3,4 isomerisation of dTDP-6-deoxy-4-ketoglucose. These putative isomerases are similar to cytochrome P450 enzymes but lack the conserved cysteine residue that co-ordinates the haem iron. No homologous gene is present in the amphotericin cluster. Significantly perhaps, non-enzymatic ketoisomerisation of dTDP-6-deoxy-4-keto-glucose to dTDP-6-deoxy-3-ketoglucose has been observed in vitro (Naundorf and Klaffke, Carbohydrate Research (1996) 285, 141-150). The reaction is apparently catalysed by a basic Dowex 2-X8 anion exchange resin and goes to completion after 10 hours incubation at 4° C. In mycosamine biosynthesis, it is possible that GDP-6-deoxy-4-ketomannose can isomerise to GDP-6-deoxy-3-ketomannose in the absence of a conventional enzyme.

Overexpression of the AmphDII protein could allow in vitro synthesis of GDP-mycosamine from GDP-6-deoxy-4-ketomannose, which is readily available. A non-enzymatic catalyst like a Dowex anion-exchange resin might be used to catalyse the necessary ketoisomerisation step. S. nodosus mutants with disrupted PKS genes should still express the amphDIII, amphDII and amphDI genes, and could be used for addition of mycosamine to other aglycones. The amphDIII, amphDII and amphDI genes could be expressed in an alternative host for similar biotransformation of other aglycones.

In addition to glycosylation, oxygenation reactions occur at C-8 and C-41 in the amphotericin precursor. The amphL and amphN genes encode cytochrome P450 enzymes. It would be impossible to predict the precise roles of these enzymes from sequence data alone. However, disruption of amphL gives a mutant S. nodosus strain that synthesises 8-deoxyamphotericins A and B (vide infra). This shows that the AmphL protein is responsible for hydroxylation at C-8. The presence of only one other P450 gene suggests that a single enzyme, AmphN, converts the C41 methyl group first to a CH₂OH group and then to a carboxyl group. This was surprising since it might have been expected that at least two P450s would be required for formation of the exocyclic carboxyl group. Inactivation of amphN would lead to biosynthesis of a highly desirable amphotericin analogue with a methyl branch in place of the exocyclic carboxyl group.

Disruption of the amphDIII gene gave a recombinant S. nodosus strain that produces the aglycones 8-deoxyamphoteronolide A and 8-deoxyamphoteronolide B. Taken together, these results indicate that in the preferred order of post-polyketide modifications, the carboxyl group is formed first, glycosylation occurs next and finally hydroxylation occurs at C-8. Attempts to disrupt the amphn gene were unsuccessful, suggesting that accumulation of a completely unmodified macrolactone ring might be detrinental to the producing cell.

The amphDI and amphDII genes could be used as hybridisation probes to clone the genes for GDP-perosamine synthase and perosaminyl transferase from Streptomyces aminophilus, the producer of the aromatic heptaene perimycin. Experimentation is required to replace the S. nodosus chromosomal amphDI and amphDII genes with the genes for GDP-perosamine synthase and perosaminyl transferase. Expression of these genes under the control of the amphDI promoter could result in engineered biosynthesis of analogues that are glycosylated with perosamine (perosaminyl amphoteronolide B or perosaminyl-16-methyl-16-descarboxylamphoteronolide B).

In S. nodosus cells, GDP-perosamine synthase would intercept GDP-6-deoxy-4-ketomannose, prior to 3,4 isomerisation, to generate GDP-perosamine. The S. aminophilus glycosyl transferase should perosaminylate an early amphotericin precursor that is structurally similar to the perimycin aglycone in the region of the glycosylation site. The aglycone of perimycin has a methyl branch in place of the exocyclic carboxyl group. The S. aminophilus glycosyl transferase would therefore be expected to perosaminylate the amphotericin macrolactone ring prior to formation of the carboxyl group. This could allow subsequent non-lethal disruption of amphN leading to production of the highly desirable analogue perosaminyl-16-methyl-16-descarboxylamphoteronolide B.

When tested against Saccharomyces cerevisiae 8-deoxyamphotericin B was found to have an antifungal activity as great as that of amphotericin B. The utility of 8-deoxyamphotericin B and other analogues can be tested further to allow assessment of commercial value. The 8-deoxyamphoteronolide aglycone shows no antifungal activity, but could be used for glycosylation engineering experiments. This aglycone compound could be fed to a streptomycete capable of synthesising alternative activated sugars like the amino sugar dTDP-mycaminose, or the neutral sugar dTDP-mycarose. Mutagenesis and DNA shuffling of the amphDI gene could be carried out to generate glycosyl transferases capable of recognising the amphotericin aglycone and alternative (d)NDP-sugars. These genes would be introduced into the streptomycete strain. Strains capable of adding alternative amino sugars would be detected by screening for antifingal activity. Addition of the disaccharide mycarosyl-mycaminose onto 8-deoxyamphoteronolides A and B could restore antifungal activity and increase water-solubility.

Gene disruption and replacement rely on homologous recombination between engineered DNA and chromosomal sequences. Introduction of DNA into S. nodosus by standard methods was surprisingly difficult. Attempts based on protoplast transformation, conjugation or electroporation were unsuccessful. Gene disruption could be achieved using phage transduction using recombinant KC515 phage to inject engineered DNA. However, this method was inefficient and laborious because isolation of even small quantities of KC5S15 vector DNA from phage particles is technically difficult.

To facilitate genetic engineering of S. nodosus and other Streptomyces species, a novel bifunctional vector was constructed from phage KC515 and the E. coli plasmid pACYC177 (Chang, A. C. Y., and Cohen, S. N., J. Bacteriol (1978) 134: 1141-1156) (GenBank accession number=X 06402). This plasmid was chosen because it has a low copy number and does not introduce direct repeat sequences in the bifunctional vector.

These features make the bifunctional vector stable. A fragment of pACYC177 containing the plasmid p15A origin of replication and the kanamycin resistance gene was ligated to KC515 DNA to create the bifunctional vector KC UCD 1. This was propagated in E. coli XL-1 Blue MR and milligram quantities of pure KC-UCD1 DNA were isolated by standard plasmid isolation procedures. This can be used for efficient construction of recombinant phages for gene disruption and replacement in streptomycetes.

### EXAMPLE 1

Cloning of the Amphotericin Biosynthetic Gene Cluster Using DNA Probes Derived from the Erythromycin Biosynthetic Genes of Saccharopolyspora erythraea.

A cosmid library was constructed from genomic DNA of amphotericin-producing Streptomyces nodosus ATCC 14899 using standard methods (Hopwood, D. A. et al. Genetic manipulation of Streptomyces. A laboratory manual. (1985) Norwich. John Innes Foundation; Sambrook, J. et al. Molecular Cloning. A laboratory manual. 2nd ed. (1989) Cold Spring Harbour Laboratory Press, New York). High molecular weight genomic DNA was partially digested with Sau 3A and fragments in the size range 35 to 40 kb were isolated by sucrose density gradient centrifugation. These fragments were cloned into the cosmid vector pWE15 (Evans, G. A. et al. Gene (1989) 79: 9-20), previously digested with Bam HI and treated with alkaline phosphatase. The DNA was packaged and the resulting library was propagated on Escherichia coli strain XL1-Blue MR (Stratagene) cells. Colonies were screened by hybridisation with a DNA probe derived from the gene for 6 deoxyerythronolide B synthase 2 (Bevitt, D. J. et al. Eur. J. Biochem. (1992) 204:39-49). Cosmid DNA was purified from positively-hybridising clones and characterised by restriction analysis. The presence of polyketide synthase genes was confirmed by sequencing the ends of restriction fragments subcloned from these cosmids.

### EXAMPLE 2

Sequencing of the Biosynthetic Gene Cluster for Amphotericin.

Five cosmids obtained by screening the library as in Example 1 were used to obtain the entire sequence of the amphotericin biosynthetic gene cluster. These cosmids AM.B3, AM.C4, AM.C31, AM.C15, AM.C16 (see FIG. 2) between them contain the entire DNA of the cluster and of the adjacent regions of the chromosome. They have been deposited under the Budapest Treaty at National Collection of Industrial and Marine Bacteria (NCIMB), 23 St. Machair Drive, Aberdeen AB243RY, United Kingdom under the NCIMB accession numbers 41102 (AM.B3), 41103 (AM.C4), 41104 (AM.C31), 41105 (AM.C15), 41106 (AM.C16) on April 23, 2001.

The DNA of each cosmid was separately subjected to partial digestion with Sau 3A and fragments of approximately 1.5 to 2.0 kb were separated by agarose gel electrophoresis. The fragments were then ligated into the plasmid vector pBC SK+ (Stratagene), previously digested with Bam HI and treated with alkaline phosphatase. The libraries were transformed into E. coli XL1-Blue MR and plated on 2TY agar medium containing chloramphenicol (50 μg/ml) to select for plasmid-containing cells. Plasmid DNA was purified from individual transformants and sequenced using the Sanger dye-terminator procedure on an ABI 377 automated sequencer (Sanger, F. Science (1981) 214: 1205-1201). The sequence data obtained from single random subclones of a cosmid was assembled into a single continuous sequence and edited using GAP4.1 program of the STADEN gene analysis package (Staden, R. Molecular Biotechnology (1996) δ: 233-241).

The sequence is set out in the appended sequence listing identified as Seq. ID. No. 1.

### EXAMPLE 3

Inactivation of the Amphotericin Biosynthetic Gene Cluster.

Chromosomal gene disruption experiments were used to verify the identity of the cloned polyketide synthase gene cluster. A 5 kb Bam HI fragment of cosmid 4 was subcloned into pUC118 and partial sequencing was carried out. A 3.8 kb sub-fragment extending from a Bgl II site in the insert to the Pst I site in the pUC118 polylinker was excised and subcloned between the Bam HI and Pst I sites of KC515. This 3.8 kb region is internal to the amphC gene and contains much of the coding sequence for module 7. The recombinant phage, KC515-M7, was used to infect S. nodosus and lysogens were obtained by selecting for the thiostrepton resistance gene within the prophage DNA. Genomic DNA from a typical lysogen was digested with several restriction enzymes and analysed by

Southern hybridisation using labelled 3.8 kb fragment as a probe. This revealed that the phage had integrated into the polyketide synthase gene. The disruption mutant was designated S. nodosus DM7.

The disruption mutant was grown on FDS medium (fructose 20 g/l, dextrin 60 g/l, soya flour 30 g/l, CaCO₃ 10 g/l, (pH 7.0)) with good aeration at 28° C. Samples were taken at intervals and supernatants were assayed for amphotericins by bioassay using Saccharomyces cerevisiae NCYC006 as an indicator organism. Amphotericin production was also monitored by UV spectrophotometry (McNamara et al. (1998) J. Chem. Soc. Perkin Trans. 1 1998: 83-87). Samples of the culture were mixed with nine volumes of dimethyl sulphoxide and sonicated for 20 minutes. The extract was centrifuged and the supernatant fraction was diluted with nine volumes of methanol. The absorption spectrum was measured in the range 260 to 450 nm. Amphotericin B shows four specific absorption maxima at wavelengths 346, 364, 382 and 405 nm. Amphotericin A absorbs at 280, 292, 305 and 320 nm. No trace of amphotericin was detected in cultures of the disruption mutant either by bioassay or by spectrophotometry. Amphotericin was detected by both methods in parallel control cultures of S. nodosus ATCC14899.

### EXAMPLE 4

Construction of a Bifunctional Vector KC-UCD1 from Phage KC515 and the E. coli Plasmid pACYC184

The 2.9 kb Bam HI-Pst I fragment of pACYC177 contains the plasmid p15A origin of replication and the kanamycin resistance gene (Chang, A. C. Y., and Cohen, S. N., J. Bacteriol (1978) 134: 1141-1156). This fragment was ligated between the Bam HI and Pst I sites of phage KC515 DNA and the ligated DNA was introduced into Streptomyces lividans 1326 by transfection. Recombinant phage plaques were identified by PCR using oligonucleotide primers APR101 [5′ ACGGGAAACGTCTTGCTCGA 3′] and APR201 [5′CATGAGTGACGACTGAATCC 3′] specific for the kanamycin resistance gene. Recombinant phage gave a 575 base pair product. A typical recombinant phage was designated KC-UCD1. Genomic DNA was isolated from this phage and introduced into competent Escherichia coli XL-1 Blue MR cells. Transformants were selected on kanamycin agar. Milligram quantities of KC-UCD1 DNA were isolated from E. coli by standard plasmid isolation procedures.

### EXAMPLE 5

Use of Bifunctional Vector KC UCD1 for Targeted Disruption of an Amphotericin Biosynthetic Gene

A 2.0 kb Kpn I fragment of cosmid 17 was subcloned into pUC118. Sequence analysis using universal and reverse primers indicated that a 1660 bp Bgl II-Pst I fragment of this plasmid was internal to the amphI gene and encoded part of module 9. This fragment was subcloned between the Bam HI and Pst I sites of KC-UCD 1. The recombinant phage, KC UCD 1-M9, was used to infect S nodosus and thiostrepton-resistant lysogens were selected. Analysis of genomic DNA from a typical lysogen indicated that integration of a phage had disrupted the polyketide synthase gene. The resulting mutant was designated S. nodosus DM9.

The disruption mutant was tested for amphotericin production as described in Example 3. No trace of amphotericin was detected either by bioassay or by spectrophotometry.

### EXAMPLE 6

Targeted Disruption of the amphL Gene for a Cytochrome P450 Enzyme.

The sequence revealed two genes for cytochrome P450 enzymes that function in modification of the macrolactone ring. These enzymes are required for hydroxylation at C8 and oxidation of C41 to a carboxyl group. The 1059 bp Sac I fragment internal to the amphL gene was subcloned between the Sac I sites of KC515. The recombinant phage was propagated on S. nodosus and thiostrepton-resistant lysogens were selected. Chromosomal DNA from a typical lysogen was analysed by Southern hybridisation using the labelled 1059 bp Sac I fragment as a probe. This confirmed that the phage had integrated into the chromosomal amphL gene.

The resulting strain S. nodosus DP450-1 was grown on FDS medium. Polyenes were extracted from the cultures using butanol. Analysis by electrospray mass spectrometry in negative ion mode revealed that the major products had masses (−H⁺) of 906 and 920. Analysis using positive ion mode revealed the same products with masses (+Na⁺) of 930 and 944. These compounds were identified as 8-deoxy amphotericin B (FIG. 5) and an analogue with a propionate starter unit 8-deoxy C37 desmethyl-C37 ethyl amphotericin B. In repeat experiments 8-deoxy amphotericin A and 8-deoxy C37 desmethyl-C37 ethyl amphotericin A were also detected.

### EXAMPLE 7

Targeted Disruption of the amphDIII Gene for GDP-Mannose Dehydratase.

A 2092 bp region containing the amphDIII gene was amplified by PCR using oligonucleotide primers MC1 [5′CCG AGGATCC CGC ACC AGA TGC AAA ACG AC 3′] and MC2 [5′ TAA ACT GCA GGA CAG CAC GCT GCC GGT GTT G 3′ ]. The product was cloned into plasmid pUC118. A Bgl II site within the amphDIII gene sequence was filled in to create a frameshift mutation. The mutated fragment was excised with Bam HI and Pst I and cloned into KC515. The recombinant phage was propagated on S. nodosus and lysogens were obtained by selecting for thiostrepton resistance. A typical lysogen was cultured in the absence of thiostrepton to allow excision of the prophage DNA by a second recombination event. Protoplasts were prepared and allowed to regenerate. Individual colonies were screened for thiostrepton sensitivity resulting from excision of the prophage by a second homologous recombination event. Replacement of the amphDIII gene with the mutated copy would result in loss of the Bgl II site from the chromosomal DNA. This region was amplified from several revertants by PCR using oligonucleotides MC1 and MC2 as primers. The PCR products were digested with either Bgl II or Kpn I. Several amphDIII mutants were identified as strains giving PCR products that were not digested with Bgl II. Control digestions showed that all PCR products were readily digested by Kpn I.

A typical mutant, designated S. nodosus D3, was grown on FDS medium. Polyenes were exracted from the culture using chloroform-methanol. LTV spectrophotometry showed that both heptaene and tetraene compounds were present. Analysis by electrospray mass spectrometry in negative ion mode gave major products with molecular masses of 761 and 763, indicating that 8-deoxyamphoteronolides B and A were being produced (FIGS. 6 and 7 respectively).

### REFERENCES

1. Abu-Salah, K. M. (1996) Amphotericin B: an update. British Journal of Biomedical Science, 53, 122-133.
2. Aloia, R. C., Tian, H., and Jensen, F. C. (1993) Lipid composition and fluidity of the human immunodeficiency virus envelope and host cell plasma membranes. Proceedings of the National Academy of Sciences of the United States of America, 90, 5181-5185.
3. Adjou, K. T., Demaimay, R., Lasmezas, C. I., Seman, M., Deslys, J.-P. and Dormont, D. (1996) Differential effects of a new amphotericin B derivative, MS-8209, on mouse BSE and scrapie: implications for the mechanism of action of polyene antibiotics. Research in Virology, 147, 213-218.
4. Aparicio, J. F., Colina, A., Ceballos, E. and Martin, J. F. (1999) The biosynthetic gene cluster for the 26-membered ring polyene macrolide pimaricin. Journal of Biological Chemistry, 274, 10133-10139.
5. Aparicio, J. F., Fouces, R., Mendes, M. V., Olivera, N. and Martin, J. (2000) A complex multienzyme system encoded by five polyketide synthase genes is involved in the biosynthesis of the 26-membered polyene macrolide pimaricin in Streptomyces nodosus. Chemistry and Biology, 7, 895-905
6. August, P. R., Tang, L., Yoon, Y. J., Ning, S., Muller, R., Yu, T. W., Taylor, M., Hoffmann, D., Kim, C. G., Zhang, X. H., Hutchinson, C. R. and Floss, H. G. (1998) Biosynthesis of the ansamycin antibiotic rifamycin: deductions from the molecular analysis of the rif biosynthetic gene cluster of Amycolatopsis mediterranei S699. Chemistry & Biology, 5, 69-79.
7. Bartel, P. L., Zhu, C. B., Lampel, J. S., Dosch, D. C., Connors, N. C., Strohl, W. R., Beale, J. M. and Floss, H. G. (1990) Biosynthesis of anthraquinones by interspecies cloning of actinorhodin biosynthesis genes in &treptomycetes—clarification of actinorhodin gene functions. Journal of Bacteriology, 172, 4816-4826.
8. Bevitt, D. J., Cortes, J., Haydock, S. F., and Leadlay, P. F. (1992) 6-Deoxyerythronolide synthase 2 from Saccharopolyspora erythraea. Cloning of the structural gene, sequence analysis and inferred domain structure of the multifunctional enzyme. European Journal of Biochemistry, 204, 3949.
9. Bibb, M. J., Biro, S., Motamedi, H., Collins, J. F. and Hutchinson, C. R. (1989) Analysis of the nucleotide sequence of the Streptomyces glaucescens Tcm genes provides key information about the enzymology of polyketide antibiotic biosynthesis. EMBO Journal, 8, 2727-2736.
10. Bibb, M. J. and Hopwood, D. A. (1981) Genetic studies of the fertility plasmid SCP2 and its SCP2* variants in Streptomyces coelicolor A3(2). Journal of General Microbiology, 126, 427-442.
11. Bisang, C., Long, P. F., Cortes, J., Staunton, J. and Leadlay, P. F. (1999) A chain initiation factor common to both modular and aromatic polyketide synthases. Nature, 401, 502-505.
12. Böhm, I., Holzbaur, I. E., Hanefeld, U., Cortes, J., Staunton, J., Leadlay, P. F. (1998) Engineering of a minimal modular polyketide synthase and targeted alteration of the stereospecificity of polyketide chain extension. Chemistry and Biology, 5,407-412.

13. Brautaset, T., Sekurova, O. N., Sletta, H., Ellingsen, T. E., Strom, A. R., Valla, S. and Zotchev, S. B. (2000) Biosynthesis of the polyene antifungal antibiotic nystatin in Streptomyces noursei ATCC 11455: analysis of the gene cluster and deduction of the biosynthetic pathway. Chemistry and Biology, 7, 395-403.
14. Chang, A. C. Y., and Cohen, S. N. (1978) Journal of Bacteriology, 134, 1141-1156.
15. Cefai, D., Hadida, F. and Jung, M. (1991) MS-8209, a new amphotericin B derivative that inhibits HIV-1 replication in vitro and restores T-cell activation via the CD3/TcR in HIV-infected CD4+ cells. AIDS, 5, 1453-1461
16. Cheron, M., Cybulska, B., Mazerski, J., Gryzbowska, J., Czerwinski, A. and Borowski, E. (1998) Quantitative structure-activity relationships in amphotericin B derivatives. Biochemical Pharmacology, 37,827-836
17. Cortés, J., Haydock, S. F., Roberts, G. A., Bevitt, D. J. and Leadlay, P. F. (1990) An unusually large multifunctional polypeptide in the erythromycin producing polyketide synthase of Saccharopolyspora erythraea. Nature, 348, 176-178.
18. Cortés, J., Wiesmann, K. E. H., Roberts, G. A., Brown, M. J. B., Staunton, J. and Leadlay, P. F. (1995) Repositioning of a domain in a modular polyketide synthase to promote specific chain cleavage. Science, 268, 1487-1489.
19. Criado, L. M., Martin, J. F. and Gil, J. A. (1993) The pab gene of Streptomyces griseus, encoding p-aminobenzoic acid synthase is located between genes possibly involved in candicidin biosynthesis. Gene, 126, 135-139.
20. Davis, N. K. and Chater, K. F. (1990) Spore color in Streptomyces coelicolor A3(2) involves the developmentally regulated synthesis of a compound biosynthetically related to polyketide antibiotics. Molecular Microbiology, 4, 1679-1691.
21. Donadio, S., McAlpine, J. B., Sheldon, P. J., Jackson, M. and Katz, L. (1993) An erythromycin analog produced by reprogramming of polyketide synthesis. Proceedings of the National Academy of Sciences of the United States of America, 90, 7119-7123.
22. Donadio, S., Staver, M. J., McAlpine, J. B., Swanson, S. J. and Katz, L. (1991) Modular organization of genes required for complex polyketide biosynthesis. Science, 252, 675-679.
23. Evans, G. A., Lewis, K. and Rothenberg, B. E. (1989) High efficiency vectors for cosmid microcloning and genomic analysis. Gene, 79, 9-20.
24. Fernandez-Moreno, M. A., Caballero, J. L., Hopwood, D. A. and Malpartida, F. (1991) The Act cluster contains regulatory and antibiotic export genes, direct targets for translational control by the bldA transfer-RNA gene of Streptomyces. Cell, 66, 769-780.
25. Fernandez-Moreno, M. A., Martinez, E., Boto, L., Hopwood, D. A. and Malpartida, F. (1992) Nucleotide sequence and deduced functions of a set of cotranscribed genes of Streptomyces coelicolor A3(2) including the polyketide synthase for the antibiotic actinorhodin. Journal of Biological Chemistry, 267, 19278-19290.
26. Georgopapadakou, N. H. and Walsh, T. J. (1996) Antifungal agents: chemotherapeutic targets and immunologic strategies. Antimicrobial Agents and Chemotherapy, 40, 279-291.
27. Gramajo, H. C., Takano, E. and Bibb, M. J. (1993) Stationary phase production of the antibiotic actinorhodin in Streptomyces coelicolor A3(2) is transcriptionally regulated. Molecular Microbiology, 7, 837-845.
28. Hallam, S. E., Malpartida, F. and Hopwood, D. A. (1988) Nucleotide sequence, transcription and deduced function of a gene involved in polyketide antibiotic synthesis in Streptomyces coelicolor. Gene, 74, 305-320.
29. Hallis T. M., and Liu, H. W. (1999) Learning nature's strategies for making deoxy sugars: pathways, mechanisms and combinatorial applications. Accounts Chemical Research 32, 579-588.
30. Hartsel, S. and Bolard, J. (1996) Amphotericin B: new life for an old drug. Trends in Pharmacological Sciences 17, 445-449.
31. Haydock, S. F., Aparicio, J. F., Molnar, I., Schwecke, T., Konig, A., Marsden, A. F. A., Galloway, I. S., Staunton, J., and Leadlay, P. F. (1995) Divergent sequence motifs correlated with the substrate specificity of methyl)malonyl-CoA: acyl carrier protein transacylase domains in modular polyketide synthases. FEBS Letters 374, 246-248.
32. Hitchman, T. S., Crosby, J., Byrom, K. J., Cox, R. J. And Simpson, T. J. (1998) Catalytic self-acylation of type II polyketide synthase acyl carrier proteins. Chemistry and Biology 5, 35-47.
33. Hopwood, D. A., Bibb, M. J., Chater, K. F., Kieser, T., Bruton, C. J., Kieser, H. M., Lydiate, D. J., Smith, C. P., Ward, J. M. and Schrempf, H. (1985) Genetic manipulation of Streptomyces, a laboratory manual. John Innes Institution, Norwich, UK.
34. Hopwood, D. A, and Sherman, D. H. (1990) Molecular genetics of polyketides and its comparison to fatty acid biosynthesis. Annual Reviews of Genetics 24, 37-66.
35. Hopwood, D. A. (1997) Genetic contributions to understanding polyketide synthases. Chemical Reviews 97, 2465-2478
36. Hu, Z., Bao, K., Zhou, X., Zhou, Q., Hopwood, D. A., Kieser, T. and Deng, Z. (1994) Repeated polyketide synthase modules involved in biosynthesis of a heptaene macrolide by Streptomyces sp. FR-008. Molecular Microbiology, 14, 163-172.
37. Hutchinson, C. R. and Fujii, I. (1995) Polyketide synthase gene manipulation—a structure function approach in engineering novel antibiotics. Annual Review of Microbiology, 49, 201-238.
38. Kakavas, S. J., Katz, L. and Stassi, D. (1997) Identification and characterization of the niddamycin polyketide synthase genes from Streptomyces caelestis. Journal of Bacteriology, 179, 7515-7522.
39. Kao, C. M., Luo, G. L., Katz, L., Cane, D. E. and Khosla, C. (1995) Manipulation of macrolide ring size by directed mutagenesis of a modular polyketide synthase. Journal of the American Chemical Society, 117, 9105-9106.
40. Kao, C. M., McPherson, M., McDaniel, R. N., Fu, H., Cane, D. E., Khosla, C. (1998) Journal of the American Chemical Society, 117,9105-9106.
41. Khosla, C., Gokhale, R. S., Jacobsen, J. R., and Cane, D. E. (1999) Tolerance and specificity of polyketide synthases. Annu. Rev. Biochem. 68: 219-253.
42. McNamara, C., Box, S., Crawforth, J. M., Hickman, B. S., Norwood, T. J. and Rawlings, B. J. (1998) Biosynthesis of amphotericin B. Journal of the Chemical Society Perkin Transactions 1,1998,83-87.
43. MacNeil, D. J., Occi, J. L., Gewain, K. M., MacNeil, T., Gibbons, P. H., Ruby, C. L. and Danis, S. J. (1992) Complex organization of the Streptomyces avermitilis genes encoding the avermectin polyketide synthase. Gene, 115, 119-125.
44. Mange, A., Milhavet, O., McMahon, H. E. M., Casanova, D. and Lehman, S. (2000) Effect of amphotericin B on wild-type and mutated prion proteins in cultured cells: Putative mechanism of action in transmissible spongiform encephalopathies. J. Neurochem. 74, 754-762.
45. Marsden, A. F. A., Caffrey, P., Aparicio, J. F., Loughran, M. S., Staunton, J. And Leadlay, P. F. (1994) Stereospecific acyl transfers on the erythromycin-producing polyketide synthase. Science 263, 378-380.
46. Marsden, A. F. A., Wilkinson, B., Cortés, J., Dunster, N. J., Staunton, J. and Leadlay, P. F. (1998) Engineering broader specificity into an antibiotic-producing polyketide synthase. Science, 279, 199-202.
47. Martin, J. F. (1984) Biosynthesis, regulation and genetics of polyene macrolide antibiotics. pp405-424 In Macrolide Antibiotics: Chemistry, Biology and Practice, Omura, S. ed. Academic Press Inc. Harcourt Brace Jovanovich Publishers, New York.
48. Naundorf, A. and Klaffke, W. (1996) substrate specificity of native dIDP-D-glucose-4,6-dehydratase: chemo-enzymatic syntheses of artificial and naturally occurring deoxy sugars. Carbohydrate Research 285: 141-150.
49. Omura, S. and Tanaka, H. (1984) Production structure and antifungal activity of polyene macrolides. pp351-404 In Macrolide Antibiotics: Chemistry, Biology and Practice, Omura, S. ed. Academic Press Inc. Harcourt Brace Jovanovich Publishers, New York.
50. Pocchiari, M., Schmittinger, S. and Masullo, C. (1987) Amphotericin B delays the incubation period of scrapie in intracerebrally inoculated hamsters. Journal of General Virology, 68, 219-223.
51. Revill, W. P., Bibb, M. J., and Hopwood, D. A. (1995) Purification of a malonyl transferase from Streptomyces coelicolor A3(2) and analysis of its genetic determinant. Journal of Bacteriology 177, 3946-3952.
52. Rodicio, M. R., Bruton, C. J., Chater, K. F. (1985) New derivatives of the Streptomyces temperate phage ΦC31 useful for the cloning and functional analysis of Streptomyces DNA. Gene 34,283-292.
53. Salah-Bey, K. Doumith, M., Michel, J.-M., Haydock, S., Cortes, J., Leadlay, P. F. And Raynal, M. C. (1998) Targeted gene inactivation for the elucidation of deoxysugar biosynthesis in the erythromycin producer Saccharopolyspora erythraea Molecular & General Genetics 257, 542-553.
54. Sambrook, J., Fritsch, E. F. and Maniatis, T. (1989) Molecular cloning: a laboratory manual. 2nd ed. Cold Spring Harbor Laboratory Press, New York.
55. Sanger, F. (1981) Determination of nucleotide sequences in DNA. Science, 214, 1205-1210.
56. Saint-Julien, L., Joly, V., Seman, M., Carbon, C. and Yeni, P. (1992) Activity of MS-8209, a nonester amphotericin B derivative, in treatment of experimental systemic mycoses. Antimicrobial Agents and Chemotherapy, 36, 2722-2728.
57. Schaffner, C. P., Plescia, O. J., Pontani, D., Sun, D., Thornton, A., Pandey, R. C., Sarin, P. S. (1986) Anti-viral activity of amphotericin B methyl ester: inhibition of HTLV-III replication in cell culture. Biochemical Pharmacology 35: 4110-4113.
58. Schwecke, T., Aparicio, J. F., Molnár, I., König, A., Khaw, L. E., Haydock, S. F., Oliynyk, M., Caffrey, P., Cortés, J., Lester, J. B., Böhm, G. A., Staunton, J. and Leadlay, P. F. (1995) The biosynthetic gene cluster for the polyketide immunosuppressant rapamycin. Proceedings of the National Academy of Sciences of the United States of America, 92, 7839-7843.
59. Sherman, D. H., Malpartida, F., Bibb, M. J., Kieser, H. M. and Hopwood, D. A. (1989) Structure and deduced function of the granaticin-producing polyketide synthase gene cluster of Streptomyces violaceoruber Tu22. EMBO Journal, 8, 2717-2725.
60. Staden, R. (1996) The Staden sequence analysis package. Molecular Biotechnology, 5,233-241.
61. Stassi, D., Kakavas, S. J., Reynolds, K. A., Gunawardana, G., Swanson, S., Zeidner, D., Jackson, M., Liu, H., Buko, A., and Katz, L. (1998) Ethyl-substituted erythromycin derivatives produced by directed metabolic engineering. Proceedings of the National Academy of Sciences of the United States of America, 95, 7305-7309.
62. Stockmann, M. and Piepersberg, W. (1992) Gene probes for the detection of 6-deoxyhexose metabolism in secondary metabolite-producing streptomycetes. FEMS Microbiology Letters, 90,185-190.
63. Swan, D. G., Rodriguez, A. M., Vilches, C., Mendez, C. and Salas, J. A. (1994) Characterization of a Streptomyces antibioticus gene encoding a type I polyketide synthase which has an unusual coding sequence. Molecular & General Genetics, 242, 358-362.
64. Tang, L., Shah, S., Chung, L., Carney, J., Katz, L., Khosla, C. and Bryan, J. (2000) Cloning and heterologous expression of the epothilone gene cluster. Science, 287, 640-642.
65. Taylor, A. W., Costello, B., Hunter, P. A., McLachlan, W. S. and Shanks, C. T. (1992) Synthesis and antifungal selectivity of new derivatives of amphotericin B modified at the C13 position. Journal of Antibiotics, 46, 486-493.

66. Witkowska, A., Joshi, A. K., Lindqvist, Y., and Smith, S. (1999) Conversion of a β-ketoacyl synthase to a malonyl decarboxylase by replacement of the active site cysteine with glutamine. Biochemistry, 38, 11643-11650.

TABLE 1

| Sequence | Appended Seq. ID. No. |
|---|---|
| Nucleotide sequence of the amphotericin | Seq. ID. No. 1 |
| biosynthetic gene cluster | |

TABLE 2
Amino acid sequences of proteins encoded by
amphotericin biosynthetic gene cluster

| | Appended |
|---|---|
| Sequence | Seq. ID. No. |
| AmphG, ABC transported. Length: 606 | Seq. ID. No. 2 |
| AmphH, ABC transporter. Length: 607 | Seq. ID. No. 3 |
| AmphDIII, GDP-mannose dehydratase. Length: 345 | Seq. ID. No. 4 |
| AmphI, Polyketide synthase multienzyme housing | Seq. ID. No. 5 |
| extension modules 9, 10, 11, 12, 13 and 14. Length: | |
| 9511 | |
| AmphJ, Polyketide synthase multienzyme housing | Seq. ID. No. 6 |
| extension modules 15, 16 and 17. Length: 5644 | |
| AmphK, Polyketide synthase multienzyme housing | Seq. ID. No. 7 |
| extension module 18 and thioesterase. Length: 2035 | |
| AmphL, Cytochrome P450. Length: 397 | Seq. ID. No. 8 |
| ORF1, Hypothetical protein. Length: 170 | Seq. ID. No. 9 |
| ORF2, Hypothetical protein. Length: 285 | Seq. ID. No. 10 |
| ORF3, Hypothetical protein. Length: 534 | Seq. ID. No. 11 |
| AmphM, Ferredoxin. Length: 66 | Seq. ID. No. 12 |
| AmphN, Cytochrome P450. Length: 400 | Seq. ID. No. 13 |
| AmphDII, NDP-sugar aminotransferase. Length: 353 | Seq. ID. No. 14 |
| AmphD1, Glycosyl transferase. Length: 484 | Seq. ID. No. 15 |
| AmphA, Polyketide synthase multienzyme housing | Seq. ID. No. 16 |
| loading module. Length: 1413 | |
| AmphB, Polyketide synthase multienzyme housing | Seq. ID. No. 17 |
| extension modules 1 and 2. Length: 3191 | |
| AmphC, Polyketide synthase multienzyme housing | Seq. ID. No. 18 |
| extension modules 3, 4, 5, 6, 7 and 8. Length: 10918 | |

TABLE 3

| Gene | Function | Start | End |
|---|---|---|---|
| AmphG | ABC transporter | 1824 | 4 |
| AmphH | ABC transporter | 3721 | 1805 |
| AmphDIII | GDP-mannose dehydratase | 3840 | 4872 |
| AmphI | Polyketide synthase modules 9-14 | 5042 | 33572 |
| | KS9 | 5144 | 6379 |
| | AT9 malonate specific | 6725 | 7747 |
| | DH9 | 7808 | 8329 |
| | KR9 | 9245 | 9784 |
| | ACP9 | 10040 | 10297 |
| | KS10 | 10357 | 11620 |
| | AT10 malonate-specific | 11941 | 12973 |
| | KR10 | 13867 | 14413 |
| | ACP10 | 14678 | 14935 |
| | KS11 | 14990 | 16252 |
| | AT11 methylmalonate-specific | 16592 | 17617 |
| | KR11 | 18473 | 19021 |
| | ACP11 | 19286 | 19593 |
| | KS12 | 19604 | 20869 |
| | AT12 | 21191 | 22225 |
| | KR12 | 23090 | 23638 |
| | ACP12 | 23900 | 24156 |
| | KS13 | 24219 | 25467 |
| | AT13 malonate-specific | 25763 | 26790 |
| | ACP13 | 28534 | 28794 |
| | KS14 | 28852 | 30090 |
| | AT14 malonate-specific | 30416 | 31417 |
| | KR14 | 32279 | 32830 |
| | ACP14 | 33088 | 33345 |
| amphJ | Polyketide synthase modules 15-17 | 33584 | 50518 |
| | KS15 | 33701 | 34969 |
| | AT15 | 35337 | 36335 |
| | DH15 | 36383 | 36904 |
| | KR15 | 37835 | 38392 |
| | ACP15 | 38657 | 38914 |
| | KS16 | 38978 | 40234 |
| | AT16 malonate-specific | 40502 | 41509 |
| | DH16 | 41514 | 42088 |
| | ER16 | 43115 | 43978 |
| | KR16 | 43991 | 44598 |
| | ACP16 | 44813 | 45070 |
| | KS17 | 45137 | 46390 |
| | AT17 malonate-specific | 46673 | 47680 |
| | DH17 (inactive) | 47738 | 48220 |
| | KR17 | 49196 | 49753 |
| | ACP17 | 50003 | 50254 |
| amphK | Polyketide synthase module 18 | 50571 | 56675 |
| | KS18 | 50670 | 51932 |
| | AT18 malonate-specific | 52221 | 53237 |
| | DH18 | 53295 | 53810 |
| | KR18 | 54720 | 55276 |
| | ACP18 | 55518 | 55784 |
| | TE | 55908 | 56675 |
| amphL | Cytochrome P450 | 56829 | 58019 |
| ORF1 | Hypothetical protein | 58139 | 58648 |
| ORF2 | Hypothetical protein | 59610 | 58756 |
| ORF3 | Hypothetical protein | 59887 | 61470 |
| amphM | Ferredoxin | 61995 | 61798 |
| amphN | Cytochrome P450 | 63250 | 62051 |
| AmphDII | Sugar aminotransferase | 64308 | 63247 |
| AmphDI | Glycosyl transferase | 65862 | 64324 |
| amphA | Polyetide synthase loading module | 66081 | 70319 |
| | KS-L | 66105 | 67362 |
| | AT-L | 67794 | 68810 |
| | DH-L (inactive) | 68853 | 69383 |
| | ACP-L | 69816 | 70073 |
| amphB | Polyketide synthase modules 1-2 | 70366 | 79938 |
| | KS-1 | 70483 | 71746 |
| | AT1 methylmalonate-specific | 72082 | 73107 |
| | KR1 | 73948 | 74502 |
| | ACP1 | 74770 | 75027 |
| | KS2 | 75088 | 76365 |
| | AT2 methylmalonate-specific | 76699 | 77733 |
| | KR2 | 78604 | 79152 |
| | ACP2 | 79426 | 79683 |
| amphC | Polyketide synthase modules 3-8 | 79956 | 112709 |
| | KS3 | 80061 | 81314 |
| | AT3 malonate-specific | 81600 | 82604 |
| | DH3 | 82662 | 83189 |
| | KR3 | 84105 | 84662 |
| | ACP3 | 84927 | 85175 |
| | KS4 | 85242 | 86504 |
| | AT4 malonate-specific | 86826 | 87830 |
| | DH4 | 87891 | 88415 |
| | KR4 | 89328 | 89879 |
| | ACP4 | 90150 | 90398 |
| | KS5 | 90459 | 91721 |
| | AT5 malonate-specific | 92046 | 93050 |
| | DH5 | 93108 | 93635 |
| | ER5 | 94668 | 95528 |
| | KR5 | 95544 | 96101 |
| | ACP5 | 96357 | 96614 |
| | KS6 | 96681 | 97943 |
| | AT6 malonate-specifc | 98346 | 99290 |
| | DH6 | 99348 | 99875 |
| | KR6 | 100782 | 101333 |
| | ACP6 | 101589 | 101846 |
| | KS7 | 101907 | 103169 |
| | AT7 malonate-specific | 103506 | 104513 |
| | DH7 | 104571 | 105101 |
| | KR7 | 106032 | 106589 |
| | ACP7 | 106881 | 107141 |
| | KS8 | 107205 | 108467 |
| | AT8 malonate-specific | 108810 | 109814 |
| | DH8 | 109875 | 110402 |
| | KR8 | 111381 | 111938 |
| | ACP8 | 112197 | 112448 |

It will of course be understood that the present invention is not limited to the specific details described above, which are given by way of example only, and that various modifications and alternations are posible without departing from the scope of the invention as defined in the appended claims.

1. A DNA sequence comprising at least part of the sequence of an amphotericin gene cluster as set out in the appended sequence listing Seq. ID. No. 1.

2. A DNA sequence according to claim 1 comprising the complete amphotericin gene cluster or a variant thereof.

3. A DNA sequence encoding at least part of at least one polypeptide which is necessary for the biosynthesis of amphotericin, and which is encoded by DNA included in the appended sequence listing Seq. ID. No. 1 or an allele, mutation or other variant thereof.

4. A DNA sequence according to claim 3 which comprises at least part of one or more of the following genes: amphDI, amphDII, amphL or amphN.

5. A DNA sequence according to claim 4 comprising all of the genes listed therein or an allele, mutation or other variant thereof.

6. A DNA sequence according to claim 3 encoding at least part of one or more of the polypeptides set out below, said polypeptide having the amino acid sequence as set out in the appended sequence data or being a variant thereof having the specified activity:

| Peptide | Activity | Seq. ID. No. |
|---|---|---|
| AmphG | ABC transporter | 2 |
| AmphH | ABC transporter | 3 |
| AmphDIII | GDP-mannose dehydratase | 4 |
| AmphI | Polyketide synthase modules 9 to 14 | 5 |
| AmphJ | Polyketide synthase modules 15 to 17 | 6 |
| AmphK | Polyketide synthase module 18 | 7 |
| AmphL | Cytochrome p450 | 8 |
| amphM | Ferredoxin | 12 |
| amphN | Cytochrome p450 | 13 |
| amphDII | Sugar aminotransferase | 14 |
| amphDI | Glycosyl transferase | 15 |
| amphA | Polyketide synthase loading modules | 16 |
| amphB | Polyketide synthase modules 1 and 2 | 17 |
| amphC | Polyketide synthase modules 3 to 8 | 18 |

7. A DNA sequence according to claim 6 encoding a single enzyme activity of a multienzyme encoded by any of amphA amphB, amphC, amphI, amphJ, amphK or a variant, mutant or part thereof.

8. A DNA sequence according to claim 1 encoding any one or more of the domains as set out in Table 3 or a variant or part thereof.

9. A DNA sequence according to claim 1 which has a length of at least 30, preferably at least 60, bases.

10. A recombinant cloning or expression vector comprising a DNA sequence according to claim 1.

11. A transformant host cell which has been transformed to contain a DNA sequence according to claim 1 and which is capable of expressing a corresponding polypeptide.

12. A hybridisation probe comprising a DNA sequence according to claim 1.

13. Use of a probe according to claim 12 to detect a polyene PKS cluster, optionally followed by isolation of the detected cluster.

14. Use of a probe according to claim 12 which encodes at least part of a polypeptide having a known function to detect genes encoding polypeptides having analogous function.

15. A hybridisation probe according to claim 12 which binds to a region of the amphotericin gene cluster and in particular to a polynucleotide selected from amphDI, amphDII, amphL or amphN.

16. Use of a DNA sequence according to claim 1 in a method of preparing an amphotericin derivative or analogue antibiotic agent with altered properties.

17. A cytochrome P450 enzyme encoded by amphL according to Seq. ID. No. 8 or a derivative or variant thereof having hydroxylase activity.

18. A cytochrome P450 enzyme encoded by amphN according to Seq. ID. No. 13 or a derivative or variant thereof having the ability to hydroxylate a methyl group to a hydroxymethyl group and hydroxymethyl group to a carboxyl group.

19. Use of a portion of the amphotericin gene cluster according to claim 1 encoding a peptide having hydroxylase activity, preferably comprising amphL or amphN or a mutant, allele or other variant thereof encoding a polypeptide having hydroxylase activity to provide a said activity in the biosynthesis of a polyketide other than amphotericin.

20. A DNA sequence comprising DNA encoding at least one PKS loading module and a plurality of PKS extension modules, and which can be expressed to produce a polyketide, wherein at least one of the said extension modules or at least one domain thereof is an amphotericin extension module or domain or a variant thereof and is contiguous to a further one of said extension modules or a domain to which it is not naturally contiguous.

21. A DNA sequence according to claim 20 wherein said further modules or domain includes an amphotericin module or domain or variant thereof.

22. A DNA sequence according to claim 20 wherein said further modules or domain includes a module or domain of a PKS of a polyketide other than amphotericin or a variant thereof.

23. A DNA sequence according to claim 20 wherein said loading module is adapted to load a starter unit other than a starter unit normally received by the adjacent extension module.

24. A polyketide synthase encoded by the DNA sequence of claims of 20 to 23 claim 20.

25. A polyketide compound produced by a synthase according to claim 24.

26. Use of a portion of the amphotericin gene cluster encoding ER5 of amphC as defined in Table 3 and Seq. ID. No. 18 for inactivation of amphotericin A production leading to production of amphotericin B substantially uncontaminated by amphotericin A.

27. Use of a portion of the amphotericin gene cluster encoding ER5 of amphC as defined in Table 3 and Seq. ID. No. 18 to engineer the biosynthesis of a mixture of two classes of polyketide products which differ in having either methylene or enoyl groups at corresponding defined positions.

28. Use of amphDIII or amphDII or amphDI mutants for production of amphotericin derivatives glycosylated with alternative sugars.

29. Use of the amphDIII or amphDII gene sequences in engineered biosynthesis of perosaminyl-amphoteronolide B.

30. Use of the amphDIII or amphDII and amphN gene sequence in engineered biosynthesis of perosaminyl-16-descarboxyl-16-methyl amphoteronolide B.

31. Use of the amphDIII, amphDII and amphDI gene sequence for preparing polypeptides capable of the addition of mycosamine to a polyketide other than amphoteronolide A or amphoteronolide B.

32. 8-deoxyamphotericin B.

33. 8-deoxyamphotericin A.

34. 8-deoxyamphoteronolide B.

35. 8-deoxyamphoteronolide A.

36. Use of the amphDIII, amphDII and amphDI gene sequences for preparing polypeptides for in vitro synthesis of GDP-mycosamine.

37. A DNA sequence according to claim 2 encoding any one or more of the domains as set out in Table 3 or a variant or part thereof.

38. A DNA sequence according to claim 3 encoding any one or more of the domains as set out in Table 3 or a variant or part thereof.

39. A DNA sequence according to claim 4 encoding any one or more of the domains as set out in Table 3 or a variant or part thereof.

40. A DNA sequence according to claim 5 encoding any one or more of the domains as set out in Table 3 or a variant or part thereof.

41. A DNA sequence according to claim 6 encoding any one or more of the domains as set out in Table 3 or a variant or part thereof.

42. A DNA sequence according to claim 7 encoding any one or more of the domains as set out in Table 3 or a variant or part thereof.

43. A DNA sequence according to claim 2 which has a length of at least 30, preferably at least 60, bases.

44. A DNA sequence according to claim 3 which has a length of at least 30, preferably at least 60, bases.

45. A DNA sequence according to claim 4 which has a length of at least 30, preferably at least 60, bases.

46. A DNA sequence according to claim 5 which has a length of at least 30, preferably at least 60, bases.

47. A DNA sequence according to claim 6 which has a length of at least 30, preferably at least 60, bases.

48. A DNA sequence according to claim 7 which has a length of at least 30, preferably at least 60, bases.

49. A DNA sequence according to claim 8 which has a length of at least 30, preferably at least 60, bases.

50. A recombinant cloning or expression vector comprising a DNA sequence according to claim 2.

51. A recombinant cloning or expression vector comprising a DNA sequence according to claim 3.

52. A recombinant cloning or expression vector comprising a DNA sequence according to claim 4.

53. A recombinant cloning or expression vector comprising a DNA sequence according to claim 5.

54. A recombinant cloning or expression vector comprising a DNA sequence according to claim 6.

55. A recombinant cloning or expression vector comprising a DNA sequence according to claim 7.

56. A recombinant cloning or expression vector comprising a DNA sequence according to claim 8.

57. A recombinant cloning or expression vector comprising a DNA sequence according to claim 9.

58. A transformant host cell which has been transformed to contain a DNA sequence according to claim 2 and which is capable of expressing a corresponding polypeptide.

59. A transformant host cell which has been transformed to contain a DNA sequence according to claim 3 and which is capable of expressing a corresponding polypeptide.

60. A transformant host cell which has been transformed to contain a DNA sequence according to claim 4 and which is capable of expressing a corresponding polypeptide.

61. A transformant host cell which has been transformed to contain a DNA sequence according to claim 5 and which is capable of expressing a corresponding polypeptide.

62. A transformant host cell which has been transformed to contain a DNA sequence according to claim 6 and which is capable of expressing a corresponding polypeptide.

63. A transformant host cell which has been transformed to contain a DNA sequence according to claim 7 and which is capable of expressing a corresponding polypeptide.

64. A transformant host cell which has been transformed to contain a DNA sequence according to claim 8 and which is capable of expressing a corresponding polypeptide.

65. A transformant host cell which has been transformed to contain a DNA sequence according to claim 9 and which is capable of expressing a corresponding polypeptide.

66. A hybridisation probe comprising a DNA sequence according to claim 2.

67. A hybridisation probe comprising a DNA sequence according to claim 3.

68. A hybridisation probe comprising a DNA sequence according to claim 4.

69. A hybridisation probe comprising a DNA sequence according to claim 5.

70. A hybridisation probe comprising a DNA sequence according to claim 6.

71. A hybridisation probe comprising a DNA sequence according to claim 7.

72. A hybridisation probe comprising a DNA sequence according to claim 8.

73. A hybridisation probe comprising a DNA sequence according to claim 9.

74. Use of a probe according to claim 66 to detect a polyene PKS cluster, optionally followed by isolation of the detected cluster.

75. Use of a probe according to claim 67 to detect a polyene PKS cluster, optionally followed by isolation of the detected cluster.

76. Use of a probe according to claim 66 which encodes at least part of a polypeptide having a known function to detect genes encoding polypeptides having analogous function.

77. Use of a probe according to claim 67 which encodes at least part of a polypeptide having a known function to detect genes encoding polypeptides having analogous function.

78. A hybridisation probe according to claim 66 which binds to a region of the amphotericin gene cluster and in particular to a polynucleotide selected from amphDI, amphDII, amphL or amphN.

79. A hybridisation probe according to claim 67 which binds to a region of the amphotericin gene cluster and in particular to a polynucleotide selected from amphDI amphDII, amphL or amphN.

80. Use of a DNA sequence according to claim 2 in a method of preparing an amphotericin derivative or analogue antibiotic agent with altered properties.

81. Use of a DNA sequence according to claim 3 in a method of preparing an amphotericin derivative or analogue antibiotic agent with altered properties.

82. Use of a DNA sequence according to claim 4 in a method of preparing an amphotericin derivative or analogue antibiotic agent with altered properties.

83. Use of a DNA sequence according to claim 5 in a method of preparing an amphotericin derivative or analogue antibiotic agent with altered properties.

84. Use of a DNA sequence according to claim 6 in a method of preparing an amphotericin derivative or analogue antibiotic agent with altered properties.

85. Use of a DNA sequence according to claim 7 in a method of preparing an amphotericin derivative or analogue antibiotic agent with altered properties.

86. Use of a DNA sequence according to claim 8 in a method of preparing an amphotericin derivative or analogue antibiotic agent with altered properties.

87. Use of a DNA sequence according to claim 9 in a method of preparing an amphotericin derivative or analogue antibiotic agent with altered properties.

88. A DNA sequence according to claim 21 wherein said loading module is adapted to load a starter unit other than a starter unit normally received by the adjacent extension module.

89. A DNA sequence according to claim 22 wherein said loading module is adapted to load a starter unit other than a starter unit normally received by the adjacent extension module.

90. A polyketide synthase encoded by the DNA sequence of claim 21.

91. A polyketide synthase encoded by the DNA sequence of claim 22.


SEQUENCE LISTING (1 sequence)

SEQ ID NO: 1 - DNA, 113193 bp, Streptomyces nodosus
cggtcagccgaccgtcacatcggcggcgggcgcgtcggcgcgggcggcgtgcagccgggcgtaggctccctgggcgtcgatgagctggtcatgggtgccctgctcgacgatccggccggacttcatcacgacgatgacatcggcgtccctgatggtggacagacggtgcgcgatcacaaagctcgtccggcccggccgcagggtattcatggcccgctggatcagcagctcggtacgggtgtccacggagctggtcgcctcgtccaggacgagcacggccggccgggcgaggaacgccctcgcgaccgtgatcagctgcttctccccggcgctgacaccgcccgactcgtcgtccaggaccgtgtcgtacccctgcgacagggtgcggatgaaccggtcggcgtaggtcgcgcgggccgcctccacgatgtcggcgcgggtcgcgcccggggacccgtaggcgatgttctcggcgatcgtgcccttgaacagccaggtgtcctggaggacgaggccgaagcgggaccgcaggtcgtcgcggttcatcgcggcggtgtcggtgccgtccagcaggatgcggcccgagtcgatctcgtagaagcgcatcagcagattggcgaccgtggtcttgccggcgccgctcggtccgacgaccgccaccgtgctgccgggttccacggacagcgaaagaccctcgatgagcggggtgtcgggggagtagcggaacgacacgtcctggaactccacccggccctcgacgcgttccaccgcgagcgggcggtccggctcgggttcctgctccggggcgtccagcagcgtgaacacccgctgcgcggaggccaccccggactggaggcggcccgcgaccgaggcgatctccacgatcggctggctgaactgccgtgcgtacaggatgaacgcctgcacatcgcccagcgtcagtgagccgttgacgaccttccaggcgccgacgacggcgaccgcgacatagccgaggttggcgacgaacatcatcacgggctcgatcgcgccggacgcgaactgcgcccccgaaccggcccggtacaccgcctcgttgcgctcgtcgaagcgctgctcggcctccgcccgccggtcgaaggccttgaccagcgcgtgcccggcgcacatctcctcgacatgcgcggtcagcttgccgctcgcggaccactgggccgcgtactgcggctgggcccgcttgctgatccaggcggcgagcagccccgacaccgggacgctcagcagcatcaccccggccagcgagggcgagatgatcagcatcagggacaacatgatgaccagcgagaacaccgagctgatcagatcggtcagggtctgctggagggtctgctggaggttgtcgacgtcgtccgtgccgcggctgagcgtctcgcccgccggcttccggtcgaagtagcgcagcggcagccgcgccagcttctcctgcgcccggtgccgcaggtcgtggatgagcaggcggaccaccgaggccaccagccagttctggaccagcatgaacaccgatgccaccacatacagcaccagcaggatcatcagcagtctgccgagcgccccgaagtcgacgcccggggccggtccgccgtgcccgaggacgccgtcggcgatcaggtcggtgacccggcccagcagcagcgggccgaaggcgttgaggaggatgccgacgacacccgccgtgagggcgaggcccaccgaccggcggtgcgggcgcagcagagcggcgagccggcggaccggctgctccgactcccgctcctcttcgtgctcctcaggaacgtccggcgccatgagcggtttcctctcgggtgcgctgggagagcgcgatctcgcggtaggtgggactgtcgcgcagcagcgcgtcatgggtgccggtggcggcgatgccgccctggtccaggaggacgatccggtcggcgtcgcgcacggaggcgacgcgctgcgccacggtgatcacggtggcgccctcggtgtagggcaccagggccttccgtagcgccgcctccgtgctctggtcgagcgccgagaaggagtcgtcgaagagatagatggcggggcggcgcagcagcgcccgtgcgatcgccagccgctggcgctgaccgccggagacggtggtgccgccctgagcgatctccgtgtcgaggccgccgagccgggtcacgaagccttcgtgctgggcgatccgcagcacctcccacagctcctcgtccgtggcgtccggccggccgaagcgcatattgctcgcgacggtcccggcgaacagatacggacgctgcggcacatagcccacggcggcggccatcaccgacgggtccagttcgcgcacatcggtgccgccgatccgcacctcgcccgcggtgacgtcgaccagccgcaggatcagatgcagcagcgtggtcttgccggagccggtgctgccgaggaccgcgacccgctcaccgggccgcagcgtcagcgacagatcccgcagcacggcgttctcggcacctgggtagcggaactcggcgcccaccagctcgacctgcccggcgggacccgccatgggccgcggcgaggcgggcgggacgacatcggtctccgcctggaggacctccgtgatccgctccgcgcacacccgggcccgcggcacgttcaggaacacgaaggcgagcatcaccacggacatcaggatgagcgacagatagctgaggaaggcgctgagcgcaccgatctgcatgctgcccgagtcgatccggcgggcccccacccacagcagccccagggtgaagaggttcatcagcagcagcaccgacggcagcatcaccgcgatgagccggcccacccccagggacaggccgaggagttcctcgttggtgcgggtgaagcgctcgccctcatgggcgtcgcgggcgaaggaacgcaccacacgcaccccggtgatgcgctcccgcagcagtcgcccgatacggtccagcgtctgctggagccgggcgtacagcgcggccatccgggacagcagcagaccgaagcagacggccaccaccagcaccatcgggaccaggatcagggccagcgtcacatcctggcgcagcgcgagcagcacactgcccaggcacatcaggggagccgccacgaagatgccgaggccggactgggcgaggttctgcacctgctggacgtcgttcaccgaacgggtcagcagggacggagtgccgaaccggccgatctcacgggcggagaagtccagcacccggcggaagaccgcggaccgcagatcccggccgagagcggtggaggtgcgtgcgcccagcgccgcggcacccagcgccgcggcgatctgcaccagcgccaccacggccatcgtggcgccgatggtgccgatgcggtcgctgtcgtggcggacgactccctcgtcgatgagtgcggcgcccagcgtcggcagcagcagcgtgccgaggatctgcaccagatgcaaaacgaccagagcgacaacggacccggcataggggcgcagttgtgtgcgcatgagtctcaacagcacggaatgacaccccggttgacggcggcccgcagccgcccgttggaacggagtcggccagcgcaccgacggggccattggaccccgcaccgcacccggatttccctagtgttcgccgccgtcgcgtcggccgcccgggaattccctagtaagttcgagaaacgttgacattgacttccgtcgcttgggagcgtgccggtgtctatcgtccggacagctttctcgaagccaggtcgtcgaccgagcgcagcgtttggacggtctcttcgtggtctgagatggggatggaatgcccaagcgcgcactgatcaccggaattaccggccaggacgggtcctatctggcggagcacctgctgtcgcaggggtaccaggtgtggggtctcatccgtggccaggccaacccacgcaagttccgggtgagccggctcgcctccgaactctccttcgtcgacggtgacctcatggaccagggcagcctggtctcggcggtcgacaaggtacagccggacgaggtgtacaacctcggcgcgatctcgttcgtgccgatgtcctggcagcaggccgagctggtcaccgaggtgaacggcatgggcgtgctgcgggtcctggaggccatccgcatggtgagcggactgagcatgtcccgcaccgcgggcaccgagggccagatccgtttctaccaggcgtcctcctcggagatgttcggcaaggtcgccgagacgccgcagcgggagaccacgctcttccacccgcgcagcccctacggcgcggccaaggcgtacggtcacttcatcacccgcaactaccgtgagtccttcggcatgtacgcggtctccgggatgctgttcaaccatgaatcgccccgccgtggccaggagttcgtgacccgcaagatctccctcgcggtcgcccgcatcaagctggggctccaggacaagctggccctcggcaacatggacgcggtccgcgactggggttacgcgggcgactacgtccgcgccatgcatctgatgctccagcaggacgcccccgacgactatgtgatcggcaccggggagatgcacaccgtgcgcgacgcggtccgcttcgccttcgagcacgtgggcctcgactggaaggactacgtcgtcgtcgaccccgacctcgtgcggcccgccgaggtcgaggtgctgtgcgccgacagctccaaggcacaggcccagctgggctggaagccgagcgtcgatttccaggaactcatgcggatgatggtcgacgccgacctcgcgtccgtttcccggcagaacgaactcgacgaccttctgctggcacacagctggtaacgggcgaaagcccctccaagtactagggatttcctgccggacctccggccacactcgcctcatggcttctcctgcctgggagagccatgtggcgagtcggtgttctgcggcatggtgcagagatttcggcgatgcggtacggccgtcggccgtcgggatgggttgagatggacaacgaacagaagctccgggattacctcaagcttgcgacggccgatctgcgacgcgcccggcggcgtgtgggcgagctggagtcggcgtcccaggaaccgatcgccatcgtcggtatgacctgccgctacccaggcggcgtcagcagccccgaggacctgtggcggatggtggaggccggcgagaacggcgtcaccccgttccccaccgaccgcggctgggacctggaggcgctcgcctcctcgccgacctcccggggcggattcctccatgacgcgcccgagttcgacgcggacttcttcggcatctcgccgcgcgaggcggtcgccatggacccgcagcagcgggtcgtcctggagtccgcctgggaggcgttcgagcgcgccggcatcaaccccacctccgtcaagggcagccgtaccggcgtcttcatcggcgccatggcccaggactaccgggtcggtcccgccgacggcgccgagggcttccagctcaccggcaacaccggcagcgtgctgtccggccgtatctcgtacaccttcggcacggtcggtcccgcggtcaccgtcgacaccgcctgttcctcctccctggtcgccgtccacctcgccacccaggcgctgcgcgcgggtgagtgcaccctcgccctcgcgggcggcgtcaccgtcatgtcgggccccggcaccttcatcgagatgggccgccagggcggactgtccgtcgacggccgctgccgctccttcggcgacaccgccgacggcaccggctgggccgagggcgtcggcatcctcgtcctggagcggctgtccgacgcgatccgcaacggccgcgagatcctcgccgtggtccgcggcaccgccgtcaaccaggacggcgcctccaacgggctcaccgcccccaacggcccgtcgcagcaggccgtcatcgagcaggccctcgtcaacgcccggctctccgcgggcgatatcgacgtcgtcgaggcgcacggcaccggcaccaccctcggcgaccccgtcgaggcgcaggcgctgctcgccacctacggtcagcagcgcgacgaggacaagccgctgctgctcggctcggtgaagtccaacatcagccacacccaggccgccgccggtgtcgccggtgtcatcaagatggtcatggcgatgcggcatggggtgctgccccgcaccctcctcgccgacgagcccacccgccacgtcgactggtcccagggcgccgtccgcgtcctcaccgagaacaccgaatggcccgccaccggcgccccccgccgcgcggccgtctcctccttcggcatcagcggcaccaacgcccacaccatcgtcgagcaggcccccgagccggagcccgccgaccccgaggacgacgccccgagcaccccggccgccgtcaccggcgtgctgcccgtgctgctctccggccggagccccgaggtgctgcgcgcccaggccgccgccctgctcaccaccctcggcaccggcaccccgcccgcgccggcggacctcgcctactcgctcgccaccacccgcaccgctttcgagcaccgggccgtgctgctcgcctcggacctgcccgagctgaccgggcggctcaccgcgatcgccgagggcaccgatccggccgtcctggccgacaccgtcaccggcaccgcgagaaccgagacccgcctcgccgtcctgttcaccgggcagggcgcccagcgtctcggcgccggccgtgaactggccgcccgcttcccggtgttcgccgccgccctggacgccgccctcgacgccttcaccccccacctggacgtgccgctgcgcaaggtgctgtggggcgaggacgccgaccgcctggaccgcaccgagtacgcacagcccgcgctgttcgccgtcgaggtcgccctctaccgtctgctggagtccttcgaggtcaagccggaccacctggccggccactcggtgggcgagatcgccgccgcgcatgtcgccggagtgttctccctcgacgacgcggccaccctggtcgccgcccgcggccgtctcatgcaggcgctgcccgagggcggcgccatggtcgccgtgcaggcgagcgaggacgaggtcgccccgctgctcgccggccacgaggacctggtgtccctcgccgccgtcaacggcccctccgccgtcgtcctctccggggacgagaccaccgtcaccgagctggcggcccgcctggccgccgacggacgcaagacgagcaggctgcgtgtctcgcacgccttccactcgccgctgatggccccgatgctcgacgagttccggaacgtcgtcgagggcctcaccctgcacagcccgctgctgcccgtcgtctccgacgtcaccggagagccggccaccgtcgcccagctcacctcccccgactactgggtcgaccatgtccgtcaggccgtccgcttcgccgacggcatcgactggctcgcccggcacgatgtcaccgccttcctcgaactcggccccgacagcgtgctgtccgccatggcgcagaactgcctggacgccgcaggatccgacgccctcaccgtgcccgcactgcgcgagggccgccccgaggaccacaccttcaccgccgcgctcgccgcgctgcacacccagggcaccgcgctgcactgggacgcctgcttcaccggcaccggagcccgccgcaccgacctgccgacgtacaccttccagcgccgccgctactggccgcgcgccgtccagggcggcgccgccgacctgcgctccgtcggactcggcgccgcccaccacccgctgctgtccgccgcggtctccctcgccgactccgagggcgccctgctgaccggccggatctccctgctcagccacccgtggctcgccgaccacaccgtccgcggcgccaccctgctgcccggcaccgccttcctggagctggccgtccgcgccggcgacgaggtcggctgcgaccgcgtcgacgaactgaccctggccgcaccgctcgtcctgcccgagcagggcggcgtccaggtgcagctgtggatcggcaacccggacgcgtccggccgccgcagcgtcaccgtctacggccggcccgacgccgacgaggacgccccctggacgtcccacgccaccggtgtgctctccgcctcccgcaccaccagcgacttcgacgccaccgtctggcccccggccgacgccgagaccctccccgtggacggcctctacgagcggctcgccgagggcggcttcggctacggcccgctcttccagggcctgcgggccgcctggcgccgcggcgacgaggtgttcgccgaggtcgtgctgcccgagtccggccacaccgacgccgagtccttcggactgcaccccgcgctcctcgactccgccctgcacgcggcgtcgttcgtcgacctcgacgagcgcgccgccggcggactgcccttctcctgggagggcgtcagcctgcacgcgagcggtgccaccaccctgcgggtgcggctggccccggccgccggtgacgccgtcgcgatcgccgtcgccgacgactccggacagctcgtcctgtccgccgactcgctgatcctgcgcgccgtcgcggcccgcgagatcgacgccgccgccgcgctggtccgcgacgcgctgttccgcctcgactgggtgcccgtcacggccgtcgccgcctccggcaccgccgcggcactggtcggcgaggaccccttcggcctgcgcgccctgccccagttcggcgacctcgccgtccacccggatctcgcggatctggccgccgccgacggcgctgtcccggacaccgtgctgctgccgctcaccggcaccggacccgacgccgacccggtcaccgccgcacaccgggccgccaccgaggccctcgccgccgtccggacctggctcgagcaggacgagcggttcgccgcctcccggctcgccctggtcacccggggcgcgaccaccggccacgacccggccgccgccgcggtctggggcctggtgcgctccgcccagtccgagaaccccggccgcttcctcctcgtcgacctcgacgccgaccaggacacccccgcgctcccggccgccgcgctcacctccgaggagccgcagctcgccgtgcgcggcgaggagctccgcgccgcccgtctggtccgccgtcccgcttccaccgccgaggccgttcccgccttcggtggggagggtgcggtgttggtgacgggtggtaccggtggtctgggtgcggtgctggcgcgtcatctggtggccgagcacggtgtgcgtgagctggttctggtcagtcgtcgtggtggtgcggcggcgggtgcggccgagctggtcgcggagctggcggagtcgggtgcgcgggcgacggtggtggcctgtgatgtgacggaccgtgcggcggtggccgagctggtggccgcgcatccggtgtcggcggtggtgcactccgccggtgtcctggacgacggcatggtcggcacgctgacccccgaacgcctcaccaccgtgctccgtcccaaggtggacgcggcctggaacctccatgaggccacccgcgacctggacctgaaggcctttgtgctgttctcgtcggtcgccggcgtcctgggcagccccggtcaggccaactacgccgcaggcaacgccttcctggacgccctcgcggcccaccgccgggccgcgggcctgccgggtctgtcgctggcctggggcccctgggagcagaccggcggcatgaccggcggcatctccgaggacgacctgcgccgcatggcccgcgccggaactccggccctcaccgtcgagcagggcctcgccctcctcgacgccgccctcgacggcgacgacgccgcgctcgcccccgtccgcctggacctctccgtcctgcgggcccagggcgaagtgccgccgctgctgcgctccctgatccgcggccgctcccgccgtgccgccgtcgccgggtccgccaccgccggcgggctcgcccagcgtctggcccgcctggacgccgagagccgcgacgaactggtcctggacctcgtccgcggccaggtcgccctcgtcctcggtcacgccaccggcgccgagatcgacgccggccgcgccttccgtgagctgggcttcgactccctcaccgccgtcgagctgcgcaaccgcctgaacacggtcaccggactgcgcctgcccgccaccctggtcttcgactacccgacggtcagccacctcgcctcctatgtcctggacgagcttctcggcaccgaggtcgaggcggaggtcgtgcagcgcggcacggcggccgtggcggacgatccgatcgtgatcgtgggcatggcctgccgctatccgggcggggtcacctcgccggaggacctgtggcgcctggtcaccgagggcacggacgccgtctccggcttccccgtcaaccgtggctgggacgtggagaacctctaccacccggaccccgaccaccccggcaccgcctacacgcgttccggtggtttcctgcacgaggcgggggagttcgaccccggcttcttcggtatgagcccccgtgaggcgctggcgacggactcgcagcagcgtctgctcctcgaggcgtcctgggaggcgatcgagcgggccgggatcgatccggtcggtctgcggggcagtgcgaccggtgtgttcgccggtgtgatgtacagcgactacagcgcgatgctgggaagccccgagttcgagggcttccagggcagcggcagctctccgagtctcgcctcgggccgcgtctcctacacgctgggcctggagggtccggcggtcacggtggacaccgcgtgctcctcgtcgctggtggcgatgcactgggcgatgcaggccctgcgctcgggtgagatctcgctcgccctggccggcggtgtgaccgtgatgtccacgcccgccgtgttcgtggacttcgcgcggcagcggggtctgtcgcccgacggccgctgcaaggcgttctcggactcggccgatggtgtgggctggtccgagggtgtcggcatgctggtgctggagcggcagtccgacgcgatccgcaacggccaccagatcctcgcggtcgtccgcggttcggccgtcaaccaggacggcgcctccaacggcctcaccgcccccaacggcccgtcccagcagcgcgtcatccgccaggccctcgccagtggtgggctctcggcgggcgacgtggatgtcgtcgaggcgcacggtacgggtaccaccctgggtgacccgatcgaggcgcaggcgctcctggcgacctacggccgggaccgtgacccggagcagccgctgctgctgggttcggtgaagtccaacatcggtcacacgcaggccgcggccggtgtcgcgggtgtgatcaagatggtcatgtcgatgcgccacggtgtactgccccgcaccctgcacgtggacgccccgtcctcgcacgtcgactggaccgaaggcgcggtcgagctgctcaccgagcagaccgcctggcccgagaccggccgtccgcgtcgcgccgccgtgtcctccttcggcatcagcggcaccaatgtccacaccgtcctggagcaggcgccgggcaccaccgtgcccgccccggccgcccccgagcgcaccgccggcgccgtcccgctgctgctgtccggccgcacccgcgacgccctgcgcgcccaggccgcccgcctgctcacccacctccagaaccaccccgagccgtccctcgccgacctcggccactccctggccaccacccgctcccgcttcgagcgccgcgccgcggtgatcgcgcaggaccgcgaaggactgctcgcctccctcggctccctcgccgccggccgccccgacccggccgtcgtcgagggtgaggccgccgggcgtgcccgggtcgccgtgatgttctccggccagggcagccagcgcgccgccatgggccgcgaactgtacgagacccagccccggttcgccgccgcgttcgacgaggtgtgcgccgccctcgacccgctgctcgaccggccgctgcgcgaggtcgtgttcgccgccgagggcagcgaagaggccgcgctgctcgaccgcaccggctggacccagcccgcgctgttcgccgtcgaggtcgccctctaccgacttgtcgagagctggggcgtccgcgccgacttcgtcaccggtcactccatcggtgagatcgccgccgcgcacatcgccggggtgttcaccctccaggacgccgcccgactggtcgccgcccgcgccaccctcatggaggcgctgccgtccggcggcgccatggtcgccgtgcaggccaccgaggaagaggtcgccccgctgctcggcgagggcctgtccgtcgccgccgtcaacggccccacctccgtggtcgtctccggcgacgaggaccccgccgtggagctcgccgccgagttctccggccgagggcgccgcaccaagcgtctacgggtgagccatgccttccactcgccgcacatggacgccatgctcgacgcgttccgcacggtcgccgagacgctgtcctacgccgccccccgcatcccgctggtctccgacctgacgggccgcagggccgacgacgccgaggtgcgcaccgccgactactgggtgcgccacgtccgtgaggccgtacggttcgccgactgcgtccgcaccctgcgcgacgccggcgccaccctcttcctcgaactcggccccgacggactgctgaccgccatggccgaggacaccctcggcgacgagcgttacgaccacaacacggcactcgtcccgctgctgcgcgccgaccgccccgaggagagcgccgccgccaccgccgcggcccgcctccagatccacggcgtcgacctcgactggaccgcgtacctggccggcaccggcgcccgccgcgtcgacctgccgacctacgccttccagcacgcccactactggccgcagctgccctccgccgcgccctccccggccggcgacccggccgaccagaagctgtgggccgccgtcgagcgcggcgacgccgccgaactcgccgccgtcctcggcctcgacgaggacagcctcaccccgctggactccctgctgcccgcgctgagctcctggcgccgcggcaaccaggagaaggcgctcctcgacacgctgcgctaccgcgtcgagtggacccggctgagcaagcccgccgcccccgtcctcgacggcacctggctgctggtctcctccgacgccaccgccgacgacgagaccgagctgctcgacggcctggccgaggcgctcggcgcgcacggcgcccgggtccgccgcctcgtcctggacgccgactgcgccgaccgggccgtgctcggcgcccgcctcgccgacaccgagaacgccgacaacaccgcccaggtgctgtccgtgctgccgctcgacgagcgccccaccgacggccccgccggcttcacccagggcctcgccctgaccatcgccctcgtccaggccctcgccgacaccggcgcccacggccggctgtggaccgccacccgcggcgccgtctccaccggacccgccgacccggtcacccaccccgcccaggccaccgcctggggcatgggccgcggtgtcgccctggagcacccccgcctgtggggcggcctcgtcgacctcccggccgacttcgaccgcggcgccggacagcgcctcgccgaggtcctcgcggtcaaggacgcccccgacggcgaggaccaggtcgccctgcgcgccaccggtgtgcacggccgccgcctggtccgccacatcgtcgacgagctgccctccgccgaccagttcaccgcctccggaagcgtcctgatcaccggcggcaccggcggcctcggcgccgagaccgcccgctggctggcccgctccggcgccgcccacctcgtcctcaccagccgccgcggccccgacgcccccggcgccgccgaactccgcgccgaactggagcagtcgggcgcaagcgtctcgatcgtggcctgcgacgtcgccgaccgtgacgccctcgccgccgtcctcgacggactgtccgccgaccagcccctcaccggtgtggtgcacaccgcgggcgtcggccactacggcccgctggacgcgctgacgcccgccgagttcgccggcctcaccgccgccaagctggccggcgccgcccacctcgacaacctcctcggcgaccgcgaactggacttcttcatcctgttcggctccatcgccggcgtctggggcagcggcgaccagagcgcctacggcgctgccaacgcctacctcgacgccctcgccctggcccgccgcgcccgcggcctcgccgcgacctccatcgcctggggtccctggggcggcaccggcatggccgccgacgacgccgtctccggcaccctgcgacgccagggcctcggcctgctcgaccccgcgcccgccctcaccgagatgcgccgcgccgtggtccgccaggacgtcaccgtcaccgtcgcggacgtcgactggacccgctacgccccgctgttcacctccgcccggcccagcgccctcatcagcgacctgcccgaggtccgtgccctggccgccgagaacacccccgccgacaccggcgacgcctccgagatcgtccagcgggtccgctcgctgtcggagccggagcaactgcgcctgctcaccgacctggtgcgcaccgaggccgcgaccgtgctcggtcactcctccgccggcgccgtaccggaggaccgtgccttccgcgagatcggcttcgactcgctgaccgccgtcgaactgcgcaagcacctcggcgccgccaccggcctctcgctgccgagcaccatggtgttcgactacccgaccccgctggagctggcccagtatctgcgggccgagatggtcggctccgtcctggaggtcgcgggccccgtcgccaccggcggtaccgacgacgaacccatcgccatcatcggcatgagctgccgctaccccggcggagtcagctccccggagcagctgtgggacctggtgctctccggcaccgacgcgatcaccgacttcccggtcaaccgcggctggaacaccgccgggctctacgaccccgaccccgaccaccctggcaccacctactccacgcagggcggcttcctgcacgaggccgacgagttcgaccccatgttcttcggcatctcgccccgcgaggcgctggtcatggacccgcagcagcggctcctcctggagaccacctgggaggccttcgaacgcgccggactcacccccgacaccctccgcggcagcctgaccggcaccttcatcggctccagctaccaggagtacgggatgggcgccggcgacggcgccgagggccacctggtcaccggcaccagccccagcgtgctctccggccgcctcgcctatgtcttcggcctggagggcccggcggtcaccgtcgacaccgcctgctcgtcctcgctggtcgccctgcacctggcctgccaggcgctgcgcaacggcgagagcaacctcgcggtcgcgggcggcgccaccgtcatgaccacccccaacgcgttcgtggcgttcagccggcagcgcgccctcgcgcaggacggccggtgcaaggcgttctccgagagcgccgacggcatgaccctcgccgaaggtgtcggcatcgtcctcgtcgagcggctgtccgacgcccgccgcaacggccacccggtgctcgccgtgatccgcggctccgccatcaaccaggacggcgcctccaacggcctcagcgcccccaacggcccctcccagcagcgcgtcatccgccaggccctggccaacgcccgggtcgcccccggcgagatcgacctcctggaggcccacggcaccggcaccccgctcggcgaccccatcgaggcgcaggccctgttcgccacctacggccgcacccgtacccccgagaccgcgctgctgctcggctcggtgaagtcgaacatcgggcactcccagtccgccgccggtgtcgcgagcatcatcaagatggtcatggcgctgcgccacggtgtgatgccccagaccctgcacgccgacgagccctccagccacgtcgactggagcccgggcaccgtacggctgctcggcgagaacaccgactggccgcagacgggccgtccgcgccgggccgcggtgtcctccttcggcatcagcggtaccaacgcccacgtcatcctggagcaggagaccgaggcgcccgccgccgaggacgagcagctcgccccggcgcccctgccggtcgccgccggtgtggtgccgtggctgctgtccgcccgtggcgccgccgcgctgcgcgagcaggccgaccgcctcctcacccatctcgtgaccgccgacccggcggcccgcccgatcgacatcggtctgtccctggccacctcccgcgcgctcttcgagcaccgcgccgtggtcgtgccgcccgccggcaccgacccgctggaggcgctgcgcgccgtcgccgcagacgggccctcgggcgtcgtcgcccgcggtgtcgcggacgtcgcgggccgcaccgtcttcgtcttccccggccagggctcccagtgggcgggcatgggcgcccagctcctggacgagtccccggtgttcgccgagcggatcgccgaatgcgccgccgccctcgccgagttcaccgactggaacctcatcgacgtcctgcggggcgccgagggcgccccgacgctggagcgcgtcgacgtcgtccagcccgcctcgttcgccgtgatggtctccctggccgccgtgtggcgggcccagggtgtcgaacccgacgcggtggtcggccactcgcagggcgagatcgccgccgcggtggtctccggcgccctgtcgctgcgcgacggcgcccgggtcgtgacgctccgcgcccaggccatcggccgcagcctcgccgggcgcggcggcatgatgtcggtggcgctgcccgtcgccgaggtggaggcccgtctggaggcgttcgagggacgggtgtccgtcgccgccgagaacggcccccgctcctccgtcgtcgcgggcgagcccgaggccctggacgaactgcacgcacagctcaccgccgaggagatccgggcccgccgggtcgccgtggactacgcctcgcactccccccatgtcgaggacctgcacgacgagatcctcgaactgctggccgaggtcgcgccgcggacgtccgagatcccgttcttctcgaccgtcaccggcgactggctcgacaccaccgtcatggacgccggctactggtaccgcagcctgcgcggccgggtgctgttcgcggacgcagtccgtgacctgatcgccgccgaccaccgtgccttcatcgaggtcagctcccaccccgtgctcgccatgtccgtccaggacatgatcgacgacgccggagtggcgggtgtcgcctccggcaccctgcgccgcgacaacggcggtctcgaccgcttcctgctgtccgccgccgaggtgttcgtccgcggtgtccaggtcgactgggccgccgtgttcgaggggaccggtgcgtcccgggtcgacctgcccacctatgccttccagcacgagaacctgtgggccatggccgccgccccggaggccgtcaccgcggccgaccccgaggacgccgcgttctggaccgccgtcgaggacggggacgtctccgccctgaccgcggccctcggcaccgacgaggactccgtcgccgccgtgctgcccgcgctgtcctcctggcgccgggcccgcaaggagcgctccaccgtcgactcctggcgctaccgcccgacctggaagcccgtcaccaagctcccgcagcgcaccctggacggcacctggctgctggtgtccgccgacggcgtcgacgacaccgacgtcgccgaggcgctggagacgggcggcgccgaggtgcgccggctcgtcctggacgagagctgcaccgaccgcgccgtgctgcgcgagcgcctcaccgacgccgacggcctgaccggcatcgtctccgtcctcgcgggcgccgagcggaccggtgccgtccccggcaccgggctcgtcctcggggtcgccctcaccgtcgccctcgtccaggcgctcggcgacgccggtatcgacaccccgctgtgggccctgacccgcggcgccgtctccaccggccgctccgacaaggtcaccgcgcccgtccaggcccaggtcaccggcatcggctggaccgccgcgctggagtgcccgggacgctggggcggtgtcgtcgacctgcccgagacactggacgcccgcgcgggccagcggctcgcggccgtcctcgccggcgcgctcggcgacgacgaccagatcgccctgcgctcctccggcgtcttcacccgccgtatcgtccgcgccgacgccgcccccgacggctccgcccgcgactggaagccgcgcggcaccaccctggtcaccggcggctccggcaccctcgccccgcacctggcgcgctggctcgccgagcagggcgccgaacacctggtcctggtcagccggcgcggccccgaggcgcccggcgccgccgaactccgcgccgagctggcggaacgcggcaccgagacgaccctcgccgcctgcgacatcaccgaccgtgacgccgtcgccgccctcctcgaaagcctgaaggccgagggccgcaccgtccgcacggtggtgcacaccgcggccaccatcgaactgcacaccctcgacgccaccaccctcgacgacttcgaccgcgtcctcgccgccaaggtcaccggcgcgcagatcctcgacgaactcctcgacgacgaggagctggacgacttcgtcctgtactcctccaccgccggcatgtggggcagcggcgcgcacgccgcgtatgtcgcgggcaacgcctatctggcggccctcgccgagcaccggcgcgcccgcggcctgaccgccctgtcgctgtcctggggcatctgggccgacgacctccagctcggccgagtcgacccgcagatgatccgccgcagcggcctggagttcatggacccgcagctcgcgctgagcggcctcaagcgggccctcgacgacgacgagcaggtcatcgcggtcgccgacgtcgactgggagacgtaccaccccgtctacacctcggcccggcccaccccgctgttcgacgaggtccccgaggtgcagcggctcaccgccgctgccgagcagagcgccggggaccccgcccggggcgagttcgccgccgcgctgctggccctgcccgccgccgaacagcaccgcaagctgctggagaccgtccgcaccgaggccgcctccgtgctcggcctgtcctccgcggaggacctcaccgaccagcgcgccttccgcgacgtcggcttcgactcgctgaccgccgtcgggctgcgcaaccggctcgcgtccgtcaccgggctgacgctgccgtccacgatggtcttcgactacccgaacccggccgccctggccggcttcctccactccgaactggccgacgtccactccgcgggcgccgtcgccgtcaccgccggcgcgcccgtcgacgacgacccgatcgccatcgtcggcatgagctgccgctaccccggcggcatcacctccgccgagcagctgtggcgggtctcgctggaggaggtcgacgcggtctccgtcttcccggccgaccgcggctgggacgccgaggccctctacgaccccgacccggacgcctccggccgcacctactcggtccagggcggcttcctgcgcgacgtcgccgacttcgaccccggcttcttcggcatctcgccccgcgaggccctgtcgatggacccgcagcagcgcctgctcctcgagacggcctgggaggtcttcgagaacgccggcctcgacccggtcgcccagcgcggcagccgcaccggcaccttcatcggcgccagctaccaggactacggcgccgccgtgcccggcagcgagggctccgagggccatatgatcaccggctcgctgcccagcgtgctgtccggccgagtctcctacctcttcggcctcgagggcccggccgtcaccctcgacaccgcctgctcgtcctcgctggtcgcgatccacctggcctgccagtcgctgcgcaacggcgagagcaccctcgccctcgccggcggtgccagcatcatgtccacccccatgtcgttcatcggcttcagccggcagcgggccctcgccgaggacggccgctgcaaggcgtacgccgagggcgccgacggcatgaccctcgccgagggcgtcggcctgatcctgctggagcggctctccgacgcccgccgcaacggccacgaggtcctcgccgtcatccgcggctccgccgtcaaccaggacggcgcctccaacggactgaccgcccccaacggcccctcccagcagcgcgtcatccgccaggccctcgccaacgcgggcgtcgaggcgaacgacatcgacgtcctggagggccacggcaccggcaccgccctcggcgacccgatcgaggcgcaggccctgttcgccacctacggcaaggaccgcgaccccgagcggcccgtgctgctcggctcggtgaagtccaacatcggccacactcagatggcctccggcgtcgccagcatcatcaagctggtccacgccctgcgggagggcgtcgcccccaagtcgctccacatcgaccagccgtccacccatgtcgactggtcctccggcaccatccagctgctcaccgagcgcaccgagtggcccgagaccggccgtccgcgccgcgccgccgtgtcgtccttcggactcagcggcaccaatgtccacaccgtcctggagcaggcccccgccgccgacgccccggccgccgaggacacccccgcaccgcgggacgccttggtgcccgtcctcgtctccggacgcggcgaggcggccctgcgcgcccaggccggagccctgctcgacctgctggccgagcgccccggcatccaccccaccgatctggcgttctccctcgccacctcgcgcgccgccctcgagcaccgtgccgcggtcgtcgccgacgaccacgaggcactggtgcgcggtctgaccgcactgcgcgacggactgcccggcgccggactcgtccagggccgcaccggccgcggacgcaccgccttcctgttcaccggacagggcagccagcgccttggcatgggccgcgaactctacgaacgccacccggtgttcgccgacgccctggacgcggtgctcgcccggatcgacggcaccaccgaacgccccctgcgcgacgtgctgttcgccgccgagggctcccaggacgccgccctgctccaccgcaccggctacgcacagcccgcgctgttcgccctcgaagtggcgctgttccggctgctggagtcgtggggcgtgacccccgactacctggccgggcactccgtcggcgagatcgccgccgcgcatgtcgccggcgtcctcgacctcgacgacgcgtgcaccctggtggccgcccgcggccggctgatgcaggcactgcccgagggcggcgcgatggtcgccctggaggccgccgaggacgaggtgctgccgcacctggagggcctcgccgaccaggtctccgtggccgccgtcaacgggccccggtccgtcgtcgtcgccggcgaggaggagcccgtgctcgccctcgccgcgcacttcgccgaacaggggcgccgcaccaagcgtctgcgggtgagccatgccttccactcgccgctcatggacccgatgctggacgacttcgccgccgtggcccgtgctctcacgtaccacgcgccgtcgatcccgttcgtctccaatgtgaccggcaccctcgccgcccccgagcaggtgtgcaccgccgactactgggtctcccacgtccgctccgccgtgcgcttcgccgacggcatcggctggctgtccacccagggcggcgtccagaccttcctggagctcggccccgacggtgtgctcagcggcatggcccgcgagagcctcaccgacgcctcccgcacggcgctgctgcccaccctgcgcggcgaccgcccggaggagcaggccctggtcaccgccgtcgccgcggcccacgcccatggcttcgacgtcgactggaccgcctggttccagggcagcggcgcccgccgggtggccctgcccacctacgccttccagcgcgagcggtactggcccgacaccaccgcggccggcatcaccgcccccgcccccggctccgcactggacgccgagttctgggccgcggtggaacacgccgacgtcgcctcgctcaccgcctccctcggcctggacgacgcgaccgtcaccgcgatggtcccggcgctcaccgcctggcggcagcgccgcggcgaacagtccgcgctcgactcctggcgctaccgcgtcacctggaagccgcgcggcggcgcccccggcgccgcacccaccggacgctggctcgtcctggtcccggccgagcaccgcgacgaggccaccgccgcctgggccgccgatgtcgaggccgccctcgccaccgccaccgtccgcgtggaggtcaccggcaccgaccgcgccgccctcgccgcccggctcaccgaggccgccgacggcgacaccttccagggtgtgctgtcgctgctggcgctggcgcccggtgacgccggacaccccggcgccccggccgcgctcaccctcaccgccaccgccctccaggcgctcggcgacgcccgtatcgacgccccgctgtggaacatcacccgcggagccgtcgccgtcggccgctccgagcaggtcacggcacccgaacaggccgccgtatggggcctgttccgcgccgccgccctcgaactgcccgcccgcgtcggcggcagcgtcgacctcccggaggacctcgacacccaggccgcccgacggctgcgcgggatcctggccgccgccgacggcgaggacgccgtcgccgtccgcgccagcggcgtcttcctgcgccggctcgcccacagccccgccgccgacaccgtcggctccgccttcgacccggcggccggcaccgtcctgatcaccggcggcaccggcggcatcggcggacacctcgcccgccgcctcgcccgcgacggcgccgcccatctgctgctcaccagccgccgcggccccgacgcccccggcgccggcgaactccgcgccgaactggaggagtcgggcgcccgtgtcacgatcgccgcctgcgacgccgccgaccgcgacgcgctcgccgcactcctcgccacggtgcccgaggacgcgcccctcaccgccgtcttccacaccgcgggcgtcgtcgacgaccatgtggtggacgagctgaccccggagagcttcgccaccgtcctgcatgccaagacggtcgccgcccgccatctgcacgacctcaccgccggccacgacctcgccgcgttcgtcctgttctcctcgaccgccggcctgatcggcgccgccggacagggcaactacgccgccgccaactcccagctcgacgccctcgccgagcagcgccgcgcccagggcctgcccgccctgtccgtggcctggggaccctgggccggctccggcatggtcgccgacgccgccgagatcgccgcccgggtgcgccgcggcggattcgaaccgctcgaccccgcctccggtgtcgccgcgctgctgcgcgccgtcgacaacggcgacacctccgtcgccgtcgccgacatcgactgggaacgcttcctgcgcgccttcgcgtcggcccgcccgctgccgctcgtcgccgacctcccggagacggcgggcgccaacgccccggcggcggccgccggcaccggctcaggcctgcgcgagcagctcgccggcctgcccgccgaggagcgccacgcacatgtcctcgatctgctgcgtacccaggtggcggccgtcctcggccactccgacgcccgcaccgtcgacgacgacctggccttccgggacctcggcttcgactcgctcaccgtcctggagctgcgcaacgcgctcaacgccctcaccggcctgagcctgcccgcctccctcgtctacgaccacccgacgccgcgcgagatggcggacttcctgctcgccgaactcctcggctcgctcccggagtccacccgccacgcggtcgccacccgggccgtcgacgaggacccgatcgcggtcgtcggcctcgcctgccgcttccccggcggcgtgagcacgcccgaggagctgtggcagctgctggcggagggccgcgacggcatcaccggcttccccgacgaccgcaactgggacatcgcggccctcggcgccggcgcctccgacaccctgcacggcggattcctcgcccaggtcgccgacttcgacgcccgcttcttcggcatctcgccccgcgaggcgctcgccatggacccgcagcagcgactgctgctcgagaccacctgggaggccctggagcgggccggcatcgacccggccgcgctgcgcggctccaccaccggtgtgttcgtcggcaccaacggccaggactacccgaacctgctgcgccgctcgacctcggacgtctccggctatgtcgccaccggcaacaccgccagcgtgatgtccggccgcctctcctacgccctcggcctggagggccccgcagtcaccatcgacaccgcctgctcctcctcgctggtcgccctgcactgggcgggccacgcgctgagcgccggcgagtgcgacctcgtcgtcgcgggcggcgtctcggtgatgtcgacccccgactccttcgtcgagttcagcacccagggcggcctggcccccgacggccactgcaagccgttctccgacgacgccgacggcaccgcctggtccgagggcgtcggcatcctggtcctggagcggctctccgacgcgatccgcaacggccacgaggtccgcggcatcatccgcggcaccgccgtcaaccaggacggcgcctccaacggactgaccgcccccaacggcccgtcccagcagcgcgtcatccgccaggccctcgcggacgcccgtctcgcgccgtccgagatcgacgcggtcgaggcgcacggcaccggcaccaccctcggcgacccgatcgaggcgcaggccctcctcaacacctacggcccgggccgggacaccgaccagccgctgctgctcggcaccgtcaagtccaacatcggccacacccaggccgccgcgggcgtcgccggcgtcatcaagatgctgctggcgatgcagcacggcaccctgccgcgcaccctgcacgtcaccagccccaccagccatgtcgactggagcagcggcgcggtgtcgctgctcaccgaggagcgcgactggcccgagaccggacggccgaggcgcgccggtgtctccgccttcggcgtcagcggcaccaatgcgcatgtcatcgtggagcaggctcccatcgacgagcccgtggtcgagcccaccgaggagcccgcccccgccgccgaggtcaccaccgtcccgtggatcgtctccggccgcagccgcgaggcgctccaggaccaggtcgaccggctcaccgcgtacgccgccgcccgcccggagctctccccgctggacgtcggccgctccctggccaccgaccgcaccctcttcccgcaccgcgccgtgctgctggccggccccgacggggtgcgcgaggcggctcgttcggtcgcgtcccgcacccggggccgtaccgccttcctgttctccggccagggcgcccagcgcgccctgatgggccgcgaactctacgagcggtacccggcgttcgccgacgcgctggacacggtcctcgcgcagttcgacaccgcgctggacttctcgctgcgggaggtcctgttcgccgagccgggcacgcccgaggccgagcggctgaacgagaccggctggacccagcccgcgctgttcgccgtcgaggtcgccctccaccgcctcgtggagtcgtggggcctgacccccgaccaggtcgccggtcactccatcggtgagatcgccgccgcgcatgtcgccggggtgttctcgctggaggacgcgtgcgcgctggtcgccgcccgcgcctccctgatgcaggagctgccgcggggcggtgcgatgctcgcggtgcgcgccaccgaggaggaggtcgccccgcatctgacggagcgcgtctccgtcgcggcggtcaacggtccttcggcggtggtcgtcgccggtgacgaggccgaagtcgccgcgatcgccgcgcacttcgaggcgcaggaccgcaagaccacccggctgcgggtgagccatgccttccactcgccgctcatggacccgatgctcaccgccttcgcccgggtcgcggagtccctcacctaccacgagcccgtcatcccgctggtctccacggtgaccggcgtacccgccggagccgaactcctcacccccggctactgggtgcggcacgtccgcgagaccgtacggttcgccgacggcgtccgggcgctgcgcgaggacggtgtcaccaccttcgtcgagatcggtccggacggacagctcaccgccgccgcccagcagaccctggacgcgtccggcgacgaggcacctgccaccgtcgtcgcgctccagcgccgcgaccgctccgaggagaccaccctcctggagggcctggccaccctgcacacccagggcgccggacccgactggaccgcctggttcgccggcaccggcggacaccgcgtcgaactgccgacgtacgccttccagcgcgagcgctactggcccgagcccgccgcgcccggcgccaccggcgtccaggaccccgtcgacgccgccttctgggccgccgtcgagcgggaggacctggagagcctcacggcctccctgggcctggacggcgacaccgtcaccacgatggtgcccgcgctgtccgcctggcgccgccgcaccgacgcccgctccaccgcccgcgactggcgctaccacgagagctggaccgcgctcggcaccccggcgcactccgcgggagccggccgcgtcctggccctggtgcccgccgaacacgccggcacggactgggcggagaccctggtcgccgccctgggcgccgacccgctcgtcgtggacgggacctccggctccctcgcccgggaactcgcggacctagtgccccaacaggcgacgttcgccccgtcgcgacgcccggcacgcactctcgccgcaccggccgaaagcccgagtacgtccagtacggggacttccggccggcacaccgagagcacgcaccggacgccgctcctggacgggcaaacgtcacctgccgggacactggacccggccggctggaccgtcgtggtctcgctgctcgccgcgggcaccggcaccctgccggcccccgacgccgcaccggccgccgtcctggaggccctcgaggccgccggcgtcgacgccccgctgtggtgcgtcacccgcggcgccgtctccgtcgccggggaggcacccgccgccgtcggccaggccgccctctggggcatgggccgggtcgccgcccttgagcaccccgagcgcttcggcggcctcgccgacctcgcgcccgacgccgacgccgccaccgctgccctgctcgccgcccacctggccgagcccggcggcgaggaccagatcgccgtccgcgccaccggtctcttcggccgccgcctggtgcgcaccgccgtcgcccccggcgactccggatggcgcccgcacggcaccgtcctggtcgtcggcggcacccgcgccatgggcgcccgcgccgcccgctggctcgcccgcgagggcgccgcccgcctggtgctgaccacctccccggccgacagtgccaccgacaccgaggagctgagggccgaactcggccggctcggcgccgaggtcaccgtcgccccgtacgacggcggcgaccgggacgcggcccgcgccctcctggacggactgaccggcctcaccgcggtcgtgtacgccgacgacaccccggcggacggcccggccgccgccctcgccccggtggacaccctcgccgaggccgccaccggccgctccctggacgcgttcgtgctcttcggctccgtcgccggggtctggggcgtgcgcggccacaccgacgaggccgcggaaggcgcctatgtggacgccctcgcccgcgccctgcgcgccgaggggacgcccgcgctggccgtctcctggaacgcctgggccgagctgaccgacccgtcgaccacccgccatctgcggatgaacggcctgccggtgatggacgccgacgccgccctgaccgccctcgccggtgccgtcgccgacggctccgccgcggtcaccgtcgccgacgtccgctgggacaccttcgccccggcgcaccacgagacccggccgaccgcactcttcgacggcctgcccgaggccaggaccgccctcgccggcgcggcccgcgaccgtgccgagggggccaccgcctccgacggctacggccggtggctcctggagcagcccgccgccgaccgggacgacatcctgctcgccctggtcagcgagaaggccgccttggtcctcggccacgcggacaccacgctcgtcgaacccgacctgcccttccgggacctcggcttcgactcgctgaccgccgtggacctgcgcaaccagctgaccgccgccaccggcatcacactgcccgcgaccctggtcttcgaccaccccaaccccgcggcgctcgccgcacacctgcgcaccgaactcctcggcggcgccacggacaccgccgccccggtggccgcgcccgccgccaccggcgacgacccgatcgtcatcgtcggcatggcctgccgcttccccggaggcgtcaactcgcccgaggacctctggcagctcgtcctcgacgaggtggacgccgtcggcgacttcccggccgaccgcggctgggacctcgacgccctggccggcgacggaccgggccgcagcgccaccgaccagggcggattcctctacgacgccaccgacttcgaccccggtctgttcggtgtctcgccgcgcgaggccatggtgatggacccgcagcagcggatcctgctcgaggcgtcctgggaggccctggagcgggccggcatcgaccccggcaccctgcgcggcagcggcaccaccggcgtcttcgtcggcggcggctccggcgactaccggccgccggccgagtacggccagtggcagaccgcccagtccgccagcctgctgtccggccgcctcgcctacaccttcggcatccagggccccacggtgtccgtcgacaccgcctgctcctcctccctggtcgccctgcacctggccgcccaggccctgcgctccggcgagtgctcgatcgccctggccggcggtgtcaccgtgatggccaccccggtcggcttcgtcgagttcagcgcccagggcgcgctgtcgcccgacggccgctgccgcgccttctccgaggacgccaacggcaccggctggtcggagggtgtcggcatgctcgtcgtcgagcgcctgtccgacgcccgccgcaacggccaccgggtgctcgccgtgctgcgcggctccgccatcaaccaggacggcgcctccaacggactgaccgcgcccagcggccccgcccagcagcgcgtcatccgccaggccctcgccaacgcccgcctggaggccgccgacgtcgacgccgtcgaggcgcacggcaccggcaccaagctcggcgacccgatcgaggcgcaggccctgctcgccacctacgggcaggaccgcgagcggcccgtgctcgtcggctcgctgaaggccaacatcggccacacgcaggccgcgtccggtgtcggcggcgtcatcaagatggtgctcgccatgcagcacggtctgctgccgcgctcgctgtacaccgagaacccctcctcgcacgtcgactggaccgcgggcaacgcccgcctgctcaccgagctgaccccctggcccgagtcggagcgggtgcgccgggccgccgtctcctccttcggggccagcggcaccaacgcccacctgatcctggagcagccggagcccgtcaggcagcccgtggagggtgcccggcccgccgaggacggctcgcccgtcccggtcctgctgtccggccgctcgcagaacgcgctgcgcgcccaggcccgcaagcttctggcccatgtggcgggacaccccgaggagacggtcaccgacctggcccgcaccctggccaccacccgcgccgtcttcgagcaccgggccgcggtgaccgccgccgaccgggacgccctgatcgccggactcaccgccgtcgcggagggcaccaccgccgcgcacatcgtcgagcaccaggtggagcgcaccggcaagcacgccgtgctcttctccggacagggctcccagcagctcggcatgggccgggacctgtacgagcggttcccggcgttcgccgaggcgttcgacgcggtcctcgcccggctcgacggccacctcggcgcctcgctgcgggacgtcgtctggcacggcgaccaggagaccctgaacgagaccgggcacacccagcccgcgctgttcgccttcgaggtcgccctgtaccgcctggtggagtcctggggagtgacgccggacttcgtcgcgggacactccgtcggcgagatcgccgccgcccatgtcgcgggcgtcctctccctggacgacgcctgccggctggtcgccgcacgggcccgcctgatgcaggacctgccgcgcggcggcgccatggtcgcggtcgaggccaccgagcaggaggtcgccgccgaactcaccgaggcggtgtcgatcgccgcggtcaacggccccacctccgtggtgctcgccggtgcggaggacgcggtgctcgccgtggccgccgtcctcgccgagcgtggccgccgcacctcccgcctcaaggtcagccacgccttccactcggtgctgatggacccgatgctcgccgagttccgcaaggtcgccgagagcctgcgctacgaggcaccccgcatcgccgtcgtgtccaacgtgaccggcgacgtggcgggcgccgacgaactgtgcgacgccgactactgggtgcgccatgtgcgcggcaccgtccggttcgccgacggcgtcaccgcactggccggccgtggcgtccgggtcctcctcgaactcggccccgacgcggtgctgtccgcgctggccgagcagagcgccccggacaccgtgagcatccccgcgcagcgccgcaaccgcgacgaggtggagaccctggtcgccgccctcggccggctgcacgtacacgccgccggaccccgctgggacgccttcttcgacgccgcccccggctcctggctggacctgccgacctacgccttccagcgcggccggttctggccggacaccctgcccaccgccccgcagggcggcgcgaccgcaggggaggccgacggcaccgacagcgccttctgggacgccgtcgcacaggaggacttcggctcgctggagtccgtcctcgacgtcgagggcgacgccctgtccaaggtgctgccggcgctgctcgactggcgtcgccgcacctccgaggagacccagctcgacagctggcgccaccgcatcgtctggaagcggctgaccggcgcggcgatggcccaccgcaagccgctcaccggcacctggctggccgtcgtccccgagggcctcggcgaggacccgtgggtgacgaccgcgctggacgccctgggcacccgggtggtgcgcgccgaggtcggcgccgacggccgggaggccatggccgccaccctgcgggaggccgccggggagggcacccgcttcggcggcgtcgtctccctgctggccctgcgcgagaccgtgcacggcgatgtgccggagggcgtcgccctcaccggcacgctgctccaggcgctcggcgacgccgggatcgaggccccgctgtggtgcgtcacccgcagcgccgtgtccgccaaccgctccgaccgtccgcgtcggccgctgcaggccgccgtgtggggcctgggccgggtggccgcgctggagtacccgaaccgctggggcggtctggtggacctgccggagcgcgccgacgaacgggccgccgccggactcgccgccgtcctcgccggactcgacggggaggaccaggtcgccgtgcgcggctccgccgtcctcgcccgccgcctggtgccggccccggaccgcggacagagcggcgcctgggacccgaccggcaccgtgctgatcaccggcggcaccggagccatcggcgcccatgtggcccgccgactggccaaggacggcgtccagcacctggtgctgctgagccgccgcggagccgaggcgcccggcgccaccgccctgcgcgacgaactccagacgctcggcgccggcgtcaccctcgccgcctgcgacgcctccgaccggggccagctggccgcggtgctggacgcgatcccggacgaccggccgctgaccgcggtgctgcacgcggcgggcgtgctcgacgacggcgtcatcgaccggctcaccccggagcggtaccagaaggtgttccgcgcgaaggtgacctcggcgctgctcctcgacgagctgacccgcgacctcgacctggccgcgttcgtcctgttctcgtccgcctcctccgcggtcggcaaccccggccaggccaactacgcggcggccaacgcggtcctggacgccctcgccgaggcccgccgggtgcagaacctgcccgcgacctccgtgtcctggggcgcctggggcggcggcggcatggccgacggccacggcgcggacgacgcggcccgccgggccggcgtcgccgccatggacccggacctcgccgtggagtcgctgctgcggctggtcaccggcaaggagccgaccgcggtggtcgccgaggtcgacctcgaccggttcgcctccgccttcggcggcgcccggccgagcgccctgctgcgggagttccccggctaccgggagaccgtcgccgccgcggcgccggcgtccccggagggcaccctcgccgaccggctggcgggcatggcgccggcccgccgcctggacaccgtcgtggacctggtacggacccgtgccgcgcaggtcctcggctacccggacatcgacgccgtcggagccgaacggtccttccgggacctcggggtggactcgctgggcgcggtcgagctgcgcaaccagctcggcgccgagaccggtctgagcctgcccgcgacgctggtcttcgaccacccgacgccgctggcgctgggcgagttcgtcctcgcccggctcgcccccgaggaggccgggccggccgtgtccgacgaggagaccgagatcagggccgtactggcctccgtcccgctcgaccgactgcgtgagatcggggtcctggagccgctgctccagctcgccggacgcggtggtggggccggcgacgacgaagacggcgagtccgtcgactcgatgacagtggcagacctggtgcgggccgcgctcaacggccagtccgacctgtagcgcgaagtgatggagcagacgatgaacgcccccgctgagaacgtagttgcggcactgcgcgccgcggtcaaggagaccgagcggctgcgacgccgcaaccggacgatcgtggccgcggcccgggagccgatcgccgtggtcggcatgggctgccgcttccccggcggcgtcgactccccgcaggccctgtgggagatggtcgccggcggcaccgatgtgatctccgagttcccggacgaccggggctgggacctggaggcgctgcgcaccagcggcatcgacgaccgggacacctccgtcagccagcggggcggattcctggactccatcgccgacttcgaccccggcttcttcgggatctcgccgcgcgaggccgtcaccatggacccgcagcagcggctgctcctggagacggcctgggaggcgatcgagcgcgcccgcatcgacgccacccggctgcgcggcacccgcaccggcacgttcatcggcaccaacgggcaggactacgcctatctgctggtgcggtccctggacgacgccaccggggacgtcggcaccggcatcgccgccagtgccgtctccggccggctgtcgtacaccttcggcctggaggggccggcgatcaccgtcgacaccgcctgctcctcctcgctggtggcgctgcacctcgccgtgcagtcgctgcgcaacggcgagtgcaccctggcgctggccggcggcgtcaatgtgatgtcgacgccgggctccctggtcgagttcagccggcagggcggtctcgccggggacggtcgctgcaaggcgttctccgactccgcggacggtaccggctggtccgagggcgcggccgtgctcgccctggagcgcctctccgacgcccagcgcaacggccaccccgtgctcgccgtcatccgcggctccgccgtcaaccaggacggtgcctccaacggcttcaccgcccccaacggcccctcccagcagcgcgtcatccggcaggccctctccaacgcgggcctgaacccggccgatgtcgacgtggtcgaggcgcacggcaccggcactccgctcggcgaccccatcgaggcccagagcatcctcgccacctacggccaggaccgtgaacagcccctgctgctcggctcgatcaagtccaacatcggccacacccagtccgccgcgtccggtgtcgccgggatcatgaagatgatcatggcgatgcgcaacgaggtgctgccgaagaccctgcacgtcgaccggccctccacccatgtcgactggacggcgggcaaggtcgagctgctcaccgagaaccgcccctggcccaccgcgcccgaccgcccccgccgctccggcgtctcctccttcggcgtcagcggcaccaacgcccatgtcatcgtcgagcaggcgcctcagactcccgcccatcagccggaagaggcgccggccgacccgtcggacgaggcaccggccgcgccccgcaccgccggtgtcctgccctgggtgctctcggcgcgctccgccgccgccctgcgcgagcaggccgccgccctcctcgcccatctggacgcccccggtgcccccggcgcgctggacaccgggtactcgctggccaccacccgcgcgtccctggagcacagactcgccgtcgtcaccggcgcggacggcaccgccggccgtgaggcgctcacctcctggctggcgggggaccccgccccggacgcccacgagggccggcccgtcggccgcacccgcagcgccttcctgttctccgggcagggcgcccagcgcctcggcatgggccgtgaactgcacgcccgcttcccggtgttcgccgaagccctggaccaggtcctggatctgctcgacgaggaactcgacgcgagcctcggcgacatcatctggggcgaggaggaagccccgctgaacgagacgggcttcacccagcccgccctgttcgcggtcgaggtcgccctctaccgcctggtggagtcctggggcgtcacccccgacttcgtcgccggccactccatcggtgagatcgcggcggcgcatgtggcgggggtgttctcgctggaggacgcctgccgtctggtggccgcacgtgccgggctgatgcaggcgctgccgagtggtggtgccatggtcgccgtggaggcgaccgaggacgaggtgctgccgctgctgaccgagggcgtggctgtcgccgcggtcaacggcccgacgtctgtggtcgtttcgggtgaggagaaggccactctcgcggtcgccgagcagttggcggcgaaggggcgtcgtaccagccgtctgcgggtgagccatgccttccactcgccgctgatggaccccatgctcgacgacttccgcgcggtcgccgagacactctcctacgacgagccacagctcccggtcgtctccaacctgaccggtaccctcgccgccgacgggcagttgaccagcccggagtactgggtgcgtcatgtccgcgaggccgtccggttcgccgacggcgtccgcgcgctcgccgaggccggcgccgacgtcctgctcgaactcggccccgacggtgtgctcgccgccctggcccagcagtccgccaccgccctcaccgtcccgttcctgcgcaaggaccgccccgaggagcacagcgccgtcaccgcgctggcccggctgcacacggccggtgtcacggtcgactgggccgcgttccacgacggcaccggtgcccgcaccggcgaactgcccacctacgccttccagcacgagcgttactggcccaaggcgaccgcgaccgctgtcgacgccaccggtctcggcctcgcctccgccgaccatccgctgctgggcgccgcgatgtcggtggccggttccgacgaactcctgctcaccggcaccctgtcgctcgccacccacccctggctcgccgaccacagcgtcgacggcatggtcgtcttccccggtaccgggttcctggagctggcggtgcgcgccgccgaccaggccggctgcgaccgggtccaggaactgaccgtcgccaccccgctcgtgctgcccgccacgggcgccgtgcagatgcagatctccgtcggcgccgccggcgaggacggctcccgtgagctgcgcttcttcacccggcccggtgaggacttcgacgccgagtggacccagcacgccaccggccgcatcgcctccggcgagcacgtcctcggcttcgacaccacggtgtggccgccgcgcgactccgaggccgtcgacatcgaggaactgttcgaccgcttcgcctccgacggtctggagtacggcccggtcttccgcggactgcgcgccgcctggcgccaggacgacacggtctacgccgaggtcgaactgcccgactccgtcgaggacgccggtgccttcggtctgcacccggccctgctgcacgccgccctgcacggcaccgccttcctgtccgacgacagtggactgctgccgttcgcctgggagggcgtgtccctgcacgcggacggggcgagcaccctgcgggtgcggatcgcctcctgcggcgaggacacggtggagatcgcggcggtcgacccggcgggtcagccggtcctctccgtggagtcgctgacgctgcgcgccgcggactcgggagccggcgccgcgtcgcgccgtgaggaggccaactcgctgttccgcgtcgactggacgcctcgtacggtggcggcgccggctgcgcccgccacctgggcggtgctgggcgaggacccgttcggcctgacggccgcgctggccggtgactccgaggcggtggcgggagtgcatgctccggccgcgacgctcgaggagctggccgcccggtccggggcggtgcccgacatggtcgccgtgacggtgcgcggtgacgccgatgggggacccgaagccgctcgcgagctgacgcgtgaggtgctggccctggtgcaggggtggctggccgagcccgccttcgcctcctcccggcttgtggtggtcacccgggatgcggtcgccgacggtgagcgtggtgcggtcgatctggccgccgctcctgtgtggggtctggtgcgttccgcccagtccgagaacccgggccggctgctgctggcggatgtcgacgacaccgccgactcgctcgcccgactgccgctgcttgcggggctgttcgacgcggaggagccgcaggccgtcgtccgtgagggcacggtccgggtcggccggctcgcccggctggagtccggtgactcttccgcccgcgccctggaccccgaggggaccgtcctggtcaccggtggcaccggcggcctcgcctccgcgctggcccgccacttggtcgccgagcacgacatcaggcatctgctgctgaccagccgtcgtgggcccgacgccccgggtgccgccgacctcgtccaggcgctggccgaactgggtgccgaggccagggtcgccgcatgtgatgtcgccgaccgtgaagctctggccgcgctgttggcctccgttccggcggagcacccgctgaccgcggtggtgcacacggcgggtgtcctggacgacggcatcttcccctcgctcaccccggaccgtctcgactcggtcatgcggccgaaggtcgatgccgcctggcatctgcacgacctcacccgcgacctcgacctggccgccttcgtcctgtactcctcgacggccggtgtcatgggcagccccggtcaggccaactacgcggcgggcaacaccttcctggacgcgcttgccgcgcaccgtcagtccctcggtctgcccgccacctccctcgcctggggagcctgggaacaaggcgtcggcatgaccggccagttgagcggtcaggacgcacgccgcatcagcgacgccggtgggatgccgctgctgtccgtcgagcggggccttgccctgtacgacgccgccatgctcgccgacgagccgctggtggtgccgctcggtctgggcggcggtggcccgttgcccgccggtgccggcgtccccgcgatcctgcgcggactggtccgcaccggtggacgccgcgcccgggcggccaccgccgcggtcgcccgcgccggactggccgagcggctggccgtcctgcccgaggagcagcgtcggccgttcgtcgtggacctggtgcgtgcggaggccgccgcggtcctcgggcacggttccgccgacgccgtggactcccgtcgtgagttccgtggtctcggcttcgactcgctcaccgcgatcgagctgcgcaaccggctcggcaaggcgaccggtctcaccctccccgccacgctcgtcttcgactacccgacgcccgagcagctcgccgaccatctcctggacgaactcctcggcgccgacgccatcgaggtcttcgccgcctctcagagcgctgccgatgtgcacgacgatcccgtggtcatcgtcgggatgggctgccgcttcccgggtggcgtcggatcgcccgaggacctgtgggacctcctggcgtccggttccgacgccatcaccggcttccccgccgatcgcgagtgggaatcgtcgaggctcgtcgccggtgaggccggcggcgtgtccgcgcagggcgggttcctgagcgacatcgccggcttcgacgcggacttcttcgggatctcgccgcgcgaggccctggccatggacccgcagcagcgcatcctgctcgaggtcacctgggaggccatcgagcgcgccggggtcgacccgaccgccctgcgcggcagccgcaccggtgtcttcatgggcgtcaacggccaggactactccagcctggtcatggggtcccgtgacgacgtcgcgggccacgccaccgccggtctggccgtcagtgtggtctccggccgactctcctacgcgctcggcctggagggtcccgccctctcggtcgacacggcctgttcctcctccctggtgtcgctgcatctcgccgcgcaggcgctgcggtcgggggagtgcagcatggctctggtgggtggtgtcaccgtgatgaccacccccgccaacttcgccgggttctcccggatgggcggtctcgcccaggacggacgctgcaaggcgttctccgactccgccgacggcaccggctggtccgagggcgcggccgtgctcgtggtggagcgcctctccgacgcccgccgcgccggacaccgggtgctcgccgtggtgcgtggctccgcggtgaaccaggacggtgcctccaacggcctgaccgcccccaacggcccctcccagcagcgggtcatccggcaggcactggccaacgccggtctgcgtcccggcgatgtcgacgccgtcgaggcgcatggcaccggcacaccgcccggtgacccgatcgaggcccaggccctgctggccacctatggctccgaccgtgacccgcagcagccgctgctgctcggctcggtgaagtccaacatcggccacacccaggccgccgccggtgtcgccgggctcgtcaagatggtcatggcgatgcgcaacggcgtgctgccgcgcaccctgcacatcaccgagccgtccacgcatgtcgactggtccctgggcgccgtacaggtgctcaccgaggagaccgcctggccggagacgggccgggtccgcagggccggtgtgtcctccttcggcatcagcggcaccaacgcccatgtcatcctcgagggcgcccccgacgagccggtgcccgcgcctgtcgccgaccggcccgtgcccggcgccgtcgcctggcccgtctcggcgaagtccgagggcgcgctcgacgaccaggcggagcggctgcgggagtccgccgacgcgctgcccgcgctcgacaccgcctacaccctggccaccggccgagccgacttcgagcaccgggcggtcctgctcgcagccgacggcaccctcaccgaggtcgcccggggcgtcgccgaaccgcaccgcagtgccttcctgttctccggtcagggcgcccagcgcctcggcatggggcgtgaactgcacgcccgtttcccggtgttcgccgaggccttcgactccgtcaccgccctgctggagagcgaactcgacacctccgtccgtgaggtgatgtggggcaccgacgaaggcgccctgaacgccaccgcgttcacccagcccgccctgttcgcggtcgaggtcgccctctaccgtctggtggagtcctggggagtgacccccgacttcgtggccggtcactcggtcggtgagatcgcggcggcgcatgtggcgggggtgttctcgctggaggacgcctgccggctggtggccgcgcgtgcggggctgatgcaggcgttgccgagtggtggtgccatggtcgcggtggaggcgaccgaggacgaggtgctgccgctgctgaccgagggcgtggctgtggccgcggtcaacggcccgacgtctgtggtcgtttcgggtgaggagcaggccaccctggcggtcgccgagcagcttgccgcgcagggccgccgcaccagccgtctgcgggtgagtcatgccttccactcgcccttgatggacccgatgctggaggacttccgggcggtcgcggagacgctctcgtaccacgagccgcggatcccggtcgtgtccaacctgaccggtgaggtcgccgctgcgggcgtacacacccaccccgactactgggtgcgtcatgtccgtgaggccgtgcggttcgccgacggtgtccgcgggctggccgaccggggcgtgaccgccttgctggagatcggccccgacggagtgctctccgcgctcgccgccgcctccctgaccgacaccgacaccgtcgtcgtgccggcgctgcgcaaggaccgcgacgagaccgtgtccgtcctcagcggtgtcgcccggctgtacgtcgccggggtcgacgtcgactggtcggccccgctgtccggcgccggcgcccgcatcgccgacgtgcccacctacgccttccagcacgagcgttactggcccaaggcggcccccgccgccctggacgccaccggcctcggtctggcctcggccgaccacccgctgctcggtgccgccatgtcggtggcgggctccgacgaactcctgctgaccggctccctgtcggccgccacccatccctggctggccgaccatgtcgtcggcggcatgatcttcttccccggtaccgggttcctggagctggcggtgcgcgccgcggaccaggccggctgtgaccgggtcgaggaactgatgatcgccgcgccgctggtgctgcccgctacgggcgcggtccaggtgcagatctccgtgggcgccgccgacgaggagggctctcgcgagctgcgtttcttcacccggcccggtgaggacttcgacgccgaatggacccagcacgccaccggccgcatcggctcgggtgagcaggtcatcgatttcgacgccacggtgtggccgccgcgcgacgccgaggcgatcgacatcgacggcatgttcgagcgctatgccgcggacggtctggagtacggcccggtcttccgtggactgcgtgccgtctggcgtcaggacgacacggtctacgccgaggtcgcgctgcccgagtcggtcgaggacgccgacgccttcggtctgcacccggcgctcttcgacgccgccctgcactccacggtcttcctgtccgccgagggcgacacccgcagtctgctgccgttcgcctgggagggcgtgtccctgcacgcggacggggcgagcaccctgcgggtgcggatcgcctcctgcggcgaggacacggtccagatcgccgcggtcgacccgggcggtcagcccgtggtctccgtggagtcgctgacgctgcgtgccgcgggcccgggcgacgctgccgagccgcgccgtgacgactccaactccctgctccgggtcgactggaccgcccgtacgctcggggcgcctgccgcccctgccacctgggcggtgctgggcgaggacccgttcggcctgacggccgcgctggccggtgactccgaggcggtggcgggagtgcatgctccggccgcgacgatcgaggagctggccgcccggtcgggggccgtgcccgacatggtcgccgtgacggtgcggggcgacgccgacgcgggacccgacgacgctcatgagctggcccacgaggtgctggccctggtgcaggggtggctggccgagcccgccttcgcctcctcccggcttgtggtggtcacccggaacgcggttgccgacggtgagcgtggtgcggtcgatctggcggccgctcctgtgtggggtctggtgcgttccgcccagtccgagaacccgggccggctgctgctggcggatgtcgacgacaccgccgactcgctcgcccgactgccgctgcttgcggggctgttcgacgcggaggagccgcaggccgtcgtccgtgagggcacggtccgggtcggccggctcgcccggctggagtccggcacctccctcgttccgccggccgggacgccgtggcggttgggctgccgcgccaagggcagcctcgacggtctcgccctgctgccgtacccggaggcggtgacaccgctcaccggccgcgaggtgcgcatcggtatccgtgccgcgggcctcaacttccgcgatgtcctcaacgcgctcggcatgtatcccggtgaggcgggcctgttcggctccgagggcgcgggcgtagtcagcgaggtcggcccggatgtcaccgggctcgcccccggcgaccgggtcatgggcatggtgttcggcggtttcggcccgctgggcgtcgccgacgagcgtctgctgacccgggttcccgacgactggtcctgggagaccgcgtcgtccgtgccgctggtgttcctcaccgcttaccacgcgctgaaggacctcgccgggctgcggccgggggagaagatactgatccacgccggcgccggtggtgtcggcatggcggccatccagatcgcccaccatctcggtgcggaggtcttcgcgacggccagcgagggcaagtgggacgtactgcgctccctcggggtcgccgacgaccacatcgcctcctcgcgcaccctcgactttgagacggcgttcaccgaggtcgcgggggacaagggcctcgatgtcgtcctgaacgcgctggccggggagttcgtcgacgcgtcgatgcggctgctcggcacgggcggccggttcctggagatgggcaagaccgacatccgggactccgacgccgcgtcggacggcatcacctaccggttcttcgacctgggcatggtcgaccccgaccacatccagcagatgctgctcgacctggtcgacctcttcgagcgggacgtactgtccccgctgccggtccgtgcctgggacgtccgccgatcccgtgaggccttccgcttcatgagcatggccaagcacatcggcaagatcgtgctgaccatgccccgggccatggaccccgagggcaccgtcctggtcaccggcggtaccggtggcctcgcctccgcgctggcccgccacttggtcgccgagcacggcatcaagcacctgctgctgaccagccgccgcggacccgatgcccccggcgccgccgacctcgtccaggcactggccgaactgggtgccgaggccagggtcgccgcgtgtgatgtcgccgaccgtgatgccctggccgggctgctcgcctccgttccggccgagcgcccgctgaccgcggtggtgcacacggcgggtgtcctggacgacggcatcctcgcctccctcacccccgatcgcctcgacaccgtcatgcggccgaaggtcgatgccgcctggcatctgcacgacctcacccgcgatctcgacctggccgccttcgtcctctactcctcgacctccggcgtcttcggcagcccgggtcaggccaactacgcggcgggcaacaccttcctggacgccctcgccgcgcaccgtcagtcgctcggcctgcccgccacctccctcgcctggaacgcctgggagcagggcagcggcatgacgagcggactgagcgacgaggacatgcgccgcatcaacgacaacagcggcatgccgctgctgtccgtcgagcggggccttgccctgtacgacgcggccaccctcgccgacgaaccgctggtggtgccgctgggcctcggcggtggcggctcgctgccgcccggcatgagcgtccccgcgatcctgcgcggactggtccgcaccggcggacgccgggccaaggccggcgccgccgcggtcgcccgcgccggactcgccgagcggctggccgtcctgcccgaggagcagcgcctgccgttcgtggtcgacctggtacgggccgaggccgccacggtgctcgggcacggttccgccgacgccgtcgacgcccgccgtgagttccgtggcctgggcttcgactcgctcaccgcgatcgagctgcgcaaccggctcggcaaggcctcaggactcaccctgaccgccaccctggtcttcgactacccgacgccccagcagctcgccgagcacctcctggacgaactgctcggcgccgacgccgcggaggccttcgccgccccccagaccgccgccgccgcgtccgacgacgaccccgtggtcatcgtcgggatgggctgccgcttcccgggcggcgtgggatcgcccgaggaactgtgggacctggtggcctccggcaccgacgccatcaccggcttccccgccgaccgggagtgggagtcgtcgaccatcggcggcgagccgggcgacctgtccgggcagggcggattcctcagcgacatcgctgacttcgacgccgacttcttcggcatcgcaccgcgcgaggccctcgccatggacccgcagcagcgcatcctgctcgaagtcacctgggaggccgtggagcgcgccggactggacccgaccgccctgcgcggcagccgtaccggtgtcttcatgggcgtcagcggccaggactactccggtctggtcatgcgctcccgcgacgacatcgccagccacgccaccaccggcctcgccgtgagcgtggtctccggccgcctctcctacacgctcggcctggagggccccgccctctcggtcgacaccgcctgctcctcctccctggtctcgctgcacctcgccgcgcaggcactgcgatcgggggagtgcaccatggcgctcgccggtggtgtcaccgtgatgaccaccccggccaacttcaccgggttctccaagatgggcggtctcgcccacgacggacgctgcaaggcgttctccgactccgccgacggcaccggctggtccgagggcgcggcggtactcgtcctggagcgcctctccgacgcccgccgcgccggacaccgggtgctcgccgtggtgcgtggctccgcggtgaaccaggacggtgcctccaacggcctgaccgcccccaacggcccctcccagcagcgggtcatccggcaggcactggccaacgccggtctgcgtcccggcgatgtcgacgccgtcgaggcacatggcaccggcaccccgctcggtgacccgatcgaggcccaggccctgatcgccacctacggctccgaccgtgacccgcagcagccgctgctgctcggctcggtgaagtccaacatcggccacacccagtccgcggccggcgccgccggactcgtcaagatggtcatggcgatgcaccagggcacgctcccgcgcaccctgcacgtcaccgagccgtccacgcacgtcgactggtccctgggcgccgtccggctgctcaccgaggagaccgcctggccggagaccggccgggtccgcagggccggtgtgtcctccttcggcatcagcggcaccaacgcccatgtcatcctcgagggcgcccccgagcccaccgcggacgaccgctccgccgaggacaccgtgacgcccgccgtcaccccgtgggtcgtctcggcccgctccgagcaggcgctcgacgcccagctggagcggctgcgcgcccatgccgccgcgcacccggagctgtccggcgccgacatcggtctgtccctggtgacgagccggccctccttcgagcaccgggccgtcctgctggcgggccccgacggcatcaccgaggccgcgcgcgccgaggccggcaccgcccgcacaccggcgttcctgttctccggtcagggcgcccagcgcctcggcatgggccgtgaactgcacgcccgtttcccggtgttcgccgaggtcttcgactccgtcaccgccctgctggagagcgaactcggcacctccgtccgtgaggtgatgtggggcaccgacgaagccgccctgaactccaccgcgttcacccagcccgccctcttcgcggtcgaggtcgccctctaccgcctggtggagtcctggggagtgacccccgacttcgtcgccggtcactcggtcggtgagatcgcggccgcgcatgtggcgggggtgttctcgctggaggacgcctgccggctggtcgccgcccgcgcccggctcatggacgcgctgccccggggcggcgccatggccgccgtcgaggccaccgaggacgaggtgctgccgctgctcgacgacggggtcgccgtcgccgccgtcaacggccccacctccgtcgtcgtctccggccccgaggacggcgtcgaccagctggtcgccctcctggagtcggacggccgccgcaccacccggctgcgcgtcagccacgcgttccactcgtcgctgatggacccgatgctggaggacttccgggcggtcgcggagacgctctcgtaccacgagccgcggatcccggtcgtgtccaatctgaccggtgaggtcgcctccgcgggcacccacacccaccccgactactgggtgcgccacgtccgtgaggcggtgcgcttcgccgacggtgtccgcgcgctcgccgaccggggtgtgaccgccttcctggagatcggccccgacggagtgctctccgcgctcgccgccgcctccctgcccgacaccggcaccgtcgtcgtgccggcgctgcgcaaggaccgcgacgagaccgtgtccgtcctcaccgccctggcccggctgcacaccgcgggcctggacaccgactggagcgcccacttcgccggcaccggcgcccgcaccgtcgaactgcccacctacgccttccagggcacccgcttctggcccgacaccaccgccgctcccggcgacgcgggcggactgggcctggacgccggagggcacccgctgctcaccgccgccacatcggtcgccggctcggacgagacgctgctcagcgggcgcctgtccgccgccgcacagccctggctcaccggccgcacggagaacggcaccaccatcctgcccaccgcggtcctcgccgaactcgcgctgcacgccgccgaggcctgcgaccgcaccaccgtcgagaacctcaccgtcggcgcacccctggcgctcaccgggaaccgcccccagcgcctccaggtactggtgggcgtccccgacgagaccggccggcgcaccctcaccgtgcacacccgcgccgacggcgacgacgccccctgggtcgagcgggccaccgccatgctcaccgacgcgcccgccgccgccacgcccgacaccgtatggccgcccgccgacgccaccccggtcgacgagctgcccgaacccaccggcccgtccgtgctgcgcgccgcctggcggcgcggcggggacgtcttcgccgaggtcgagatcaccgaacagagcccggccgagcaggcgttcgccctgcacccggcgctcctggacaccgcggtccgcgccgccgtcctcctcgaaggagacgggggcggatccggagacgacaccctcgacgccgtcgcctgggacgggctcgtcctgcacgccgcgcaccccgtcctcctgcgggtacggctcaccgccaccggcgacgacacctgggcgctggaggcgaccgacccacagggcggcccggtgctctcggtggcctcggtcaccctgggcgccaccgtcgccgcacccgtcaccggcgcccccgccacggacgacgccgcgctgctcgccctggactgggtcgcgcccgccccggccccgcgctccggcgacagcggcccctggacggtgctcggcgacgcgctgcccggcctggacaccgcgctcgccgcggtggacaacgtcctcgtcacccgcgcggactcgctcgccgaactgctggacagcggggcacccctgccgtcgctgatgctgctgccggtcgagggcggccccaccgcggggcacgacctgcccgccgcggtccgcgccgccaccacgagggtgctggacctgctgcgccgctggacctccgacccccgcaccgcggactcccgcctcgcgatcgtcacccgtggcgccgtcgcggccggccgggaggacgtgaccgatctggcggccgcagccgtctggggcctggtccgctccgcccagtccgagaacccggggtgcttcctgctcctcgacctcgacccggccgacgcggccgaggcgaccgacgccgccgtcctggcctcgctgcccgccctgttcgacgcgggcgagacccaggccgccgtccgcggcggcgcgctcaccgtcgcccgcctcacccgcaccgaggccacgcccgccccggccgccgaccaggtccgtgcctgggaccgcgacggcaccgtactgatcaccggcggcaccggaggcctcggcgccgtactcgcccgccacctggtcaccggccacggcatcaagcacctgctgctggccggccgccgcggacccgacgcacccggggccaccgccctgagcaaggaactctccgcgctcggcgccgaggtcaccgtacgggcctgcgacgtctccgaccggtccgccgtcgacgcgctgctcgccgggctgccggccgagcacccgctgaccgcggtcgtgcacacggcgggcgtcctcgacgacgccacgatcggcaccctcaccgccgaacagctcgacaccgtgctgcggcccaaggcggacgccgcctggcatctgcaccaggcgacccgggccctgccgctcgccggattcgtcctgtactcctccgtggccggcgtcaccggcggccccggacagggcaactacgctgccgccaacacgttcctcgacgcgctcgccgcgcaccgtgcggcccagggcctgcccgccctgtcgctcgcctggggaccgtggggccagggcgccggcatgaccggcacgctcagcgacgccgacctcgagcgcatggaacgctccgggatgccgccgctcaccgaacgacagggcctcgccctgttcgacgcggcgaacggccacgacgaggcgctggccgtggcgatccgggtctcccggtccgccgcggcccccgacgccggcgaggtgcccgcggtgctgcgctccctggtccgcgcccggcgtcgcgcggcggccacggccggcgccgacgggctcacccgccgactggccggcctcggcgccgagcagcggcacgaaaccctcgtcggcctggtccgccaggagacggccggggtgctcggtcactccggggccgacgcggtccccgccgaccgggacttcagccggctgggcttcgactcgctgatggcggtcgaactgcgcacccggctctccgcggccaccggtgtgcggctgccgtccacgctggtcttcgaccacccgacgcccgccgccgtcgcccggcacctggccgactccctgacgggccaggaccgcagcggcaccgccgcatccccgctggcggcgctcgaccggctggaggcggaactgtccgccgacggcgtggacgaggccgtccgccgcggggtcgagggccggctgcggcggctgctggccgcctgggacggcaccggctcggacgggaacggaccggccgtcgaggagcggatcgaggcggcgagcgccgaggagatcttcgccttcatcgacaacgaactcggccggtcgtcggattcctgacccgggcccaccccaactacgtgcgaaacatctgaggaaggttcaacggacgatgccggacgaaagtaagctcgtcgattacctgaagtgggtcacggcggatctccaccagacccgcaggcgcctccaggaagccgagtcgggccgccacgagcccgtggcgatcgtcggcatggcgtgccgcttccccggcggtgtgcgttccccggaggacctctgggagatgctcgccgacggccgcgacgccatctccgggttccccgccgaccgcggctgggacctggagacgctggccggcgacggagcgggcggcagcagcacgcaggagggaggtttcctgcacgacgtggccgacttcgacccggggttcttcgacatctcgccccgcgaggcgctggccatggacccgcagcagcggctgctcctggagaccgcctgggaggccgtggagcgtgccgggatcgcccccggcagcctccgcggcagccgcaccggcgtgttcgtcggcaccaactcgcaggactacgcccacctcgtcctcgcctccgacgacgacatgggcggctacgcgggcaacggcctggccgccagcgtgatgtccggccggctgtcctttgcgctcggcttcgaaggacccgccgtcaccctcgacaccgcgtgctcctcggccctggtcgccctgcacctggccgcccagtcggtgcgctccggcgaggccgacctcgcgctggccggcggtgtcaccgtcatgaccacctcgtccagcttcgtgggcttcagcctccagggcggcctcgccacggacggccgctgcaaggcgttcgccgactccgccgacggcaccggctggtccgagggcgtcggcatgatcctcgtcgaacggctctccgaggcccggcgcaagggccacccggtgctcgccgtgctgcgcggctccgccgtgaaccaggacggcgcctccaacggcctcagcgcccccaacgggcccgcccagcagcgcgtcatccgcgacgcgctggcctccgccgggctctcgcccgccgacgtcgacgccgtcgaggcgcacggcaccggcaccaccctcggcgaccccatcgaggcgcaggccctgctcgccacctacggccaggaccgcgacgcgagccgtccgctgcgcctcggcacggtgaagtccaacatcggccacacccaggccgccgccggcgccgcgggcgtcatcaagatggtcctggccctgcgccacgggctgctgccccgcaccctgcacatcgacgcgccgtccacacacgtcgactgggacgcaggacacgtcagcctgctcaccgaggccaccccctggcccgagggcgagcagacgcgccgggccggtgtgtcgtccttcggcatcagcggcaccaacgcccatgtcatcctcgaagaggcccccgcggccgaggaggacaccgacgccgagcagcggcccgagccggtcgtcccgggcgccgtgccgtggcccgtgtcggcccgtaccgccgacgccctcgacgcccagctggagaaggtgcggccgctcgccgcgtccggcgccgaccccgtcgccgtcggccacgccctggccgtcacccgcacccccttcgaacaccgggcgctgctggtcgccgcggacggcaatctcaccgaggccgcacgcggcaccgtcccctccggcgaccgccccgggctggccgtgctgttctccgggcagggcgcccagcgcctcggcatgggccgtgaactccacgcccgcttcccggtgttcgccgccgccctggacgagaccctcgccctcctcgacgagcgcctcggccactcgctgcgcgacgtcatctggggcgaggaccccgtggccctcgacgacaccggccacacccagcccgcgctgttcgccgtggaggtcgccctctaccgcctgttcgcctcctggggtctgcgccccgaccacctcgccggacactccgtcggggagatcgccgccgtgcatgtggccggcgcactgtccctggaggacgcctgcaccctggttgccgcccgcgccggcctgatgcgggacctgccggccggcggagccatggtcgcgctgcgcgccaccgaggccgaggtgcggccgctgctcgacgagcgggtctcgctcgcctcggtcaacggccccgattccgtggtggtctccggcgccgaggacgccgtcctcgccgtcgccgagcacttccggaagcaggaccggcgcaccacgcggctctccgtgagccacgccttccactccccgctcgtcgacccgatgctcgacgccttccgggacgtcgtcgcgaagctcaccttcggcgagccgtcggtacccgtcgtctccaccctcaccggcgacgtcgtcgccgccgaggaactggccacgccgcactactgggtgtgccacgcccggcaggccgtccgcttcgccgacgccgtgcgcaccctcgtcgacgagggcgcccggaccttcctggaggtcggcccgggcggcgtgctgtccgcgctcgtcggtgagaacacccaggaggccggggtctctgccgtgcccgcgctgcgcaaggaccagccggaggaggcctccgtgctcgccgccctcggcaccctgtggacccggggcaccgcggccgactgggacgccgtgttcgagggcaccctgcccggccgcccggagtccgtggacctgccgacctacgccttccagcgcggccggtactggcccaccgtgcgcgcccgctccggcgaccccgccggactgggcctcggcgccgccgggcacccgctgctcagcgccaccgtcaccctcgccgaggccgacgagtccgtcctcaccggacggctctcgccgctgacccacccctggctcaccgagcaccgcgtcgacggccggatcaccgtccccggcacggccctcgtggagatcgcggtccgtgcgggcgacgagaacggcaccccccgcctggaccggctcgacctgctcgccccgctgaccctcggcgaccgcgacgcggtgctgctccaggtgcgcgtcggccccgaggacgcctccggccggcggacgctctccgtgcacgcccgcccggcgaccgcggacgacgccccctggaccacgtacgcccgtggtgtcctcgcccccgacgagggcgccgaggacaccgccgcggaccttgtggagtggccgcccgccgacgcccgtcaggtcccgctcacggagctggagtccgaaggacgcgccctcggcccgctcttcagcggcctcaccggggtgtggcggcacgagggcgaggtgttcgccgaggccgagctgcccgcaggacccgacagcggcttcggactgcaccccgcgctgctgtccaccgccctgcgcgccgccgccgtcctcgacggcaccacggccggcgagcccgccgccttcgaggggctcaccctgcacgccaccggcgccaccgcgctgcgcgtacggctgagcaccaccgggcccgacaccgtcgacctcaccgccgtcgaccccgcgggcaacctcgtcctgaccgccgagaccgtgcgtctcggcaccccggacggcaccgccgacaccccggccgccaccggccggggcgacctgttcgggctgaagtgggtgcccgtcaaggcctcggagcgcgccaccggcacccgctgggccgtcgtcggctccgacgaactcgacctcggctacgccatgcaccgcgccgacgagaccgtcaccgcctacgcggagtccctcggcggcgccatcggcgacagcggggtcgcccccgatgtgttcctgatcccgctcgcaggcgagaaggacgccggagcggagggcgtgcacgccctcaccacccgggtcctcggctacctccaggagtggctgtccgagccccggctctccggcacccgcctggtcttcgtcacccgccgcgccgtcgccctcgacgacgaggacgtcctggacccggccggcgcggccgtctggggcctggtgcgctccgcgcagaccgagaaccccggcagcctgctgctcgtcgacctcgacgacaccttcctgtccgccggagtactgcccgacgtgctgacgctcgacgagcagcagctcgcggtccgcgactaccaggtccgcgccgcccggctcgcccggctgccgcgcccggccgacgacgcccccgccgccgactggaaccccgacggcaccgtcctgatcaccggcggcaccggcggcctcggcgccgcgctcgcccggcacctggtcaccagccggggcgcccgtcacctgctgctggcgagccgccgcggcccggacgcgcccggcacgtccgaactggtcgccgagctgaccggcctgggagcccaggtgaccgtctccgcctgcgacgtcggcgaccgcgacgcggtcgacacgctcgtcgcgtcggtgcccgccgagcacccgctgaccgccgtcgtgcacaccgcgggtgtcctggacgacgccctgaccggctcgctcaccccggaacaactggctggtgtgctgcgccccaaggccgacgcggccctgcacctgcacgaggcgaccctcggccaggacctcgccgcgttcgtgctgtactcctcgatctccggcgtcatcggcggccccggccaggccaactacgccgccgccaacgcctccctggacgcgctcgcccaccggcgcagggcggccggtctgcccggtctgtccctcgcctgggggccctggggccgcggcagcggcatgaccagccaggtcagcgacaccgacctggggcggatggaccgcggcggcaccccgccgatgagcctcgaggacggcctcgccctgttcgacgccgccctggcccgcaccgagccgatggtcgtgcccacccggatcaatgtcaccggactccaggtccagcagacgctgcccgcgctctggcgcgacctggtgccgcgcccccggcgcaccgccgccgcggaccgctcgcccaagaccgtgctcgacgggctgcgcacgctcgacaccgcgggccgggagaagctgctcaccgagctggtcgtcggcttcaccgcgggcctgctcggccacgccgaccccgccgccgtcgaccccgagcgcggcttcctggagctgggcttcgactcgctggtctcggtcagcctgcgcaaccagctcggcgaactcctcgggctgcgcctgccgacgtcggtggtcttcgacagcaagacgccggtgaagctggcccgccacctcaacgaggaactgggcgacctgtccgcctccggccccgcgtccggcaccgccgtcgccggcaccacggtgcatcccgacgacaccctggtcggcctgttccacaacgcggtgcgcggcggcaagctcgtcgaggcgatgcggatgctgaaggcggtcgccaacacccggcccaccttcgagacgcccgcggacctggaggagctgtccgaggccgtcaccctggccacggggcccggctccccgcggctgatcttcgtcagcgcgcccggcgccaccggcggtgtccaccagtacgcgcgcatcgccgcccacttccgcggcaagcgccatgtgtcggcgataccgctgatgggcttcgcccccggcgagctgctgcccgccaccagtgaggcggcggcccggatcgtcgccgagagcgtcctgatggcgagcgacggcgaaccgttcgtgatggtgggccactccaccggcggttccctcgcctacctcgcggccggggtgctggaggacacctggggcgtgaagccggaggcggtcgtcctgctcgacacggcgtccatccggtacaacccctcggagggcaacaacctcgaccagacgacccgcttctacctcgccgacatcgactcgccgtccgtgacgctcaacagcgcccggatgtccgcgatggcgcactggttcatggcgatgacggacatcgacgcgcccgccacgaccgcccccaccctgctcctgcgcgccacacaggccaacaacggcttcatgctggacacctccgcggtgccggcggacgtggtgcgtgacatcgaggccgaccatctgtccctggccatggagcactcggatctgaccgccgaggccatagagaactggctcgccgaacttccggccggcgaggcctgagccgggtccggccggtgcacgccaccccggccgggtcctcaccgccccaccgcgtccggggcgccgccctcacacggcgtcgccccggacgcgcagcggccccgcagccgggtaaccgcgcacctcaccactctcccaaggagtagcagtcccatggtcaacccgacaccgccgccctctctcgaggatgccgcgccttcggtgctccgcctcagcccgctgctgcgcgagctccagatgcgtgctcccgtcaccaagatccgcaccccggccggcgacgagggctggctggtgacccggcacgccgagctgaagcagctgctgcacgacgagcgcctggcccgtgcgcacgccgacccggccaacgcgccccgctacgtcaagagccccttgatggacctgctcatcatggacgacgtggaggcggcccgcgccgcgcacgccgagctgcgcaccctgctcaccccgcagttctccgcccgccgggtgctcaacatgatgcccatggtggaggggatcgcggagcagatcctgaacggcttcgccgcccaggaacaacccgccgatctgcggggcaacttctcgctgccgtactcgctgaccgtgctgtgcgccctgatcggcatcccgctgcaggagcagggccaactcctcgcggtgctgggcgagatggccacgctgaacgacgcggagagcgtcgccaggagccaggcgaagctgttcgggctgctgaccgacctggccggccgcaagcgggccgaacccggcgacgacgtgatctcccggctgtgcgagacggtcccggaggacgagcgcatcggccccatcgccgcgagcctgctcttcgccggcctcgacagcgttgccacccatgtcgacctgggcgtcgtgctgttcacgcagtacccggaccagctgaaggaggcactcgccgacgagaagctgatgaggagcggcgtcgaggagatcctgcgggccgcgaaggccggcggctcgggcgccgcgctgccgcgctacgccaccgacgacatcgagatcgccgacgtcaccattcgcaccggcgacctggtgctgctcgacttcacgctggtcaacttcgacgaggcggtcttcgacgacgcggacctcttcgacatccgccgctcgcccaacgagcacctgacgttcgggcacggcatgtggcactgcatcggtgccccgctggcccggatgatgctgaagaccgcctacacgcagctgttcacccggctgcccggtctgaagctggccagctcggtggaggagctccaggtgacctccggccagctcaacggcggcctcaccgaactgcccgtcacctggtgacctccccgctgtccggcacccgcggcccccactccccgacgagtgggggccgcacccttttccgcggtcggcgggacgatactggccctgcacgcagccacggacgaggagccccgacgatgaccagcgacaaccgcacaggaccgcccagtttcggcagcgaacgcgagatgctgcgggccttcctcgactaccatcgcgcgaccctcgccatgaagtgtgaagggctcaccgacgaggagctgcggcagcggtcgatgccgccgtccacgctctcgctgctcggtctggtgcggcacatggcggaggtggagcgtgcctggttccgccgggtgttcgaggacaacgacgcgcccatggtctggtccgacaagatcgacttccaggccgcgtacgacgcgagcgggtcgacccgacaggaggcgttcacggcctgggaggccgaggtggccgactcgcgccgcatcgagcgcgagagcgagtccctggaccgggtcggccatcagccgaggtggcgcgaggaggtgtcgctgcggatggtgatggtgcacgtcctcctggaatacggccggcacaacgggcacgcggaccttctgcgtgagggcatcgacggaaccgtgggcgcctgagcccgcgccgcaaccggccccgtggcggccccgccgcgcgccaggactgcgagcgtgctggtcgcagtcctcgacaagcgactgtgaccagcccttccccagcacccctagacggtttcccagggattgctctcgtagcagtccacgagcaggtccgtgaccgccgattcgacggccagtgctgccccgtcgatgctcgcgaccggaagggccggcgagatcgagttcgtcaggaaggcagcgaggaccggggagccggacgcacgggccacgatgtcatcgagccggacctcacgccgctctgaggggatgcccttggcctccagcccccgctgcagcagctgcatcgtgatgcccggcagcaccgcggcctccggccagatcactcgctccccgtcgtagaagccgatgttccagaccgacgcctcgctgatccggccgtaccgatcggtgaacagtacgtcgtcgtacccggagagcaccgcctgccgtcggccatgaatgagcccgaacgtccccacgtgtttcacatgaggcaggtcccgttcgtactccaccgcccgcagtcgcatcggctcgctctgcgcctcagccggggcgctcgtcgccaccgccagctccggctccacggcctcgccgcgcagcacggcatccagcctccgggagaagacggtcacccggaccgataccgcctccgggcccccttcgagggcgtgccgcacacatccccgcacccgctcggcgtccaggcccctgccgaacaacccgcgcgcgctctcgtccaaccgtctcaggtgcagatccagcccccggaccgcaccgccccgcacctgcatcgaggtgaaatgcgcgtagctgacccgcatgagcaccttgaggtcctcgctgtccgcctcgacaccgttgacttccacccggtcgatgccaacccctccctcacccacacccagcccggctgccatgtcccgaccaccttatgtgcgcgccaagacgttcaaaagtgggccagatcgcctttcgcaacaggatctgggtgcgctgtcgcggcaggttggtgacgcggcaggcccggaggacagcgtcaggagcgggcgcctggctcctgccgtgacacgcgacgaacggagcttcgcccttccgcaacccggcagcgtcgctcgccggacgctgccgaacaccgcacaagcgcgtgcggcccggccgatggaaggattccgtgcgtgaacgatgccggtgaagtggtctgccgtccctccagcaagcgccctctgtggttcttcgtcggactcggtgcggccggggcgctcctggccgccgggcgtgcggcgttcgggggcgggttcctggacgtgggggtgggcgtcggcctgctccttgccctggtggggatcgcggctctccacgcggtgaccgcccaggtgcgcgccgacgcgtacggcctgcactcccggacactgttgcgccgccggagcgtgccgtggcgcgatgtcgccgacctgcgcgtacgtctgaagtacataaaaaacggagggaagtcccgtcgcgtcggcctggtgctgcgcgacggacacaggcggcacctgcccctcccgacgtccgtgaagtcccacgatccggacttcgacgcgaagctggagggactccgcgcgctccaccgccactacggaaccccggagtcgagccatatcgccgtcgtctctcccggcaccgccggggccggctgggtcgggtcgctgatcgcgtgcgcgctgctgctcgtgagcgcgggcgtggcggcggtattcgtgccgagcgccgggtcgcacatgcgggagtggaagtcggcgaccccgtgcaccgccgagagtcccgtcacggaacgtggcgaatgcctcaccaccctgccggccgtgatcgcgcggaccgacgcccaccagcccaagcagccgagctggctgtacttcatggacggccggccggtgaagcgagtccgggtttcgtacgaggccgccgaggaattccagtccggcgacaacgtcgaactgaccttctggcacggtcaggtgatgaaggtcgccggagaaggccatgtttggcgggaacacatcgccggcgccggggacgtggcggtcgtcgctgccgtgctcaccctcgccgcgggctaccctgcggcccgggtactgctgcgcctgcgcgggcgccggctgcccgacgacgaggtccttccgtcggaacttccgttcgcgggcgcgctcgtcggtacggccctgtggctgctgccgctctgctacgtccaccccctgtccctgctcgactcccccgtggcgatgacatgggcggccgcgggctcgctggtctcgctgggcctgttcggctgggcctggcacgccactcggatcattgtgcccggagcgatcggagcgatcggagcgatcggagcgtccgaagcatccgccgggaaagcgggggaagaagcgtccaccaagaaggcgggggaaggggaagaggaggtgttcctggtcgcccgtttcctggaggacaccgactacaacccttacggcttcggcacccacatcgccctgggtggtggcaccctcgcggtgacgcccggcccgggccggtacggagcgcaccgcataccggtggaacggctgatcgtcaagggcgtgcggcgcgcccggggtggcgacgacgacaccgtccccgcaagctggcacatcgccgaactcgacgacgcaggcacgaccgttcgcctcgcggcggccccggccgacctggcccgcatcatccgcgcgttggactccgcggaggcctccgtgaactctgcggaccccaagccgtgatgtcctggggcgcctgcgaggatcggccttcctgatcggcggccggacaaggccggacagcggagacacctcagggcgtgagtgccggcgatgcggcctcccacgcaaaccctgtggaacgcagcgcggcggagccggcgaagggttgatgtcctgctgatcggatgtggctgtcgggtccaggtgagcccgtgcaacctctcccgccactgcccaccggtctcgagctgccggactcgcccccgaactgctgccgccgccgaacgtggtgcgggccgtacccgccggtcggcaggtacggcccgttgaactcccactacggctcggctagttctccacgacggtgatcgccatcgaggggcagaccaccgcggcctcccggaccgtctccaccgtggacgggtccgacggctgtgcgaggacggtcaccaggccgtcgtcgtcctggtcgaacacgtccgacgcggtcaggacgcactgtccggcaccgacgcagcgatcgacgtccacgttgacgtgcaccatcattcctccagggtcgttccagcgggccggccgtcgcagcggacgggcccgtcgctcagctccatgtgacgggcagctcgtgcaggccgaacagcacgccgtcgtacttcaggggcaactcctcgaccggcaccgccagttcgagggtcgggatgcgctcgaacagggtacggtaggcgatctccatctccacccgcaccaggttctgcccgaggcactggtggacgccgtacccgaacgccacgtgcgagcgcgcggaacgctccgggttgaactcctcgggcttgtcgaagaccgaaccgtcgtgattggccgcggcgatcagcggcacgatgccctcaccggccttgatcagctggccgccgatctccacgtcctccacggccacccggaaggacaccaggtcggcgaccgagtagtaccgcagcatctcctccacgatgcggtcgtcgccgatccactgcgggttggtcagcaactggacgacgcccaggccgatgttgttcgccgtggtctcgtgccccgcgatgagcaggagcatcgacaccccggacagctcctgccggttcatggtgccggcctggagcagccggctgatcaggtcctcgccggggcggcggcccttgatctggatgagccggttcagataacgcagcagcgccttggaggcctcgtcacgttgctcgtcggtggagctgagggtcaccaggacctttgtcttcgcctcgaagaagtcccggtccgccttgggcaccccgagcaggtcggagatgaccagcgacggcacggacagcgcgaagtccgcgatcaggtccgccgagttgcctgcggcgagcaacgcgtcgatccgctcgtccacgatctgctggatcgccggacgcagctcccgcaccttgcgcacggtgaactccgggatcagcgtcttgcggaaccggtcgtgctccggggggtccagcgccacgaaccagcccgggatctccgactgcgagggggcgccgccggtgcgcctggccctcgggaagccggggcgggaggggtccgcgctgatccgggggtccgtcagcacggtccgcacgtcctcgtgccgcgtcaccagccacaccgggccgcttgcgggcagttcggagcggaccaggccctcgcccgcacggtagtcggcgtactccggcggcgggaacggacgtcccggccggcgcagcgggaaggcgacggggcagcccggcgcgaaggtcgtcatctcggtctcggcggtcatcacaggactccgtagaaggcgcgtacccggtcggtgatgaactcctggtcctgcgcggtcagcccggtgtgcgtcggcagatagaacccgtcctcgctgaaccgggaggcgttcagcgacggccagttcgcgtcgtagtagcccggctgacggctcatcggcttgaagaacacccgggtctcgacgccctggtcggccaggtacgcgcgcagctcctcgctgcgctcggcccgcaggtcgtacatccacagcacgtcgcgggccggcatcagcgtgatgccgggcacgtcccgcagcgcctcgtcgtagcgcttctcgatgtcgcggcgggtcgcgaggatggtgtccagctgctcggtctgcgcgagcgccaccgcggcctgcatgttggtcatgcggtagttgtaggccagcttcttgtgcaggaagctgtggtccttggtgaacgccatggcccgcagatgcgccatctgctcggccagatgggcgtcccgggtcacgcacaccccgccctcacccgccgtgatgatcttgttggcgaacagcgagaagcaggcgatgtcgcccaccggccgcaccccgtgcgcctccgccgagtcctcgacgacccgcaggttgtactcgtacgccaggttcatgatggcgtccatgtcgcactgccggccgtagatgtgcaccggcatgatcaccttggtgcgcggggtgatcttctcctcgatgcgggagacatcgatgttcaggtcgtcgccgcagtccacgaacaccggggtcgcccccgtgtaggtgaccgcccaggcggtcgcgatcatggtgaactccgggacgatcacctcgtcgccggggccgacgcccagcgcccgcagcgccagcgtcagcgcggtggtgcccgaggaacaggcgacaccgtgcggcatgtcgttgtaggcggcgaacgcctcctcgaactgccggacgtaggggccctgcgacgagatccagccgccgctgacggcctccgtcacatactccagctcgcggcccttcagccacggcatggagaccggatacgtataggacatccttgacttcctcggtcagtcgtttgccaggacggggaggccgagcagcaggtccgcggcggcggtacgaccacccgcggccttcagcaacccggcgtagtactcggcgcgttcggtgaaggaggactcgttcagcacgcgggtgatcttgtcgaggacgtcgtccgtgtccacggtctcgggacggtcgacggtcaggctcacgccgaagtcctggccgcgcacggcctggtcgtcgcagtccacccacaacggccgtaccaccagaggctttccgaagtacaggccctcgtggtagccgttgccgccggcgtgcgtgaagaacaccttgacgttcgggtgcgccagcacgtccagctgtgagggcacccagctctcgatccgcaggttcgcgggcagttcctcggccgggggcagcagatgctgctgctcgctcggcagcttccacagcacctggtggccctcgccctcgaggcggcgggccacctccaccagcgaggcgacctgctcccgggtgagccgggtgatggtgccgaagcccatgaagaccacggacttctgctccgtcagccagtccgagaggccctcgtcgtccggcgcctgcggcagcggcggcaccagcgtgcccaccagccgtaccttctcgtgcatcgggaacgggtagtccagctccgccaccgagtagcacagcacctgctcggcgtggtcgatgcgcgccatcatctgccgcgcctcgggggagatgcccagctcgccgcggacccggttgtcctcctcgacgatctcccggatctccttcgtcatgaacatcccgagcgtgcgcacccggaacagctcgttctccacccgctgggcgagcgacatcttgccgggcaggcccgagtgcggcaccgggaacccggacggcgtgtacgacttggcgaacggcacatgcgaggtcagtacattgctcggcaggaacggcaccccgagcacgaacgggatccccttggtgatcgccagctcgtacccgaactggcacatgctctcgatcaccatcagggcgggctggatctcctcgacggccttctccagcgcccggtacttctccacccgggtctcgggcgcgaaggagtgccggatgaccgcgcggtgcgccttgaagcgggagcgctgcgtcacctcggcataggtctcgtcgtcccaggtgaccgccgacatctgcgacacggtgtcaccgagcgaggcgaactgcagctcactgtccgccgacgccgactcgatctggtcccgcgccttctcgtccgtggcgaaccacagatcctccacaccacgccgggacaattcctcggccagtacgagcagcggattgagaagaccgctttcggcataactgacgaacaggatcggccgcctgtgcgcgcccatggacaacaccctccggaaagtggcggaccgcgcaggcccgcacgcctcgcgtccacccaccgaaagtcgccggatgatcgcattcgccgacgccgccgcctgtcgcgcacgccgtcacaggacaccgccgaccccgccgctcggcacaccgtgccgagcgggagaagcgtgccgctgacttcatctcacgccagggtccggggaatccctagtcttcgccccggagcgggtgttttcgtgtccgtccgggcgcggcccggagccgaacactgagattccttagggaaaagtacagccttcgcgcccatacgatccggcaggcagaggtggcccaggaccgacttttccgcaggggtgagcgaggcatgacgatcggagccaacgacgatccggtagtggtcgtcggaatggcctgccgcttcccgggaggcgtcgaggggcccgaggacctgtgggagctggtccgcgacggccgcgacgccaccgggccgttccccggcgaccgcggctgggacctggccgccctgaccggcgacgggccggaccacagcgtgacccaccgaggcggattcctcgccgcggccgccgacttcgacgccggcttcttcgggatgtcgccccgcgaggccgtctccaccgacccgcagcagcggctcgtcctggagacctcctgggaggccctggaacacgccggcatcgacccgcacaccctgcggggcacccgcaccggcgtcttcgtcggcaccaacggccaggactacgcgaccgtcaccaacgcctcccgcgaggacctcaccgggcacgccctcaccggtctgtcgccgagcatcgcctccgggaggctcgcctacttcctcggcctcgaagggcccgccgtcaccctcgacacggcgtcctcctcgtccctggtcgccctccactacgcgctgcgctcgctcaggtcgggggagtgcaccaccgcgctggccggcggcgtcaccgtgatgtccacaccggtcgggttcatcgcctacacccggcagggcggactcgccgccgacggccgctgcaaggtcttctccgacgacgccgacggcaccacctgggcggagggcgccggcatgatcgtgctggagcgcctgtccaccgcccgcgccgccgggcaccgggtgctcgccgtgctgcgcggctccgccgtcaaccaggacggcgcctccgacggtctcaccgcccccagcggaccggcccaggaacgactcgtccgcgaggccctcgccgacgccggactcggacccgccgacatcgacctcgtcgaggcccacggcaccggcacccggctcggcgaccccatcgaggcccgggccctgctcgccacctacggccaggaccgcgacggcggacagccgctgcgcctcggctccctgaagtccaacatcgggcacgcccaggcagccgccggcatcggcggactcatcaaggccgtccaggcgctgcgccacggcctgatgccggagaccctgaacctctccacgcccacccggcacgtcgactggtcggccggcgccgtcgaactcctcaccgaggccctgccctggcccggcaccggccgcccgcgccgggccgccgtctcctccttcggcatcagtggcaccaacgcgcatgtcatcgtggaggaagccccgacgaccgaccccgccgccgcggtccccgccgggcccgcccaccgggacgtggcctcggccgccgactccgccgcgcggcccgctgccctcgccggggagcccgccgacacctctgctcccgccgctgtcgacgccggcccggccgaccgcccggtcacccccgccgctcggctcgccgccctcgtgcccgcggccgacgccgtcgcgtggccggtgtccggggcctccccggaggctctcgacgcgcaggtcgagcggctcacctccttcgtccgggaccaccccggcgccgatccgctggacatcggtcactcgctggccaccgggcgggcggcgttgcggcaccgtgcggtgctggtgccgtccggtgacggtgtcgtggagatcgcccgcggtgaggccgccccccgcaccaccgccgtcctcttctccggacagggctcccagcggctcggcatgggccgtgaactcgccgcccgcttcccggtcttcgcgaaggccctggacaccgtcctggccgccctcgacccccaactcgagcgtccggtgcggtccgtgatgtggggcgaggaccccgccgaactcgaccgcaccgggtggacccagcccgcgctgttcgccttcgaggtcgccctgtaccggctcgccgagtccttcgggctgcgccccgacgccgtcggcggccactccgtcggcgagatcgccgccgcgcacatcgccggagtgctctcgctggaggacgccgcgcgtctggtcgccgcccgcgccaccctgatgcaggccctgcccgagggcggcgccatgtccgccgtcgaggcctccgaggacgaggtgctcccgctgctcgacggcgatgtctcgctcgccgccgtcaacggccccaccgcggtcgtcgtctccggcgccgaggacgccgtggagcgcgtctccgcccacttcgctgcccaaggccgccgcaccagccgcctcgcggtctcgcacgccttccactcgccgctgatggagccgatgctcgacgccttccgggacgtcgtcgccggactcaccttccatgagccgacgctgccggtgatgtccaacctcaccggtgaacttgccggtgccgagatcgccacccccgagtactgggtgcggcatgtgcgcggtaccgtccgcttcgccgacggcgtgacggccctgcgggaacacggcaccgacctgctggtcgaactgggccccggcagcgtcctgaccgccctcgcccgcaccgtcctcggcccggacaccccgggcgcccctgtcgacgtggtgcccaccctccgcaaggaccagcccgaggagagggctctcaccgccgcgctcggccggctccatgtcctcggcgcgaccgtcgactggtccgccctctacaccggcaccggagcccgccgcaccgacctgccgacgtacgccttccagcacgcgcggtactggcccgccccgggccggcccggcaccggtaccgcgggcggcgggcatccgctgctcggcccggccgtggaactcgccgacggcggcacggtgtcgggcgccacactgtccgtcgccacccacccctggctcgccgaccatgtcgtcgccgggcgcgtcctgctgcccgccgccgtgctcgtggaactcgccgtacgcgcgggtgacgacaccggatgcgacgtcctgcacgaactcgccctcgtcgaggcgccggtcctggaggccggcgacaccctggacctccaggtccgggtcggctccgccgacgaggccggccggcgcaccctcaccgtccactcccgtcccggcaactcccccgccgagccctggacccagcgggccggcggcctgctcggcaccgcgccccgcaccgcggcggcccccgacacctccttcgccgtcgcctggcccccgccgggcgccgaacccctcgacctcggggaccactacgagcggctcgtcgacgacggcttcgacctcggccccgccttccgcggtctgcgcaccgcctggcgccacgacggcgcgttcctcgccgaggtcgaactcccggccggcaccaccgacgaccccggcgcctacggagtgcaccccgcgctcctcgacgccgcccggcacgccgccctcaccaccaccggcacactcccggtcgcctggcacggggtgcggctgcacgccgtcggcgccaccgccctgcgggtgcggatccactccgccgacgacggtgccctgaccctgaccgccgccgatgtcaccggcgccccggtgttcaccgccgaggccgtcgtcgtacggcagctcaccgagcaggagcgcaccgccccccggccgctcacacgcgcctggcaccaggacaccgcgaccccgcgccgcacccggcccgtcgccgcggcccccggcgccgccgccgagccgtccgcctcctcggcgccggacagcttcgccgccgaggccgcggccctggcccccgccgagcgtgaacgccggctcatcggcctggtacggacccaggcggcggcggtcctcggccatcagggcccggacgcggtcggaccccgcgcggtcttcaaggagctgggcttcgactcgctggccggcgtggaactcagtgaccgcctcaccgcgctcaccggactgcggctgccggccaccctcgtcttcaacttccccacccccgagctcgccgcccggcgtatcggggaactcctcgtcgtatccggctcctcaccgcagggatcgtgcgacgacgaactcaccaggttcgaggccgtcgtgcagaccctgtcggccgacgaccccggacgccaggccgtcgccgaccgcctggacgcactcgtcgcctcgctccggcggaattccgccccgcaggagaacttctccgacgaggacatcgaatcggtgtcggtcgacagactgctcgacatcatcgatgaagagttcgaaatctcctagcggatctacagctttcgttcgcgacccgatgacgaggacggacccgatgccggaaccccagcagaaccagcaggaaaaagttgtcgactaccttcgccgggtcaccaacgatctgcgacgcgcccgccgccggataggcgagctggaatcccgcgacaacgagcccgtcgcgatcgtcggaatgagctgccggctgcccggcggagtgagttccccggaatccctgtgggaactcgtggaatccggcggggacgccatctccggatttcccgccgaccgcggctgggacatcgaggcgctcaccggaaagggcgagggaagcagctccacccacgagggcggattcctctacgaggccaccgacttcgatgccgggttcttcggcatatcgccccgcgaggcgaccgccatggacccgcagcagcgcctgctgctcgaagtctcctgggaggccctggagcgggccggcatcgcccccacctccctgcgctccagcgcgaccggcgtcttcgtgggctcctaccactggggcgcaccctccgcggacaccgcgaccgaactccacggccacgccctgaccggcacggccgccagcgtgctgtccggccggctctcctatgtctacggtctcgagggcccggccgtcaccgtcgacaccgcgtgctcctcctccctcgtcgccctgcacatggcggcccagtcgctgcgcaacggggagtcctcgctcgccctcgtcggcggtgtcaccatcctcgccgagccctccgtcttcgtggagttcagcgcccagggcggtctcgcccaggacggacgctgcaaggcgttctccgactccgccgacggcaccggctggtccgagggcgcggcggtgctcgtcgtggagcgcctctccgacgcccgccgcgccggacaccgggtgctcgccgtggtgcgtggctccgcggtgaaccaggacggtgcctccaacggcctgaccgcccccaacggcccctcccagcagcgggtcatccggcaggcactggccaacgccggtctgcgtcccggcgatgtcgacgccgtcgaggcacatggcaccggcacaccgctcggtgacccgatcgaggcccaggccctgatcgccacctacggctccgaccgtgacccgcagcagccgctgctgctgggctcggtgaagtccaacattggccacacccagtccgcggccggtgtcgccggactcgtcaagatggtcatggcgatgcacaacggcacgctcccgcgcaccctgcacctcaccgagccgtccacgcacgtcgactggtccctgggcgccgtccggctgctcaccgaggagacggcctggccggagaccggccgggtccgccgcgccgccgtgtcctccttcggcatcagcggcaccaacgcccacgccgtcctcgagcaggcccccgtgcccgccgaggacgccgaggacgccgggaccgaggacgccccgcagcagccggccaccgtgccgggcgccctgccgtggctgctctccgcccactccgggcaggcgctttgcgaccaggccgccgccctcgtcacccgcctcgacgccgaccccgagccgcgcccccaggacatcggccagggcctgctgacctcccgggccctgctggagcgccgggccgtcgtcgtcggcaccggccgcgacgaactgctcaccggcagccgcgcgctggccgagggccagtcctggaacggactcgccgagggcaccgccgacgtcgacggccggaccgtcttcgtcttccccggtcagggctcccagtgggtgggcatgggtgcccgactcctggacgaatcaccggtgttcgcggagcgcatcgccgagtgcgccgccgcgctcgccgagttcaccgactgggacctgctcgacgtcctgcggggtgccgagggtgctccgacgctggagcgcgtcgatgtcgtccagcccgcgtcgttcgcggtgatggtgtccctggccgccgtgtggcgtgcgcagggtgtggagcctgacgcggtggtcggccactcgcagggtgagatcgccgccgcggtggtctccggcgccctgtcgctgcgtgacggtgcccgggtggtggcgctgcgcagccaggccatcggccggacgctggccgggcggggcgggatgatgtcggtggcgctgccggtcgccgatgtggaggcccgtcttgaggcgtttggcggacgggtgtccgtcgccgccgagaacggcccccgctcctgtgtggtctccggcgaacccggcgccctggacgagctgtccgaactgctcaccgccgagggcgtgcgggtgcgccgcgtccccgtggactacgcctcgcactcccaccatgtcgaggacctgcacgacgagatcctccagctgctggccgaggtcgcgcccaaggcgtccgaggttccgctgttctccacggtcaccggtgactggctggacaccaccgtcatggacgccggctactggttccgcagcctgcgcggccgggtgctgttcgcggacgccatccgcggtctgctcgacaccgagcaccgtgccttcatcgaggtcagctcccacccggtgctcgccatgtccgtccaggacatgatcgacgacgccggagtggtcggcgtggcctccggcaccctgcgccgtgacaacggcggcctcgaccgcttcctgctgtccgccgcccaggtgttcgtgcgcggcgtccaggtcgactgggccccgctgttcgagggcaccggcgcgacccgggtcgacctgcccacctacgccttccagcacgagcggtactggaacgaccgtgccgtcgtggaccgttcggcctccgcgccgatggacgccgagttctgggcggccgtcgagcaggcggacgtctccgccctcaccgaggcgctcggcaccgacgaggagtccgtcgccgcgatcctgcccggcctcgcctcctggcgccgcacccgcggcgagcgctccaccgtcgactcctggcgctaccgggtcgtctgggagccgctggcccagatcccccgcgccaccctggacggcacctggctgctggtgtccgcaggcggcgtcgacgacgacgtcgccgaggtgctggaggcgggcggcgccgaggtgcgccgtctcgtcctcgacgagacctgcaccgaccccgccgtcctgcgcgagcacctcacggacacggacggcctcaccggcatcgtctccgtcctcgccgacgccgaggagggctgcgtccgccaccccggactcaccctcggactcgccctgtcggtgtcgctggcacaggcactcggcgaggccgacgtcaccgccccgctgtggttcctgacctgcggcgccttctccaccggcccgtccgacaccgtcacccggccgctccagagccagatcgccggtctcggctggaccgtcgccgtcgaacacccccaccgctggggcggcggcgtcgacctgcccgaggcgctggacgcccgcgccgcccagcggctcgtcgccgccctcgccggcgcgctcggcgacgacgaccagctcgccgtgcggcccgccggtgtcctcgcccgccgtatcgtccgcgcgagcggcgacacccgccgcaaggcccgctcctggaagccgcgcggcaccaccctggtcaccggcggctccggcaccctcgcccccggactcgcccgccacctggccgcccagggcgccgagcacctggtgctgctcagccgccgtggcgccgacgcccccggcgcggccgaactcgccgccgaactccaggcggcgggcaccgaggtgcggttcgcggcctgcgacatcaccgaccccgacgcggtcgccgccctgctcgccgacctgaaggccgagggccgcaccgtccgcacggtcgtgcacaccgccgccgtcatcgagctggccgccctcgccgacaccaccgtcgacgcgttcgccgacgtggtgcacgccaaggtgaccggcgcccggatcctcgacgaactcctcgacgacgaggagctggacgacttcgtcctgtactcctccaccgccggcatgtggggcagcggcgtgcacgccgcgtatgtcgcgggcaacgcctatctgtccgcgctcgccgaacagcgccgcgcccgcggcgcccgcgccacctccatccactggggcaagtggcccgacgacctcgaacgcgagctggccgacccgcaccagatccgccgcagcggcctggagtacctcgacccggagctggcgatgaccgccctgacccgggtcatggaggacgacgagaccgtcatcggtctgatggacatcgactggggcacgtaccacgacgtcttcaccgccggacggccctcgcacctgttcgaccggatccccgaggtggcgcggctgctggccgaccgggccgcgcccgccgcgacggccgttgccaccagcggcctggccgcccgcctccagggcgtgtccgccgccgagcaggaccggatcgtgctctccgtggtccgcgaggagaccgccgcagtcctcggccacgcctccgccgagaccgtcccggaacgccgcgccttccgcgacatcggcttcgactcggtgaccgccgtcgacctgcgcaaccgcctggtcgccgcgaccgggctcaccctgccgtccaccatggtcttcgaccaccccaacgccgtcgcgctcgccacgttcctgaaggccaccgcgctcggcaccaccggcacggccggcgaccggcccaccgccgcggtcaccgcgggcgccgacgacgacctgatcgtcatcgtcggcatgagctgccgcttccccggcggcgcgaacaccccggaggagctgctgcggctcgccctcgacggcgccgacgtcatctccgagttccccgccgaccgcggctgggacgcccacggcctctacgacccggaccccgaccggcagggccgcacctactccgtgcacggtggcttcctgcacgaggcggccggcttcgacgccgggttcttcggcatctcgccccgcgaggcgctggccatggacccgcagcagcggctgctcctggagacctcctgggaggtcctggagcgggccggcatcgaccccgagtcgctgcactccaccgccaccggcgccttcttcggcgccagctaccaggactactccagcagcgtgcagaacggcaccgagggcgccgaggtgcacatgatgaccggcaccgcggccagcgtgctgtccggccggatctcctatctgctgggcctcgagggccccgcggtcaccgtcgacaccgcctgctcctcctccctggtcgcgatgcacctggcctgccagtcgctgcgcagcggcgagagcagcctcgcgctggccggcggcgcggccgtcatggccaccccgcacgcgttcatcgggttcagccggcagcgcgcactggccgagaacggccgctgcaagccgttcgccgagggcgccgacggcatgaccctcgccgagggcgtcggcgtcgtcctgttggagaagctctccgacgcccgccgcaacggccaccgcgtcctcgccgtcgtccggggctccgccctcaaccaggacggcgcctccaacggcctgaccgcccccaacggcctctcccagcagcgcgtcatccgtcaggccctcgccaacgccggactcggcaccgccgacatcgacgtggtcgaggcgcacggcaccggcaccaagctcggcgaccccatcgaggccggcgccctgctcgccacctacggccagggccgcgacggcggacagccgctgctgctgggctcggtgaagtccaacatcggccacacccaggccgccgcgggcgtcgccggtgtcatcaagatggtgatggccatgcacgccggtgaactgcccggcaccctgcacatcgacgagccctccagccatgtcgactggacctccggcgccgtcaccctgctgcgcgagcgcaccgagtggcccgccgtgggggaccgcccccgccgggccggcatctcctccttcggcatcagcggcaccaacgcccacctcctcctggagcaggccccgcccgccgaggagcccgccgaagaacccgccggtgacatcgagggcgccccgctcgtcccctggctgctgtccgccaggtccgcgcaggcgctgcgcgagcaggcgtcccggctgctgaccgcgaccaccggcaccgacgtcaccgtcgagcccgcccgcgtcggacacgccctggccaccaccaggaccgccttcgaccaccgcgccgtcgtcctcggcagccggcccgaggacttcacccgcggcctggaggccgtggtgtcctccgcgtccgcccccggcgtcgtcaccggcaccgccggaccggactccacgggacccgtcttcgtgtacccgggccagggctcccagtggtgggggatgggccgcgaactcctcgccggctccgaggtgttccgcaccgccgtcgacgactgcgccgcggcgctcgccccgtacaccgactggtcgctgcacgaggtgctggccggcgagggcgacccggagctcctggagcgcgtggacgtcgtccagcccgccctgttctcgatgatggtcgcgctcaccgcgctgtggcggtcgcacggcgtcgaaccggccgccgtcgtcggccactcgcagggcgagatcgccgccgcccatgtcgccggggcgctcagcctggaggacgccgccctcgtcgtggcgctgcgcagccaggccctgccccagctgtccgggctcggcggcatgatgtccgtctccgcgcccgccgcacgcgtcaccgaactccttcagccctggggcgaggcgctgtccatcgccgccgtcaacggcccctcctcggtgatcgtctccggcgacgccgacgccctcgacgaactcctggagacctgccgcgagcagaacgtacgggcccgcaaggtgtccgtggactacgcctcgcacggccggcacgtcgaggccgtgcgcgacgaactcgcccgcgtcctcgcccccgtcagcccccgcacccccgaggtgccgttctactcgaccgtcaccggcgcccggctcgacgaggcggccttcgacggcacctactggtacaccaacctgcggcagacggtgctgatggaggacgccacccgcgccctcatcgcctccggacaccgggtgttcgtggagatcagcccgcacccggtgctggcggccccgatccaggagacacaggagagcgttcaggaggagctgaccgagaccggcgccgtcctcggcacgctgcgccgcgacgacggcgaccgccgccggttcctcacctcgctcgccgaggcgcacacccatggcatcgccgtcgactggaccaccgccttcgaccgccccgcgtacgcggccgtcgacctgccgacctaccccttccagcggcaggacttctggcccgagccgaagaccgccgcccccgcgggcaccgccgacgcctccgacgccgccttctgggaactggtccagaaccaggacctcgacgcgctcgccggtgaactcggcatccccgccgacgacggcgccgcccggctcggcgacgtactgcccgcgctgtccgcctggcgcacccgctccctcacccgctcccgggcggacgccctgcgctaccacatcgagtggaaccgcgtcgccgaacccggcaccgcccggcccgccggccggctgctcgcggtgatctcgccggaccacgcgggcgccccctgggtcaccgccgtcctcgacgcgctcggcccggacaccgtccgcttcgaggccaagggcaccgaccgggccgcctgggccgcgcagctcgcccagctccgcgaggacgagggggagttccacgccgtggtctcgctgctcgccgccgccgaggcactccacaccgaccacggttcggtgcctctcggcctcgcccagaccctcctcctcgcccaggccctcggcgacgcgggactcaccgcgcccctgtggtgcctgacccggggcggtgtcgccgccggccgcggcgatgtcctcagcagccccgtgcagggtgcgctgtggggcctcggccgggtcatcggcctcgaacaccccgaccggtggggcggtctgatcgacctgccggagacggtcgacacccgggccgccgcccggctcaccggactgctcgccgacgcgggcggcgaggaccagctcgcgatccgcggttcgggcgtgctggcccgccgcctcgcccacgccgccccggccgtacccggctccgggaagcgcccgccggtccacggctccgtcctcgtcaccggtggcaccggcggcatcggcggccgggtcgcccgccgcctcgccgaacagggcgccgcacacctggtgctgaccagccggcgcggcgccgacgccccgggcgccgccgaactccgcgccgaactggagcagctgggcgtccgggtgaccatcgccgcctgcgacgccgccgaccgggaggcgctcgccgcgctcctggccgagctgcccgaggacgcgccgctcaccgcggtcttccactccgccggtgtggcccacgacgacgcccccgtcgccgacctgacgctcggtcagctcgacgcgctcatgcgcgccaagctgaccgccgcccggcatctgcacgagctgaccgccgacctcgacctggacgcgttcgtgctgttctcgtccggcgccgcggtctggggcagcggtggccagcccggctacgccgccgccaacgcctacctcgacgccctcgccgagcaccgcaggtccctcggcctgaccgcctcgtcggtggcctggggcacctggggcgaggtcggcatggcgaccgaccccgaggtccacgaccggctcgtccgccagggcgtgctggccatggaaccggaacacgccctgggcgccctcgaccagatgctcgagaacgacgacaccgcggccgccatcaccctgatggactgggagatgttcgcgcccgccttcaccgcgaaccggcccagcgccctgctgagcaccgtcccggaggccgtctcggcgctcagcgacgagggcccggccgacggtggcgccgacgcggccgtgccgccgctgcgcgcccgcctggacgggctgcccaccgccgagcgcgcccgtgccctcgtcgaggccgtccgcgccgaggcgtccgcgaccctcggccacgacagccccgacgcggtgcccgcctcgcgcgccttccgcgacgtcggcttcgactccgtcaccgccgtcgaactgcgcaaccggctccgcgccgccctcgggctgcccctgccggccgccctggtcttcgactacccgacccccacggccctcgcccagcacatcggttcgctgctctacggcaccgcacccgatgaggcggacaccggccgcgccgacgaccccgatgcccgtatccgcgaggccctcgccaccgtcccgatcggacggctgcgcaaggcgggcctcctcgacatggttctgaaactcgccgagtccgacgacaccgaggacagcgccaccgcgacggacgacgcggcggacgccctcgacctcgacgacatggacgccgaagccctgctgcggctggccgccgagaactcggcgaactgaaagagagctggaacacgatgagcacgtccaccaacaagtacgtcgaggcgctgcggtcgtccctgaaggagatcgagcggctgcgcaagcagaacgagcagctggtcgcgtcggcggcggagccgatcgcggtcgtcggcatcggctgccgtttccccggcggcgtcgtctcccccgaggacctgtgggagctcgtcgccgagggccgcgacgtcatcggtccgttcccgaccgaccgcggctgggacctggaccggctggcgggcggcggcgagggcagcagcctcgcccaggtcggcggcttcctcgacgacgccgccggcttcgaccccgggttcttcggcatctcgccccgcgaggccgtcgccatggacccgcagcagcgcatcctcctggagatcgcctgggaggcactggagcgggccggcatcgacccctccgcgctgcggggcagccgcacgggcgtgttcatgggcaccacgggacaggactacggagaggtcatcaaggcgtccgacgaggacgccgaggtgtactccaccaccggtcacgccgccagcgtgatctccggccggctctcctacttcctgggcgccgagggcccggccgtcaccgtcgacaccggctgctcctcctcgctggtctccctgcacctggccgcgcaggcgctgcgcggcggcgagtgctccctggccctggcgggcggcgcgtccgtgatggcgacgccgcacccgttcatcgcgttcacctcgcagagcggactggccgccgacggccgctgcaagccgttctccgacgacgccgacggcaccggctgggccgagggcgccggcctgttcatcctgatgcggctgtccgacgcccagcgcgagggccgcaaggtcctcgcggtactgcgcggctccgccctcaaccaggacggcgcctccaacggcctgaccgcgcccaacggcccctcccaacagcgggtcatccggcaggcactggcccacgccggtctgcaggcgagcgatgtcgacgccgtcgaggcgcatgggaccggcacgccgctcggtgacccgatcgaggcacaggccctgctggccacctacggccagaaccgcgagcggccgctgctgctcggctccgtcaagtcgaacatcggccacgcccaggccgcctccggcgccgccggcgtgatcaagatgatcatggcgatccagcacggcgtgctgccccgcagcctgcacgccgagcgcccgaccagcaatgtcgactggaccgcaggcgccgtcgaactcctcgaccgggccgtcgactggcccgagaccggccgcgcccgccgggccggtgtctcctccttcggcatcagcggcaccaacgcccacctcatcctggagcaggcccccaccgcgcccgagcgccccgccccggaccccgcggcccggcccgcgctcgtcccctggaccctgtccggccgcaccgccgccgcgctcgacgcccagcgcgagcgcctcgccgcccacctcgagagccaccccggcgccgacccgctcgacatcggcttcgcgctcgccgacggccgcgccaccttcgagcaccgggccgtgctgctctgcgccggcaccgacgcctcggccgccaccgagatcgcccacggctccgtcgacgacggcgcctcggccgtcctgttctccggacagggctcccagcgcctcggcatgggccgcgaactgcacgcccgcttcccggtgttcgccgaggccttcgacgagatcgccgccctgctcgaccgccacaccgaccgcccgctgcgcgaggtcgtctggggctccgacgccgaactcctcaacgagaccggctggacccagcccgcgctgttcgccgtcgaggtcgccctctaccgcctcgtctcctccctgggcgtcacccccgactacgtcggcggccactccatcggtgagatcgccgccgcgcatgtcgccggggtgctgtcgctggaggacgcgtgcgcgctggtcgccgcccgtgcccgcctgatgcaggagctgccgcggggcggtgcgatgctcgcggtgcgcgccaccgaggaggaggtcgccccgcatctgacggagcgggtctccatcgcgtcggtcaacggcccgtcctccgtggtcgtcgccggtgacgaggacgaagtcgccgccatcgccgcgcacttcgaggcgcaggaccgcaagaccacccggctgcgggtcagccatgccttccactcgccgctcatggacccgatgctggaggagttccgccgcgtcgcccggggcctcacctaccacgagccgcgcatcccggtcgtctccaacctgaccggcgccatcgccgaccccgccgacctgtgcaccgccgactactgggtccgccacgtccgcgaggccgtccgcttcgccgacggcgtcaccaccctcgccggccgcggcgtcaccaccctcctcgaagtcggccccgacggcgtgctgtccgcgatggcgcaggagtcactgcccgagggcaccaccggcgtcccgctgctgcgcaaggaccgccccgaggaggagtccgccctcaccggactcgcccgggtgcacgtccgcggcctcaccgtccgctgggcccgcctcttcgacagcgccggcgccgggcgcgccgacctgcccacctaccccttccagaaccagcgggtctggccggccgcgggccgctcagccaggacgtcaccggccgccggactgggctccgccgaccacccgctgctcggcgccgccgtcgaactcgccgacggcgaagggttcctgttcaccagcaggctctccgtccactcccacccctggctcgccgaccacggcgtccaggccgcgccctgcgtgcccggcaccgccttcgtcgaactcgccatccgcgcgggcgacgaggcgggctgcgaccgcgtcgaggaactcaccctcgccgccccgctggtgctccccgagcgcggcgccgtccagcttcaggtccgggtcggcgcccccgaccccgcgggccgccgcacgctcggcatcttctcccgcgtcgaggacggcttcgacctcccctggtcgcagcacgccaccggtgtcctcaccaccggcgccggcgccccggacgccgccttcgacgccaccgtgtggccgcccgcgggcgccgaacaggtcgacctcgacggctgctatgagcgcctggccggactcggcttcgactacgggcccgccttccgcgccctgcgcgccgcctggcgccgcggcaccgaggtgttcgccgaggtcgccctctccgagaacgccggcaccgaccccgccgacttcggcctgcaccccgcgctcctggacgccgcccagcacgccgccgcctacaacgacctcggcgccatcagccggggcggcctgccgttcgcctgggagggcgtgcacctcgccgcgtccggcgccaccaccgtccgcgcccggatcacctccgtgggcgaggacaccgtctccatcgccgtgtacgacaccgcgggcggcaccgtcctctccgtggactccctggtctcccgcgccgtccccaaggacatccccggcaccgcccgggccgtccaccgcgactccctcttccaggtcgactggaccccgctgcggagccagtccgggcagctcccggagaccgtcgccctcgtcggcgccgacccggccggactcgccgacacgctgcgcaccggcggcgtccaggtcaccgtccaccccgacctggccgcactggccgcggaccccgccccggtgcccgggaccgtgctcaccgccctgcccggcgaccaggacaccccggccgccgacgccgcccacaccaccgcagccgccgtactcggcctggcacagcactggctgaccgaggaccggttcgccgacgcccggctcgtcctcgtcacccgcgacgccaccgcgggcggtgacctggcggccgccgcggccggcggtctgatccgcaccgcccgcaccgagaaccccggccgcttcgtcctcctcgacctcgccaccggcacccccgccgcgcccgcggccctggtggcggccctggccgccgcgtacgacgaacccgacctcgccgtacgcggctccgacgtactgaccgcccggctcgcccgggtgcccgcacccgacgccggcgccgactggaacccggacggcaccgtcctgatcaccggcggcaccggcggactcggcggcgtcctcgcccgccacctcgtcaccgaacgcggcgtccgccggctgctgctcgccagccgccgcggcccggccgccgacggcgcccccgccctcgtcgacgaactcaccgcactcggcgcctcggtcgacgtcgccgcctgcgacgtcaccgaccgggaggcgctcgccgccctcctcgcccgggtgcccgccgcgcacccgctgagcgcggtcgtgcacaccgccggcgtggtcgacgacggcgtcatcggctccctcaccgccgagcgcctcgacaccgtgctgcggcccaaggccgacgccgcctggcatctgcacgagctcacccgggacctggacctggacgcgttcgtgctcttctcgtccgtcgccgccaccctcggcagccccggacagggcaactacgcggccggcaacgccttcctggacgccctcgccgcccaccgccacgccctcggcctgcccgccacctccctcgcctggggcccctggacccagagcgtcggcatgaccggcaccctctccgacatcgacgtcgagcgcatcgcccgctccggcatgcccccgctcaccgtcgagcagggcgtcgccctcttcgacgcggccctcgccaccggcggccccgccctcctgccggtccgcctcgacctggccgtgctgcgcacccagggcgagatcgcgccgctgctgcgcggactgatccgcaccaccgtgcgccgcgccgccgcccaggtgtcggtgaccgccgacggcctcgcccagcaactggccggactggacgccgccgcccgccgcgaggccctcctcggcctggtccgcacccagatcgcccaggtcctcggccacgccgacgccggccaggtggaggcggcccgccagttccaggacctcggcttcgactcgctcaccgcggtcgaactgcgcaacggcctcaactccgccaccggtctgcgactgcccgcgaccatggtgttcgactacccgaccccgaacgcgctcgccgaccacctgagcgacgaactcctcggcaccggcgacaacatacccctgcccacccccgcgggcgccctcccggccaccaccgacgacccgatcgtcatcgtcggcatggcctgccgctacccgggcggcgtcacctcgcccgaggacctgtggcgcctggtcagcgagggcgccgacgccaccggaccgttccccgtcaaccgcggctgggacgtcgacaacctctacgacccggaccccgaccgccccgggcacacctatgtgcgcgccggcgggttcctgcacgacgccggctccttcgacgccgacttcttcggcatgagcccgcgcgaggcgctctccaccgactcccagcagcgcctgctgctcgaactgtcctgggaggccgtcgagcgggccggcatcgacccgcacagcctgcgcggcagccgcaccggcgtcttcgccggcgtcatgtacaacgactacggcaccatcctcaccggcgaggagtacgaggccttccgcggcaacggcagcgcccccagcgtcgcctccggccgcgtctcctacaccctgggcctggagggccccgcggtcaccgtcgacaccgcctgctcctcctccctcgtcggcatgcacctcgccgcccaggcgctgcgcacgggggagtgcacgctcgccctggccggcggtgtcaccgtgatgtcgacccccagcaccttcgtcgacttctcccgccagcggggcctggccgccgacggccgctccaaggcgttcgccgaggccgccgacggcgtcgcctggtccgagggcgtcggcatgctcctcctggagcgccggtccgacgcgatccgcaacgggcacgagatcctcgccgtgctgcgcggctccgccgtcaaccaggacggcgcctccaacggcctcaccgcccccaacggcccctcccaacagcgggtcatccgccaggccctcgccagcggcggactgtcccccgaggacgtcgacgtcgtcgaggcgcacggcaccggcaccaccctgggcgaccccatcgaggcccaggcgctgatcgccacctacggccgtgaccgtgacccggagcggcccctgtggctcggctccgtgaagtccaacatcggccacacccaggccgccgcgggcgtcgcaggcgtcatcaagatgatcatgtcgatgcggcacgggctgctcccgcagaccctgcatgtggacgccccgtcctcgcacgtcgactgggacgccggagccgtcgaactgctcaccgagcagaccgagtggcccgagaacgaccgggtgcgccgcgccgccgtctcctccttcggcatcagcggcaccaacgcccacgtcatcatcgagcagccggccgccggcccggccccggccgccccgagcggccccgcggaggaacttccggccgccccctggccgctgtccgcgaagacccccgaggccctgcgcgaccaggccgcccgactgctcgcccatgtcgaggcccaccccgagacgcgccccgccgacatcgcgtactcgctgctcaccagccgctccgtcttcgaccaccgcgccgccgtcctcggcgccgaccgcaccgaggcgctccgcgccctcgcggcccttgccgcaggtaccgacgacagcgcgctgctgaccggcaccgcccgcggcggccgcaccgcgttcctgttctccggccagggctcccagcgcctcggcatgggccgtgagctgtacggcagggtccccgtgttcaccgaggccctggacgccgtactcgccgtcctggacggcgagttggagggttcgctgcgggaggtgatgtggggtgaggacgccgacctgctgaacgagaccggatggacccagcccgcactgttcgccgtcgaggtggcgctctaccgactggtggagtcctggggtgtgaagccggacttcgtggccggccactccatcggtgagatcgccgccgcgcacatcgccggggtgctcaccctggcggacgcctgccgtctcgtcgccgcccgcgcggccctgatgcaggcgctgcccgagggcggcgccatgatcgccgtcgaggccaccgaggacgaggtcctcccgctcctcaccggcgacgtctccatcgccgccgtcaacggaccgtcgtccgtggtcgtctccggcgccgaggccgccgccctcgcggtcgccgggcacttcaccgagcagggacggcgcaccagccggctgcgcgtcagccacgccttccactcgccgctgatggacccgatgctggacgagttccggtcggtggccgaggggctctcgtactccgccccggcgatcccggtcgtctccaacctcaccggcaccctcgccgaccccgccgacctgtgctccgccgactactgggtgcgtcatgtccgcgacgccgtccggttcgccgactgcgtcggcaccctcggcgaacacggtgtcaccaccttcgtcgaactcggccccgacggtgtgctgtccgccatggcgcagcagtccgtcaccgagggcgccgtcaccgtgcccgtgctccgcaaggaccggagcgaggagcaggccctcgtcaccgccctgtgccggctgtacgtcctcggcatcgacgccgactggacctccgcgctcaccgtcggcggggccgcccgcgtcgacctgcccacctacgccttccagcaccgctggttctggcccgccgcccgagcggcacatccgcacgacatgcgcgccgccggactcggctccgcatcccaccccctgctcggcgccgccgtggaactccccggcggggaaggcgtgctgctcaccggtcgcctctccctccagtcccacccctggctcgccgaccacgccgtgcacgggaccgtcctgctgcccggcaccgccctcctcgacctcgcgatccgcgcgggcgacgaggccggctgcggtcacctcgaggacctcaccctcgccgcgccgctgaccctgccgcaggacggcgccgtgctgctccaggtccgggtgggagccgccgacgacgccggccgacgcaccgtcaccgtgcacgcccgccccgaggacgccaccgacggcgtctggaccctgcacgccaccggcaccctgtccgacaccgccgccaccggcacggccctcgacaccaccgtctggccgccggccggcgctcaggccctcgacaccgagggctgctaccagcgcttcgccgcccacggcttcgcctacggtccgctcttccagggcctcaccgccgcctggaaggccggcgaggcgctgtacgccgagatcgcgctgcccgaggagggccacgacgacgcggccggcttcgccgtgcaccccgcactgctcgacgccgctctgcaccccgcgctgctcaccgacgacgacggtggcctgccgttctcctgggagaacgtcagtctgcacgcctccggcgccaccgcgctgcgcgtccgcctcgccccggcgggtgcgaacgcgctgtccgtgaccgccgccgacccggccggccggcccgtggtctccatcgcccggctgctcgcccgcccggtcgaggccgaccagctgaccggcgacgccggacacctgcgggacgcgctcttccggctggagtggaccccggtcccgctgcccgacggcacgcccaccgagagcctcgccctgctcggcccggacaccgacgacctggccgaggccctcggagagggcgccgtccggtatgccacccttgccgatctgctcgcggccgacgcgccggtccccggtgccgtactggttccgctcacggccggctccgacaccgatgacacgccgggcgccgtgcacactcgcaccgccgccgccctgaccctccttcaggagtggctggcggaagaccggttcaacggttctcgtctggtcctcgtcacccgcggcgccgtcgccaccgacgacgcgccggtgaccgacctcgccgctgccgcgctggtgggcctggtgcgctctgcgcagaccgagaatcccggtgtcttcgggttcatggatctggacgccgacggcgcctccgactccgcccttacccgtgccctcggcctcgatgagccgcagctcgcgctgcgcggaggccgtgccctcgccgcccgtctgacccgcgccaccgccgactccaccgcccgcgtcgcctggtcgtccgaggggacggtgctggtgacgggtggtaccggtggtctgggtggtctggtggcccgtcatctggtcatggagcgtggggtgcggcgtctgctgctgaccagccgctccggtctcggtgccgcgggcgccggtgacctggtggcggagctggaggggctgggtgccgaggtgtcggtggcggcctgcgacgtcgccgaccgggacgcggtgaccgcgctggtcgcggcggttccggcggagcacccgctggtcgcggtcgtccacacggccggtgtactcgacgacggtgtgctcgactcgctcaccgaggagcgccttgccacggtgctgcggcccaaggtggacggcgcctggaacctccacgaggcgacgcgtcacctggacctggacgcgttcgtggtgttctcctccgtcgccggtgtcttcggcggcgcgggtcaggccaactacgcggccgccaacgccttcctggacgccctcaccgcccaccgccacgccctcggactgcccgccgtctccctcgcctggggcccctggcagcagggcgcgggcatgacggccgggctcgacgagcgggacatccgccgggccgcagagtccgggatgccgctgatcaccgtcgagcagggcctggccctcttcgacgccgccctcaccaccgatgaggccgctctcgtcaccagccgtctggacctcgccgccttccgcggccggcccgagatcccggccctgctgcgcggactgatccgcacccccgtgcgccgcgccgccgcgaccggggccgccgacggtgccgacgccggactcgtccagcggctcgccgggctcgaccgcgagcagcgccacgaggtcctgctcgacctcgtccgtacgagcgccgccctggtcctcggccacgccgccggctccgacatcgacgccgaccgcgccttccgcgacctcggcttcgactccctcaccgcggtcgaactccgcaaccgcctgcgcaccgccaccggtctgcgcctgtcggccaccatggtcttcgaccatcccaccctgtccgcgctcgccgaccacctgctcaccgagctgttcggcgccgaggccgagaccctcacccccgtcacgctgctgccgccgaccgacgacgacccgatcgtgatcgtcggcatggcctgccgcttcccgggcgacgtcacctcgcccgaggacctgtggcgcctggtcagcgagggcaccgacgccatctccgacttcccggccaaccgcggctgggacgtggacagcctctaccacccggaccccgagcactccggtacctcgtacacccgctccggcggattcctccacgacgcgggcgacttcgaccccgacttcttcgggatgagcccccgcgaggcgctcgcgacggactcccagcagcgactgctgctcgagacctcctgggaggccatcgagcgggccggcatcgacccgctcagcctccgcgacagccgcaccggtgtcttcgccggtgtcatgtaccacgactacggcagcctgctgggcggcaaggagttcgagggcttccagggccagggcagcgcgggcagcgtcgcctccggccgggtctcgtacaccttcggcttcgaaggccccgcggtcaccgtcgacaccgcctgctcgtcctccctggtcgccctgcactgggccgcccaggcactgcggtcgggcgagtgctcgctcgccctggccggcggcgtgaccgtgatgtccacccccagcaccttcatcgagttctcccgccagcagggtctctccgccgacggccgctccaaggcgttcgccgagtcggcggacggtgtcggctggtccgagggcgtcggcatgctcctcctggagcggcagtccgacgcgatccgcaacggccatgagatcctcgcggtcgtccgcggctccgcggtcaaccaggacggcgcctccaacggcctcacggcccccaacggcccgtcccagcagcgggtgatccgccaggccctggccagcggcggactctcgaccggcgacgtggacgccgtcgaggcgcacggcacgggcacgaccctcggcgaccccatcgaggcccaggcgctcatcgccacctacggccagaaccgcaccgacgagcaaccgctgtggctcggttcgctgaagtcgaacatcggtcacacccaggccgcggccggtgtcgccggtgtcatcaagatggtcatgtcgatgcggcacggtgtgctcccgtgcaccctgcacgtcgacgccccgtcgtcccacgtcgactggaccgaaggggccgtcgaactgctcaccgagcagaccgagtggcccgagacggaccgggtgcgccgcgccgctgtctcctccttcggcatcagcggtaccaacgcccatgtcatcctggagcagccggtcgtcgcaccggcaccggcgacggccgatgtggacggcgccgaacccgccgtcgtcccgctgatcctctccggcaagtcacccgaggcgctgcgcgatcaggccgcccgcctgctcgacaccgtccgcgagcacacggcgctgcgcccgctcgacctgggccactcgctggcgaccagccgttccgcgttcgaccatcgggccgtcgtcctggccaccggccgggaggacgccctccgcgccctgaccgccctcgccgacgacgaggcgaactccgccgccgtcaccggccgcacccggtcgggccgtcgtgcggcgttgttctcgggtcagggttcgcagcggctcgggatgggtcgtgagctgtatggccgtttcccggtgttcgcggaggccctggatgccgtactcgctgtcctggacggcgagttggagggttctctgcgggaggtgatgtggggtgaggatgccggtctgctgaatgagaccgggtggactcagcccgccctcttcgcggtcgaggtcgctctctatcggctggtggagtcgtggggtgtgaggccggacttcgtggccggtcactccatcggtgagatcgctgccgcgcacattgccggggtgttctcgctggaggatgcggcgcgtctggtggctgctcgtggtcgtctgatgcaggcgctcccggccggcggcgcgatggtggccgtccaggccaccgaggacgaggtcatcccgtacctgtccgacgaggtgtcgatcgccgcactcaacggacccgcgtccgtggtggtgtcgggggccgaaggtgccgtgctggaggttgcggcgcggttcgaggcggagggccgtaaggcgacgatgctgcgcgtgtcgcacgccttccactcgccgctgatggacctgatgctggacgagttccgggcggtggccgagacgctgtcgttcgcggccccggtgatcccggtggtgtcgaacctgacgggttcgctggccacggcggaggagctgtgctcgcccgagtactgggtccgtcacgtccgtgaggcggtccgtttcgccgacggggtgagcgcccttgaggcgcagggcgtgaccacgttcctggagctgggccccgacggagtcctctccgcgatggcccaggagtcgctgaccggcgacgaggccgtcaccgtcgcgctgctgcgcaaggatcgcgacgaggagacggccgccctgaccgccctcgccgccctgcacacggcgggcgtgcggctcgactggaacgccttcttcgccggcaccggcgccacccgcgtcgacctgccgacctatgcgttccagcacaccacctactggcccagcggcacccactccgccgcggacgccgccgccgtaggcctcaccgccgccggacacccgctgctcaacggctcggtgcagctcgccgagggcgagggcgtgctgttcaccggccgtctctccctccagtcccacccctggctcgccgaccacgccgtgcacgggaccgtcctgctgcccggcaccgccctcctcgacctcgccttccgcgcgggcgacgaggttggctgcgaccgcgtcgaggaactcaccctcgccgccccgctggtgctgccggagcgcggcgccgtccagacccaggtcagggtcggcgtcgccgacgacaccggccgacgcaccgtcaccgtccactcgcgccccgaggacgcgacggacgtgtcgtggacgcagcacgccaccggcaccctgatgccgggcggcaccgcccgcgacaccggcttcgacgccaccgtgtggccgccgaccggcgccgaggctctcgacaccgagggctgctacgagcgtttcaccgccctcggcttcggctacggcccggtcttccaggggctgaaggccgcctggcgcagcggagacgtcctctacgcggaggtcgtcctccaggaggccggcgccgccgacgacgaggccggcttcggtctgcaccccgcgctgctcgacgccgccctgcacgcctcgctcgtcgcccacgagggcgaggagggcaacggcgggcttccgttcgcctgggagggcgcggtgctgcacgccaccggcgccaccgcgctgcgggtccggctgaccccgaccgacgaggcccggcagtccgtggcgatcgcggtctgcgacaccgccggtcagccggtcgcctccatcgaccggctcctggtcagggccgtcaccggcgaccagctgaacgaggccgccggcatcgcccgcgacgccctgttcgccctcggctggaccccgctcccgcaggacggcgacatcccggcagccgtcgcgctcgtcggcccggacaccaccgggctcgccgcggacctgaccgcagccggcgccgccgtcacggcccaccaggacctcgccgccctcgcggcggacggcaccgacatccccggcaccgtcgtcgtcaccgtcgacggcaccgccgcaccggtcgccgaggccgcgcacaccgtggccgccgaggccctcgctctcgcccagcggtggaccgccgaggaccggttcgccggggcccggctggtgttcgcgacctgcggcgccgtcgaggcgggcggctcccaggtcaccgacgtcgccgccgccgcggtctggggcctggtgcgctccgcccagtccgagagcccggacacctttgtactcgtcgaccgggaggcggccgacgccgccggcgacgccgccgaacgcgggagcctgttcctggcggcgctcggcagcggcgaaccgcagctcgcgctccgcgacgacgtggttctggccggccgcctcgcccgcttcgatgccgccggcgccatgacgctgccggccgagcgggcctggcacctcgacagcaccacgcccggcagcatcaacggcctcgccctcaccccgcacccggaggccctggagccgctcaccggacaccaggcccgtatcgaggtccgcgccgcgggcctcaacttccgcgacgtcctcaaggccctcggcatgtatcccggcgacgccgggcgatacggccacgaggccgccggcgtggtcgtcgaggtcggcccggacgtcacgcacatcgcccccggtgaccgcgtcatgggcatggtctccggtagcttcgcctcgttcgccgtgaccgacgcccgccgtctcacccacctcccggagcagtgcacctgggagatcggtgcctccctgccgctggtgttcctcaccgcctaccacgcgctgaaggaactcggcgggctgaccgcgggggagaaggtcctcatccacgccggtgccggtggtgtcggcatggcggcgatccagatcgcccgccacttcggcgcggaggtcttcgcgacggccagcgaggccaagatggacgtgctgcgctccctcggagtcgccgacgaccacatcgcctcctcccgcaccctcgacttcgaggcggccttcgccgccgtcgcgggcgagaacggtctcgacgtcgtactgaactccctggccggggagttcgtcgacgcctcgatgcggctgctcggcacgggcggccggttcctggagatgggcaagaccgacatccgcgaggacgactcggtcccggacggcatcacctatcagtccttcgacctcgcctttgtggaccccgaggtgatcggcgccatgggccgcgagctgacggagctgttcgccgccggagacctccacccgctcccggtccgtgcctgggacatccggcacgccacggacgccttccgccatatgagcatggcccggcacatcggcaagatcgtgctgaccgtgccccgcgcctggtcgtccgaggggacggtgctggtgacgggtggtaccggtggtctgggtggtctggtggcccgtcatctggtcatggagcgtggggtacggcgtctgctgctgaccagccgctccggtctggacgccgcgggcgcccgtgagctggtggcggaactggagaacctgggtgccgaggtctcggtggcggcctgtgacgtcgccgaccgggacgcggtggacacgctgatcgcgggcatcccggcggagcatccgctccgcgcggtggtgcacacggccggtgtgctggacgacggtgtgctcggctccctcaccgaggagcgccttgccacggtgctgcggcccaaggtggacggcgcctggaacctccatgaggcgacgcgccgtctggacctggacgcgttcgtggtgttctcgtccgtcgccggtgtcttcggcggcgcgggtcaggccaactacgcggccggcaacgccttcctggacgcactcaccgcccaccgccagtccctcggactgccgggcctctcgctcgcctggggcgcctgggcgcagggcgcgggcatgacggccgggctcgacgagcgggacatccgccgggccgcagagtccgggatgccgctgatcacggtcgagcagggcctcgccctcctcgacaccgcactcaccaccggcagcgccgccctcgtcccggtccgcctggacctcgccgtgctgcgcgcccggggaaccgtcgcaccgctgatgcgcggtctcgtccgcgcccccgcccggcgcgcggcggccaccgccgccacgggcgacaccgcgctcgtcgaccggctcacccggctgcagcgcaccgaacggcgcgacgccctgctcaccctggtccgcgaacaggccgcgctggtgctcggccactccggcggcggtggcatcgacccgtcccgcgccttccgcgacctcggcttcgactcgctcaccgcggtcgagctccgcaaccggctcggtgccgcgaccggtgtccggctgtccgccactgcggtcttcgactacccgaccgtcgtcgccctcgtcgaccacctgctcaccgaactcctgggccccgacgccgagtcggacgccgaggaggccgcgccggccgaggcgctcctggacgacgacccgatcgtgatcgtcggcatggcctgccgcttccccggcggaatcacctcacccgaggacctgtggcgactgctcggcgaaggctccgacgtcatctcggacttccccgtcaaccgcggctgggacgtcgactcgctgtacgacccggacccggcccacaccggcacgtcgtacacccgctccggcggcttcctgcacaacgccgcggacttcgacgccgacttcttcggcatgagcccgcgtgaggcgatggccaccgactcgcagcagcgcctgctgctcgagtcgacatgggaggccatcgagcgggccggcatcgaccccgtcaccctgcggggcagccgcaccggcgtcttcgccggcgtcatgtacagcggctacggcaccgcgctcgacggcgacgagttcgagggcttccagggccagggcagcgcgctgagcgtggcctccggccgggtctcgtacacgttcggcttcgagggcccggcgatgaccatcgacacggcgtgctcgtcggcgctggtggcgatgcacctcgcggcgcaggcactgcgctccggcgagtgctcgctcgccctggccggcggtgtcaccgtcatgtcgatcccggacaccttcgtcgagttctcccgccagcggggtctgtcgcccgacggacggtgcaagccgttctccgagtcggcggacggtgtcggctggtccgagggcgtcggcatgctcctcctggagcggcagtccgacgcgatccgcaacggccaccgcgtcctcgccgtggtccgtggttccgccgtcaaccaggacggcgcctccaacggcctgacggcccccaacggcccgtcccagcagcgggtgatccgccaggccctggccagcggcggactctcgaccggcgacgtggacgccgtcgaggcacacggcaccggtacgacgcttggtgacccgatcgaggctcaggccctgctcgccacctacgggcagaaccgcccggaggaccggccgctgctgctcggctccgtgaagtccaacatcggtcacacccaggccgcggccggcgccgccggcgtcatcaagatggtcatggcgatgcagcacggtgtgctgccccgcagcctcaacatctccgagccgtcctcgcacgtcgactggagcgccggtgccgtcgaactgctcaccgagcagaccgagtggcccgagacggaccgcgtgcgccgtgccggtgtgtcgtccttcggcatcagcggtaccaacgcccacgtcatcctggagcagcccgcgaaggtgatccagggcacggtggtggctgcctcggcggcaccggggccggatgtggtggagcccgcggtggttccgtgggtgctgtccggcaagacgccggaggcgctgcgcgaccaggccgcccgcctgctcgccacggtcaccgaggagccggcaccgcgtccggtcgacgtggcgctctccctcgcgacccagcgttcgcagttcgcccaccgcggtgtggtgctggccggtgagcacgaggagacggtgcgcgccctggccgccctcgccgacggcgagccccactcctccgtcaccaccggcaccgtgtcgtcgggccgtcgtgcggcgttgttctcgggtcagggttcgcagcggctcggtatgggtcgtgagctgtacggccgtttcccggtgttcgcggaggctctggatgctgttctcgctgttcttgacggtgagttgggtggttcgctgcgggaggtgatgtggggtgaggatgccggtctgctgaatgagaccgggtggactcagcccgcgctgttcgcggttgaggttgctctctatcggctggtggagtcgtggggtgtgaggccggacttcgtggccggtcactccatcggtgagatcgctgccgcgcacattgccggggtgttctcgctggaggatgcggcgcgtctggtggccgctcgtggtcgtctgatgcaggcgctcccggccggcggcgcgatggttgccgtccaggccacggaagacgaggtcgtcccgcatctgtccgacggggtgtcgatcgccgcgctcaacgggcctacgtccgtggtggtgtcgggtgtcgaggacgcggtgttggaggtggccgcgcggttcgaggcggagggtcgtaagaccacccggctccgcgtgtcgcacgccttccactcgccgctgatggacccgatgctggacgagttccgggcggtggccgaggggctgtcgttcgcggccccggtgatcccggtggtgtcgaacctgacgggttcgctggccacggcggaggagctgtgctcgcccgagtactgggtgcgccatgtccgtgaggcggtccgcttcgccgacggggtggccgcccttgaggcgcagggcgtgaccacgttcctggagctgggcccggacggagttctctccgcgatggcccaggagtcgctgaccggcgacgagaccgtcacgatcccgctgctgcgcaaggaccgcgacgaggagacggcagccctgaccgccctcggccggctgcacgccaccggcacccgagtcgactgggccgggttcctggccccggccggcgcccgcaccgtcgacctgccgacgtacgccttccagcaccggccgttctggcccagcggcaagcgcggcgcgacggacgtcaactccgtcggcatcaccgcggcggcccacccgctgctcaacgcggtcgtcgaactcgccgagggcgagggcctggtcttcaccgggcgcctgtcgctgaactcgcacacctggctcgccgaccacgccgtcatgggcaccgtcctgctgcccggcaccgccctgctggagctggcgctccgcgcgggcgacgaggtcggctgcgaccacgtcgaggaactcaccctggccgccccgctggtcctcacggagcagggcgcggtccgtacccaggtgcgggtgggcgccgaggacgccgccggacgccggaccgtcacgatccactcgcgcccggagcggtccaccgacgccccctggacccagcacgccaccggcgtcctcgccaccgggctcccgacggccgccgcgccgttcgacgccacggtgtggccgccggcggacgccgagcccgtcgacctgaccggcttctacgaggcacgcgaggccgagggcttcgcctacggtcaggcgttccagggcctgaaggccgcctggcgccgcggcgacgaggtgttcgccgagatcacgctgccggacggcacacagaaggaagccgagagcttcggcctccacccggcgctgctggacgccggtctgcacgccgcctggctcgcggccccgaccggtgacggcacgaccggcagcgtcccgttctcctggaacggcgtctcgctggtcgcctccggcgcctcgtccgtccgcgtccggctcggccgtgacaccgacggcacgctgaccctggccgtcgccgacaccaccggcgcgcccgtggccgccgtccaggcgctcaccatgcgcgccgtgtccgcggaggcgctcagctccgccgcggcactcgtccgcgactccctgttccgcctcgactggaccccggtcaccccggccgggtccgcggagccgacggcggcactcgtcggcgaggatcccttcgggctcgtccggagcctgcgggaggccggcaccgagatcggccggcgcgcgtccctgacggacgacgccgagctgcccgacctgctgctgctgcccgtcaccgccgaggagcacgaggaggcggccgacacgccggccgtcacccacgcactcacccggcaggtgctggaactggtgcgggccaggctggaggacgagcgtcacgccgccacgaggttcgtcttcgtgacccggggcgcgaccaccggccacgacctggccgccgccgcggtctggggcctggtgcgctccgcccagtccgagaacccgggctgcttcggcctgatcgacctcgaccccgagaccacccgtccgctcccgctcaccgccctcctgggcgacgaacctcagctccgcctccaggacgacgacctcagggccgcccgtctggtccgccgccctgcccccgccactgacgcggtgtccgcgttcggtggggagggtgcggtgttggtgacgggtggtaccggtggtctgggtgcggtgctggcgcgtcatctggtggccgagcacggggtgcgtgagctggttctggtcagtcgtcgtggtggtgcggcggcgggtgcggccgagctggtcgcggagctggcggagtcgggtgcgcgggccacggtggtggcctgtgatgtgacggaccgtgcggcggtggccgagctggtggccgcgcatccggtgtcggcggtggtgcactccgccggtgtcctggacgacggcctgatcgcctccctcaccccggaacgactctccggtgtgctgcgtcccaaggtggacgcggcctggaacctgcatgaggccacccgtgacctggacctggacgcattcgtggtgttctcgtccgtcgccggtgtcttcggcaacgcgggtcaggccaactacgcggccggcaacgccttcctggacgccctgatggaacaccgccgtgcggccggcctgccgggtctgtcgctggcctggggcccctgggagcagaccggcaccggcggtatgaccggcacgctctccgacgcggagctcgagcggctcgcgtcctccggagtgcccccgctcgccgtcgagcagggtctggccctcttcgacgccgcgctgtccaccgaggacgccgcgctcgtgcccgtccggctcgacctgcccgccctgcgcggccagggtgaggtgccgccgctgctccgttcgctgatccgggcccgctcgcgccgtgcggcggtcgcgggctcggcgacggcgaccggtctgcgcgaccggctggccgtcctggacgcggtcgagcgacgggaagtgctcgtggacctcgtccgcggccaggtcgccctggtgctcggccacgccgacgccacggccgtccaccccacgcgtgccttccgggacctcggcttcgactccctcacctcggtcgaactgcgcaaccgcctgaacgccgtcaccggactgcgtctgcccgccacgatggtgttcgactacccgaccgtcgaggtcctggtcgacttcatcctggacgagcttctcggcaccgaggtcgaggcggaggtcgttcagcgcggcacggcggccgtggcggacgacccgatcgtgatcgtcggcatggcctgccgctatccgggcggcgtcgcctcgcccgaggacctgtggcgactggtcaccgagggcacggacgccgtctccgccttcccggtcaaccgtggctgggacgtggagaacctctaccacccggaccccgacaacatcggtacgtcctacacccgttccggtggcttcctgcacgaggcgggggagttcgaccccggcttcttcggcatgagcccccgcgaggcgctctccaccgactcccagcagcgtctgctgctcgagacctcctgggaagcgatcgagcgggccggtatcgacccggtcggtctgcgcggcagtgcgaccggtgtgttcgccggtgtgatgtacagcgactacagcgcgatgctggcgagccccgagttcgagggcttccagggcagcggcagttccccgagcgtggcttccggccgggtctcgtacaccttcggcttcgagggtccggcagtcacggtggacaccgcgtgctcgtcgtcgctggtggcgatgcactgggcgatgcaggccctgcgctcgggtgagatctcgctcgccctggcaggcggtgtgaccgtgatgtcgacaccgggagtcttcgtggacttcgcgcggcagcggggtctgtcgccggacggacggtgcaaggcgttctcggactcggcggacggtgtcggctggtccgagggtgtcggcatgctggtcctggagcggcagtccgacgcgatccgcaacggccatgagatcctcgcggtcgtccgcggctccgcggtcaaccaggacggcgcctccaacggcctcacggcccccaacggcccgtcccagcagcgcgtcatccgccaggccctcgccagcggtggactctccgcccaggagatcgacgtggtcgaggcgcacggtacgggtacgacgctgggtgacccgatcgaggcgcaggcgctgcttgcgacctacggccgggaccgtgacccggagcagccgctgtggctcggctccgtgaagtcgaacatcggtcacacgcaggccgcggcgggtgtcgcgggtgtgatcaagatggtcatgtcgatgcggcacggtgtgctcccgcgcaccctgcatgtcgatgccccgtcctcgcacgtcgactggaccgaaggggccgtggaactgctcaccgagcagacggagtggcccgagacggagcatgtgcgtcgtgcgggtgtctcctccttcggtatcagcggtaccaacgcccacgtcatcctggagcagcccgcaaaggtgatccagggcaccgtgatcggtggttcgacacccgagtcgggcgtggtggagccctcggtggttccgtgggtgctgtccggcaagacgccggaggcgctgcgcagccaggcggcgaagctgctcgcctcggtcgaggcggagctcgaccgtccgctggtggacgtcggttcctccctggtcgccgcccggtcgctgttcgagcaccgtgcggtcgtcctcgccaccgacgcggacaccgcggcccgcgccctggcggccctcgcggtgggcgagcccgaccccgcggccgtatcgggcccggcccgcaccggtcgctcggcggcgctgttctcgggtcagggttcgcagcggctcggtatgggtcgtgagctgtacggccggttccccgtgtttgccgaggccctggatgccgtactcgctgtcctggacggcgagttggacggttctctgcgggaggtgatgtggggtgaggatgccggtctgctgaacgagaccgggtggactcagcccgcgctgttcgcggtcgaggtcgctctccaccgcctcgtggagtccttcggagtcactccggacttcgtggccggccactcgatcggtgagatcgctgccgcgcacattgccggggtgttctcgctggaggatgcggcgcgtctggtggccgcccgtggtcgtctgatgcaggcgctcccggccggcggcgcgatggtggccgtccaggccaccgaggacgaggtcatcccgtacctgtccgacgaggtgtcgatcgccgcactcaacggacccgcgtccgtggtggtgtcgggggccgaaggtgccgtgctggaggttgcggcgcggttcgaggcggagggccgtaaggcgacgatgctgcgcgtgtcgcacgccttccactcgccgctgatggacccgatgctggacgagttccgggcggtggccgaggggctgtcgttcgcggccccggtgatcccggtggtgtcgaacctgacgggttccctggccacggccgaggagctgtgctcgcccgagtactgggtgcgccatgtccgtgaggcggtccgcttcgccgacggggtcaccacgctcgaagaacagggcgtgaccaccttcctggagctgggtccggacggagttctctccgccatggcccaggagtcgctgaccggcgacgaggccgtcaccgtcccgctgctgcggaagaacacggccgaggaggccgcgacggtcgccgcgctcgcccgactgcacatcggcggactgcggatcgactggtccgccctgttcgccggtaccggcgcacgccgcgtggaactgccgacgtacgccttccagcaccagtggttctggcccgcggccccgctcggcggcggcgatgtccgcgccgccggtctcggctccgccgagcaccccctgctgggcgccgccgtcgaactcgccgcaggcgagggcgtgctgttcaccggccgcctcgcactgcactctcacccctggctggccgaccacaccgtcgacggcaccgtcctgctgccgggcacggccctgctggagctggcgatccgcgccggcgacgaggtcggcagcgaccaggtcgaggaactgaccatcaccgcaccgctggtcctgcccgagcggggcgccgtccaggtccaggtcgccgtcgaggcacccgatgccgccggccgccgcgtcgtgggcgtgtacgcacgccccgaaggcaccggcgacaccgcctggtcgcagcatgccgccggtgtgctggcccccgccggcagccggccggcccccgccgccttcgacaccgccgtatggccgcccgagggcgcccaggccgtcgacgccgacggatgctacgaggagttcgccgcggccggcttcggctacggaccgctcttccaggggctgcgcgcggcctggcggcgcggcgacgaatggttcgccgaggtggctctgccggagggcaccgagaacacggccgccggcttcggtctgcacccggccctgttcgactccgcgctgcacgccctactgctgaccccggccgccgacggcggcacggccggactgccgttctcctggacggacgtctcgctgcacgcctccggagccaccgtgctccgtgtccggctgacgccgaccggcgaccgtacgctgtcgctgagcgccgtggacccgtccggtacgccggtcgtctcggtgggctccctggtcacccggagcgtcgacggactcggcaccacgggaaccgaggcgctgctgcgtgacgcgttcttcggcgtcgagtggacccccgcccagggatccgaggcggcccccgcgaccgtcgcgctcctcgggaccgacggcgacccgctcgggctcgccgccgcgctcaccggcgccggcaccaccgtgcggacctacccggacgtggccgccctggtggccgccacggtcgccgcggacgggcccgtgccggaggtggtcgtgcacggcgtcgcctcgcacgacggcgaaccggcctccgacgcggcacaccggctcaccgccgacgcgctggacaccgtccagagccggctgtccgaagaccggctcgccggcgcccgcctcgtcttcgtgacgcgcggcgcggtcgacacccgggacctcggcgcggcggccgtccagggtctggtccgttcggcccagaccgagaacccgggcgccctcggcctcgtcgacctggacgggacgcccgagtccgccgccgccctcgcagcggccctcggctccgccgaaccgcagctcgcgctgcgcgacggccggccgcacgccgcccggctcgcccggctggcccgtcccgccgacaccgccgcgcccaccgcgtggcagtcggacggcacggtcctggtcaccggcggcaccggtggcctcggcgcccagttcgcccggcacctcgtcgacgcctacggggtccgcaacctgctgctggtcagcaggcgcggcggtgacgcccccggcaccacggaactcgtcgccgaactcatcgcgcatggtgcggaggtgacggtccaggcctgcgatgtcgccgacccggacgccgtgaccgccctggtcgccgggatccccgcggaacacccgctcaccgcggtcgtgcacaccgcgggcgtcctcgacgacggtgtcatcggctccctcaccggggagcggctcgcccgggtactgcgcccgaaggcggacgcggcctggaacctgcaccaggccacccgcggtctggacctggacgcgttcatcgtgttctcgtccgtggcgggagtcttcggcggcgcgggtcaggccaactacgcggcgggcaacgccttcctggacgcgctcatggagcaccgccgggcggccggcctgccgggcctgtcgctggcctggggcccctgggaccaggcgggcggcatgacggaaaccctctcggacgccgaggccgaacggctggcgcgtgccgggatgccgccgctcgcggccgagcagggcttcgccctcttcgacgcggcgctcgcggccggcaacggcaccggcaccggctctgaggacaccaccggcaccgccctggtcgtcccggtccggctcgacctggcggcgctcgccgcgcagggcgaggtgcccgcggtgctccgcggtctcgtccgcacccgcacccggcgcactgtcgcgggcggaaccgtcaccgccgacgggctcgtggcacgcctgaccgcgctcacggccgaagaacgccgggaggcgctcctcggcctggtccgcacccaggcggcgctggtcctcggacacgccgacgccgcgtcggtggacgccgcggcccagttccgggacgtcggcttcgactcgctcaccgcggtcgagctgcgcaaccggctgagcgcggccaccggactgcgcctgaccgcgacgctggtgttcgactacccgaacgcggccacgctcgcgagccatctgcacgacgaactcttcggagccgagaccgagtcggcgctcgcgctcccggtgaagcaactgcccgccacgaccgacgacccgatcgtcatcgtgggcatggcctgccgctacccgggcggggtcgcctcgcccgaggacctgtggcgactgctcctggagggcaccgacgccatcaccgagttccccgtcaatcgcggctgggacatcgagtcggtctaccacccggaccccgagcacctcggcacctcgtacacccgttccggtggcttcctgcacgaggcgggggagttcgaccccggcttcttcgggatgagcccgcgtgaggcgctggcgacggactcccagcagcgtctgctcctcgagtcgtcgtgggaggcgatcgagcgggcaggtatcgatccggtcggtctgcggggcagtgcgaccggtgtgttcgccggtgtgatgtacagcgactacggctccatcctcggcggcaaggagttcgaaggcctccagggccagggcagcgcgggcagcgtggcctccggccgggtctcctacaccttcggcttcgagggtccggcggtcaccgtcgacaccgcgtgctcctcgtccctggtggcgatgcacctcgcggcgcaggcactgcggaccggcgagtgctcgctcgccctggccggtggtgtgacggtcatctccaccccgggcaccttcgtcgagttctcccgccagcggggtctgtcgcccgacggacggtgcaaggcgttctcggactcggcggacggtgtcggctggtccgagggcgtgggcatgctggtcctggagcggcagtcggacgcgatccgcaacggccacgagatcctcgccgtgctgcgcggctccgccatcaaccaggacggcgcctccaacggcctcaccgcccccaacggtccctcgcagcagcgggtgatccgtcaggcgctggccagtggcggactgtcgacgggtgatgtggatgtcgtcgaggcgcacggtacgggtacgacgttgggtgacccgatcgaggcgcaggcgctgcttgcgacctacggccgggaccgtgacccggagcagccgctgctgctgggttcggtgaagtcgaacatcggtcacacgcaggccgcggcgggtgtcgcgggtgtgatcaagatggtcatgtcgatgcggcacggtgtgctcccgcgcaccctgcatgtcgatgccccgtcctcgcacgtcgactggaccgaaggggccgtggaactgctcaccgagcagacggagtggcccgagacggagcatgtgcgtcgtgcgggtgtctcctccttcggtatcagcggtaccaacgcccacgtcatcctggagcagccggcgaaggtgatccagggcacggtggtggctgcctcggcggcaccggagtcggacgtggtggagccctcggtggttccgtgggtgttgtccgccaagacgccggaggcgctgcgcgaccaggccgcccgcctgctcgccgcggtcaccgaggagccggcaccgcgtccggccgacgtggcccactccctgctcgtcacccgctcgctgttcgcccaccgcgcggtggtgctggcaggtgagcaccaggagacggtccgcgccctcaccgtactggcggcgggcgcgaccgacccgacggcggtgtcgggcacggtggcctccggtcgctcggcggcgttgttctcgggtcagggttcgcagcggctcggtatgggtcgtgagctgtacggccggttcccggtgttcgccgaggtcctggatgccgtgctcgctgttcttgacggggagttgggtggttcgctgcgggaggtgatgtggggtgaggatgccggcctgctgaacgagaccgggtggactcagcccgccctcttcgcggtcgaggtcgctctctatcggctggtggagtcgtggggtgtgaggccggactttgtggccggtcactccatcggtgagatcgctgccgcgcacattgccggggtgttctcgctggaggatgccgcccgcctcgtggccgctcgtggccgtctgatgcaggcgctcccggccggcggcgcgatggtcgccgtccaggccaccgaggacgaggtcgtcccgcatctgtccgacggggtgtcgatcgccgcgctcaacgggccgacgtccgtggtgctgtcgggcgttgaaagcgccgtactggagctggccgcgcggttcgaggcggagggtcgtaagaccacccggctccgcgtgtcccacgccttccactcgccgctgatggacccgatgctggacgagttccgggcggtggccgagggactctcctacgccgctccggtgatcccggtggtgtcgaacctgacgggttccctggccacggccgaggagctgtgctcgcccgagtactgggtccgtcacgtccgtgaggcggtccgcttcgccgacggggtggccgtccttgaggcgcagggcgtgaccacgttcctggagctgggccccgacggagttctctccgccatggcccaggagtcgctgaccggcgacgagaccgtcacgatcccgctgctgcgcaaggaccgcgacgaggagacgaccgccctgctggcccgcgccgggctgtacacacgggggatcgggggaggctggcaggacctgttggccggtaccggtgcccgccgggtgaacctgccgacgtacgccttccagcaccagtggttctggccgaccggccgggccgccgccggcggtgacgtccgtgccgccggtctcggctccgccgagcacccgctgctgggcgccgccgtcgaactcgccgcgggcgagggggtgctgttcaccggccgcctgtcgctgagctcgcacccgtggctggccgaccatgtcgtcatgggcaccgtcctgctgccgggcacggccctgctggagatggcggtccgcgcgggtgacgaggtcggctgcggccgggtcgaggaactcaccctcgccgcgccgctggtcctgcccgagcggggcgcgctccaggtgcaggtcgccgtcgccgccccggacgactccggccggcgcacggtcggggtgtactcgcgcctcgaaggcgccgacgagtcggtgtgggcccagcacgcggccggtgtcctggcgcccggcgccggcgccccggagacgggcctgggcaccgaggtgtggccgccgcgggacgccgagtcgctcgacaccgaggactgctaccaggcgttcgccgaggccggattcgcgtacggaccgctcttccaggggctgcgcgcggtgtggcgtcgcggtgaggaactcttcgccgaggtggcgctgcccgaggaaggcaccggtgaggcggccgagttcggtctgcaccccgcgctgctcgactccgcgctgcacgccaccatgctggccgccaccggcggcgaggagggcgacgggggaggtgccctgccgttctcgtgggagggcgtcgcgctgcacgcctcaggcgcctcggcgctgcgggtgcggctggcccccgccggcgacaacgcgctgtcggtgcacgcggtggacccggccggtgcgccggtggtcaccgtcggtcggctcctcacccgcacggtcaccggtgaccagctgaaggccgccggtgaggaccagaacacgctgttcggcgtcgactggacgccggtcgccgacacccgggccgcacggcccgcgtcggtcgcgatcgtcggagccgacccgttcggcctggccgacgcgttcgccggcgccggtctcacggccgtcacggcggccgacctcaccgcgctgacggcctcctccgaggccgccgtaccggacacggtgctcgtcccggtcgccggagccccggccgacggggcggacctgcccggcgcggtccgtgaggtcaccgggcgggtgctcgcactgctgcagcagtggcccgtcgaggaggcgctcgcggcctcgcggctggtgttcgtcacccgtggcgcggtcgccgtggacgacggcgggaccgcggacctcgcggcctccgcggtctggggcctggtgcggtcggcgcagacggagaaccccggctgcttcggcctgctcgacctcgacccggccgcggcggtcccgtccgcacggctcgtcgaggccctgacggccggcgccgaggaaccgcagctcgcgctgcgcggcatcgaggtcgtggccgcccgcctcgcccggtacgacgccgcacagccggagcccgcctccggcgccatggcggacggcacgtccggcacgttccggaccaccgggaccgtcctggtcaccggcggcaccggcggcctcggccggctcctcgcccggcacctggtgaccacgcacggcgtgcggagcctgctcctcgccagccgcaggggtctccaggcggacggcgcgggcaagctcgtcgccgaactggaggagatgggcgccgaggtcaccgtgcgggcgtgcgacgtcgccgacccggaggccgtggccgccctggtcgcggcggcaccgaagggccgtccgctgtcggcggtcgtccacacggccggtgtgctcgacgacggtgtggtcggctcgttgacgccggagcgcctcgacacggtgctgcggcccaaggcggacgcggtgtggcatctgcaccgggcgacccgcggcctggacctggacgcgttcgtcgtctactcgtccgtggccggtgtgttcggcggtgcggggcaggccaactacgcggcggccaacacgttcctggacgccctgatggcgcggcgccgcgccgacggactgcccggcgtgtccctcgcgtggggtccctgggagcaggacagcggcatgacgggcactctcacggacgccgaggccgaacggctggcgcgttccgggatgccgccgctgacggtcgagcagggccacgccctgttcgacgcggtggtcgcgggcgaacgtgcgctggcgctgccggtccggctggacctcgccgtactgcgtgccggtggtgaggtgcccgcgctgctgcgcggactgatccgggtgcccgcgcggcgggccgcggcccagagcgcggtgccgtccgcggacacgctcacccggcagctcgccggactgggcgcccaggagcaggaggaggcgctgctcaccctggtgcgcggacaggcggcgacggtgctcggccaccgggacggctcggcgatcggcagggagcgccagttccaggaactgggcttcgactcgctgaccgcggtcgagttccgcaaccggctcaacacggccaccggactgcggctgcccgccacgctgctgttcgactacccgacgccggccgacgtcgtccgctatctgcgcggacagctcgtcaccgacgatccgtcgggcgccggttcggtgctcgcggcgctcgaagggctggagaaggcgatcgccgcactcagcctcgacgacgcggaggagcaccggcgggtggccggccggatcgaggtcctcaggaccaagtgggccgcgctgggccgtacggacacggccgacgaggacgcctcgtcggacgacctggacggcgcgtccgacgaggacatgttcgcgctcctcgacgacgagctcggcctgagctgacccgttccgcgggccgccacggcggcccgcgggacccgccggggcccgggtgcccggctgaccgaaaacactggtggttttcccactcttctctctgtggaatggacatcgcgccccgacggccgtcggggcgcgatgtcatgtggctcacctggtgccgcgcgacaggcctcgttgccggccgcggcaccagtgagtcgcaccgcaagcgcagacggacaggtgaaggcatccggcgtgaccgctcccgcgggccgcgtcggccgaccgtccgcaggagcgcgccgtccggtccgtcccaaggagaacccccaggtgcgacacgaagccgctgcacccgagcggacggcccggggcgttcttcccggacgctccaccacgagccggccagccgggcggtctccgggcgatccgccgacgcggcagcctgccgtcacccgtctcccggacgacgatcgcgtccgccgcacgccggaacggatcc